(19) United States
(12) Patent Application Publication
(10) Pub. No.: US 2026/0041791 A1
(43) Pub. Date: Feb. 12, 2026

(54) COMPOSITIONS AND METHODS FOR TREATMENT OF ACHROMOTOPSIA

(71) Applicant: Adverum Biotechnologies, Inc., Redwood City, CA (US)

(72) Inventor: Brahim BELBELLAA, Redwood City, CA (US)

(73) Assignee: Adverum Biotechnologies, Inc., Redwood City, CA (US)

(21) Appl. No.: 18/879,984

(22) PCT Filed: Jul. 5, 2023
(86) PCT No.: PCT/US2023/069618

§ 371 Date: Dec. 30, 2024

Related U.S. Application Data
(60) Provisional application No. 63/367,790, filed on Jul. 6, 2022.
(60) Provisional application No. 63/367,723, filed on Jul. 6, 2022.
(60) Provisional application No. 63/397,528, filed on Aug. 12, 2022.

(51) Int. Cl. A61K 48/00; A61K 38/17; C12N 15/86

(52) U.S. Cl. CPC A61K 48/0058; A61K 38/177; A61K 48/0075; A61K 48/0083; C12N 15/86; C12N 2750/14143; C12N 2800/22

(57) ABSTRACT

Provided is the intravitreal or subretinal dosing of recombinant adeno-associated virus (rAAV)-based gene therapies for the treatment of color vision deficiencies such as achromotopsia.

### CROSS-REFERENCE TO RELATED APPLICATIONS

This application is a 371 National Stage filing and claims the benefit under 35 U.S.C. § 120 of International Application No. PCT/US2023/069618, filed Jul. 5, 2023, which claims priority under 35 U.S.C. § 119 (e) to U.S. Provisional Application No. 63/367,723 filed Jul. 6, 2022, U.S. Provisional Application No. 63/367,790 filed Jul. 6, 2022, and U.S. Provisional Application No. 63/397,528 filed Aug. 12, 2022; each of which is incorporated by reference in its entirety.

### STATEMENT REGARDING THE SEQUENCE LISTING

The official copy of the Sequence Listing is submitted concurrently with the specification as an WIPO Standard ST.26 formatted XML file with file name “17234-039WO1_SequenceListing.xml”, a creation date of Jun. 28, 2023, and a size of 72 kilobytes. This Sequence Listing filed via USPTO Patent Center is part of the specification and is incorporated in its entirety by reference herein.

### TECHNICAL FIELD

The present disclosure relates to methods of treating achromotopsia in an individual that comprise administering a single unit dose of a recombinant adeno-associated virus (rAAV) particles encoding a CNGB3 gene to an eye of an individual.

### INCORPORATION BY REFERENCE

All references cited herein, including patent applications and publications, are incorporated by reference in their entirety.

### BACKGROUND

Achromotopsia is a rare autosomal recessive disease that results in retinal degeneration affecting all three types of cone photoreceptor cells that results in reduced visual acuity, photophobia, hemeralopia, and severe loss of color discrimination. Mutation in the CNGB3 genes accounts for greater than 90% of patient and results in complete Achromatopsia, meaning that they have significant impairment in color discrimination and central visual acuity. Currently, there is no approved therapeutic treatment for achromotopsia. Therefore, there is a need in the art for therapies for achromotopsia.

### BRIEF SUMMARY OF THE INVENTION

In some aspects, the invention provides methods for treating achromotopsia disease in an individual, the method comprising administering of recombinant adeno-associated virus (rAAV) particles to one eye of the individual, wherein the individual is a human, wherein the rAAV particles comprise: a) a nucleic acid encoding a CNGB3 polynucleotide and flanked by AAV2 inverted terminal repeats (ITRs), and b) an AAV2 capsid protein comprising an amino acid sequence LGETTRP (SEQ ID NO:8) inserted between positions 587 and 588 of the capsid protein, wherein the amino acid residue numbering corresponds to an AAV2 VP1 capsid protein.

In some embodiments of the invention, the unit dose of rAAV particles is about 2×10¹⁰ vector genomes (vg) or less. In some embodiments, the unit dose of rAAV particles is between about 6×10⁹ to about 6×10¹¹ vector genomes per eye (vg/eye). In some embodiments, the unit dose of rAAV particles is between about 2×10¹⁰ to about 2×10¹¹ vector genomes per eye (vg/eye). In some embodiments, the unit dose of rAAV particles is about 2×10¹⁰ or about 3×10¹⁰ vector genomes per eye (vg/eye). In some embodiments, the unit dose of rAAV particles is about 4×10¹⁰ vector genomes per eye (vg/eye). In some embodiments, the unit dose of rAAV particles is about 1×10¹⁰ vector genomes per eye (vg/eye). In some embodiments, the unit dose of rAAV particles is about 1×10¹¹ vector genomes per eye (vg/eye). In some embodiments, the unit dose of rAAV particles is about 6×10¹⁰ vector genomes per eye (vg/eye). In some embodiments, the individual has one or more symptoms of achromotopsia. In some embodiments, the invention further comprises administering a unit dose of rAAV particles to both eyes of the individual. Administering a unit dose of rAAV particles to the contralateral eye of the individual may be done either simultaneously with a first eye or subsequent to treatment of the first eye.

In some embodiments, the rAAV particles in the pharmaceutical formulation are present at a concentration of about 1×10¹⁰ vg/ml to about 1×10¹³ vg/ml. In some embodiments, the rAAV particles in the pharmaceutical formulation are present at a concentration of about 1×10⁹ vg/ml to about 6×10¹⁴ vg/ml. In certain embodiments, the rAAV particles in the pharmaceutical formulation are present at a concentration of about 1×10⁹ vg/ml to about 2×10⁹ vg/ml, about 2×10⁹ vg/ml to about 3×10⁹, about 3×10⁹ vg/ml to about 4×10⁹, about 4×10⁹ vg/ml to about 5×10⁹, about 5×10⁹ vg/ml to about 6×10⁹, about 6×10⁹ vg/ml to about 7×10⁹, about 7×10⁹ vg/ml to about 8×10⁹, about 8×10⁹ vg/ml to about 9×10⁹, about 9×10⁹ vg/ml to about 10×10⁹, about 10×10⁹ vg/ml to about 1×10¹⁰, about 1×10¹⁰ vg/ml to about 2×10¹⁰, about 2×10¹⁰ vg/ml to about 3×10¹⁰, about 3×10¹⁰ vg/ml to about 4×10¹⁰, about 4×10¹⁰ vg/ml to about 5×10¹⁰, about 5×10¹⁰ vg/ml to about 6×10¹⁰, about 6×10¹⁰ vg/ml to about 7×10¹⁰, about 7×10¹⁰ vg/ml to about 8×10¹⁰, about 8×10¹⁰ vg/ml to about 9×10¹⁰, about 9×10¹⁰ vg/ml to about 10×10¹⁰, about 10×10¹⁰ vg/ml to about 1×10¹¹, about 1×10¹¹ vg/ml to about 2×10¹¹, about 2×10¹¹ vg/ml to about 3×10¹¹, about 3×10¹¹ vg/ml to about 4×10¹¹, about 4×10¹¹ vg/ml to about 5×10¹¹, about 5×10¹¹ vg/ml to about 6×10¹¹, about 6×10¹¹ vg/ml to about 7×10¹¹, about 7×10¹¹ vg/ml to about 8×10¹¹, about 8×10¹¹ vg/ml to about 9×10¹¹, about 9×10¹¹ vg/ml to about 10×10¹¹, about 1×10¹² vg/ml to about 2×10¹², about 2×10¹² vg/ml to about 3×10¹², about 3×10¹² vg/ml to about 4×10¹², about 4×10¹² vg/ml to about 5×10¹², about 5×10¹² vg/ml to about 6×10¹², about 6×10¹² vg/ml to about 7×10¹², about 7×10¹² vg/ml to about 8×10¹², about 8×10¹² vg/ml to about 9×10¹², about 9×10¹² vg/ml to about 10×10¹², about 1×10¹³ vg/ml to about 2×10¹³, about 2×10¹³ vg/ml to about 3×10¹³, about 3×10¹³ vg/ml to about 4×10¹³, about 4×10¹³ vg/ml to about 5×10¹³, about 5×10¹³ vg/ml to about 6×10¹³, about 6×10¹³ vg/ml to about 7×10¹³, about 7×10¹³ vg/ml to about 8×10¹³, about 8×10¹³ vg/ml to about 9×10¹³, about 9×10¹³ vg/ml to about 10×10¹³, about 1×10¹⁴ vg/ml to about 2×10¹⁴, about 2×10¹⁴ vg/ml to about 3×10¹⁴, about 3×10¹⁴ vg/ml to about 4×10¹⁴, about 4×10¹⁴ vg/ml to about 5×10¹⁴, or about 5×10¹⁴ vg/ml to about 6×10¹⁴ vg/mL. In some embodiments, the pharmaceutical formulation comprises about 6×10¹¹ vg/mL to about 6×10¹² vg/mL of rAAV particles. In some embodiments, the pharmaceutical formulation comprises about 6×10¹² vg/mL of rAAV particles. In some embodiments, the pharmaceutical formulation comprises about 6×10¹¹ vg/mL of rAAV particles.

In some embodiments of the invention, the nucleic acid comprises the nucleic acid sequence encoding the human CNGB3 gene (SEQ ID NO: 10) or a sequence having at least 75%, at least 80%, at least 85%, at least 90%, at least 95%, or at least 97% identity thereto.

In some embodiments of the invention, the nucleic acid comprises a nucleic acid sequence encoding a human CNGB3 protein (SEQ ID NO: 11) or a sequence having at least 75%, at least 80%, at least 85%, at least 90%, at least 95%, or at least 97% identity thereto.

In some embodiments of the invention, the nucleic acid comprises a nucleic acid sequence encoding a human CNGB3 protein as set forth in SEQ ID NO: 20 or a sequence having at least 75%, at least 80%, at least 85%, at least 90%, at least 95%, or at least 97% identity thereto.

In some embodiments of the invention, the AAV2 capsid protein comprises the amino acid sequence LALGETTRPA (SEQ ID NO:6) inserted between positions 587 and 588 of the capsid protein, wherein the amino acid residue numbering corresponds to an AAV2 VP1 capsid protein. In some embodiments, the AAV2 capsid protein comprises the amino acid sequence LGETTRP (SEQ ID NO:8) or ISDQTKA (SEQ ID NO:29) inserted between positions 587 and 588 of the AAV2 VP1 comprising the sequence of SEQ ID NO: 7. In some embodiments, the AAV2 capsid protein comprises the amino acid sequence LALGETTRPA (SEQ ID NO:6) inserted between positions 587 and 588 of the AAV2 VP1 comprising the sequence of SEQ ID NO: 7. In some embodiments, the rAAV particles comprise an AAV2 VP1 capsid protein comprising a GH loop that comprises the amino acid sequence of SEQ ID NO: 9 or an amino acid sequence having at least 90% sequence identity to SEQ ID NO: 9.

In some aspects, the invention provides a unit dose of recombinant adeno-associated virus (rAAV) particles for use in a method for treating achromotopsia in an individual, the method comprising administering said unit dose to one or both eye(s) of the individual, wherein the individual is a human, wherein the rAAV particles comprise a) a nucleic acid encoding the CNGB3 gene and flanked by a 145 nt partial AAV2 inverted terminal repeats (ITRs), and b) an AAV2 capsid protein comprising an amino acid sequence LGETTRP (SEQ ID NO:8) or ISDOTKA (SEQ ID NO:29) inserted between positions 587 and 588 of the capsid protein, wherein the amino acid residue numbering corresponds to an AAV2 VP1 capsid protein.

Other objects, features and advantages of the present invention will become apparent from the following detailed description. It should be understood, however, that the detailed description and specific examples, while indicating preferred embodiments of the invention, are given by way of illustration only, since various changes and modifications within the scope and spirit of the invention will become apparent to one skilled in the art from this detailed description.

### BRIEF DESCRIPTION OF THE DRAWINGS

FIGS. 1A-1H illustrate the vector design and successive sequence optimization to increase manufacturing yield, expression level and safety. (FIG. 1A) Initial achromatopsia vector “pADV697”, encoding the human CNGB3 cDNA wild type sequence and the MNTC regulatory elements including the LCR enhancer, the OPN1LW1 core promoter, the chimeric intron, 5′UTR and the SV40 pA as annotated in the schema (figures) and the sequence listing. Total payload size is 5,174 nt, including ITR. (FIG. 1B) Achromatopsia vector “pADV781”, encoding part of the MNTC regulatory elements (LCR enhancer, OPN1LW1 core promoter, chimeric intron, 5′UTR), the human CNGB3 cDNA codon optimized sequence and the short synthetic polyadenylation sequence. Residual non-coding DNA sequence(s) was/were deleted from the vector payload. Total payload size is 4,753 nt, including ITR. (FIG. 1C) Achromatopsia vector “pADV963”, encoding part of the MNTC regulatory elements (LCR enhancer, core promoter and chimeric intron), the partial human CTNNB1 5′UTR sequence, the human CNGB3 cDNA codon optimized sequence and the short synthetic polyadenylation sequence. Total payload size is 4,830 nt, including ITR. (FIG. 1D) Alternative achromatopsia vector “pADV854”, encoding the PR1.7 promoter, the human CNGB3 cDNA codon optimized V11 sequence and the SV40 polyadenylation sequence. Total payload size is 4,725 nt, including ITR. (FIG. 1E) Control vector “pADV843” encoding the optimized eGFP cDNA sequence and MNTC regulatory elements (LCR enhancer, core promoter, chimeric intron, 5′UTR, optimized Kozak and SV40 pA). Total payload size is 3,237 nt, including ITR. (FIG. 1F) Achromatopsia vector “pADV960”, encoding part of the MNTC regulatory elements (LCR enhancer, core promoter and chimeric intron), an optimized 10 nt optimized lead sequence upstream of the optimized Kozak sequence, the human CNGB3 cDNA codon optimized sequence and the short synthetic polyadenylation sequence. Total payload size is 4,741 nt, including ITR. (FIG. 1G) Achromatopsia vector “pADV961”, encoding part of the MNTC regulatory elements (LCR enhancer, core promoter and chimeric intron), the human CNGB3 cDNA codon optimized sequence and the short synthetic polyadenylation sequence. Total payload size is 4,730 nt, including ITR. (FIG. 1H) Achromatopsia vector “pADV962”, encoding part of the MNTC regulatory elements (LCR enhancer, core promoter and chimeric intron), the partial human TUBB 5′UTR sequence, the human CNGB3 cDNA codon optimized sequence and the short synthetic polyadenylation sequence. Total payload size is 4,741 nt, including ITR. Abbreviations: ITR, inverse terminal repeat from AAV2; LCR, human opsin locus control region; OPN1LW promoter, human opsin 1, long wave sensitive gene core promoter; 5′UTR, mRNA 5′ untranslated region; OK, optimized Kozak sequence; hCNGB3, cDNA encoding the human cyclic nucleotide gated channel subunit beta 3; SV40 pA, Simian virus 40 polyadenylation sequence; SPA, short synthetic polyadenylation sequence; CTNNB1 5′UTR, 5′UTR from the human catenin beta 1 gene; CpG, CG or GC dinucleotide; LS, optimized lead sequence; TUBB, human beta tubulin gene.

FIGS. 2A-B illustrate a Western blot analysis of CNGB3 expression level, following transfection of HEK293 cells with transgene plasmid vectors encoding incremental optimization of the expression cassette. HEK293 cells were transfected in P6-multiwell with six different plasmid vectors, in duplicate. These include pADV697, pADV781 and pADV963 described in FIG. 1A-C. Cells were harvested 48 hours later and total protein was extracted. SDS PAGE analysis was performed by loading 7 mg of protein sample, on 4-12% gradient Tris-glycine polyacrylamide gel, followed by transfer onto PVDF membrane using the iBlot2. The PVDF membranes were immunoblotted a first time against CNGB3 antibodies (rabbit anti-CNGB3 antibodies; ThermoFisher Scientific, catalog no. PA566068), then with secondary HRP-conjugated antibody (goat anti-rabbit antibodies; Cell Signaling, catalog no. 7074) and finally the signal was imaged. For signal normalization, the membrane was immunoblotted a second time against GAPDH. (FIG. 2A) Representative immunoblot image showing CNGB3 and GAPDH levels, from duplicate transfection with six different plasmids vector. Lanes 1a-b correspond to duplicate transfection with pADV697, 2a-b to pADV781, 5a-b to pADV960, 6a-b to pADV961, 7a-b to pADV962 and 8a-b to pADV963. (FIG. 2B) Normalization of CNGB3 signal to GAPDH and average for duplicate transfection experiments. Abbreviations: MW, molecular weight; CNGB3, cyclic nucleotide gated channel subunit beta 3 protein; GAPDH, glyceraldehyde-3-phosphate dehydrogenase; MNTC, composite promoter encoding LCR, cone opsin core promoter and chimeric intron; 5′UTR, mRNA 5′ untranslated region; 5UTR.22, synthetic 5′UTR sequence; 5UTR. 1, synthetic 5′UTR sequence; 5UTR.2, minimal synthetic 5′UTR sequence; 5UTR.hTUBB, human beta tubulin 5′UTR sequence; 5UTR.hCTNNB1, human CTNNB1 5′UTR sequence; OK, optimized Kozak sequence; CNGB3, cDNA encoding the human cyclic nucleotide gated channel subunit beta 3; SV40 pA, Simian virus 40 polyadenylation sequence; SPA, short synthetic polyadenylation sequence; CNGB3 wt, human CNGB3 cDNA wild type sequence; CNGB3CoOpt11, human CNGB3 cDNA codon optimized sequence.

FIGS. 3A-3D illustrate an experimental design of mouse study and longitudinal function read-out to evaluate of functional rescue following test article injection in achromatopsia (ACHM) mouse model. The 6 mouse groups composing this study were gender-balanced and include either wild-type (WT) or knock-out (KO) for both allele of cyclic nucleotide gated channel subunit beta 3 gene (Cngb3). Both WT and KO mice have identical C57/B6J genetic background. In brief, WT mice were not injected with any test article (group #1; n=10) or treated with formulation buffer (group #2; n=8). Cngb3 KO mice were not injected with test article (group #3; n=16) or treated with either formulation buffer (group #4; n=5), AAV2.7m8-pADV843-MNTC-eGFP-SV40 pA (group #5; n=10), AAV2.7m8-pADV963-MNTC-5UTR.hCTNNB1-OK-CNGB3coV11-SPA (group #6; n=7) or AAV2yF-pADV854-PR1.7-CNGB3coV11-SV40 pA (group #7; n=8). The timeline chart represents the mice age in weeks, at which the treatment and functional read-out were performed. Functional read-out to assess retina function and health in-vivo were performed at prior to injection at baseline (approximately 5 weeks of age), then at 8- and 16-weeks post-injection, as described in the schematic. At approximately 6 weeks of age, treated mice received a single 1 μL volume subretinal injection, in left and right eyes, with approximately 1×10¹⁰ vg/eye. Between 16-18 weeks after dosing (i.e., approximately 22-24 weeks of age), mice were sacrificed, and the left eye was processed into retina whole-mount, which was then labelled for CNGB3 by immunohistochemistry. Mice body weight were measured weekly. Abbreviations: CNGB3, cyclic nucleotide gated channel subunit beta 3 protein; MNTC, composite promoter encoding LCR, cone opsin core promoter and chimeric intron; PR1.7, recombinant promoter derived from the M/L cone opsin genes; 5′UTR, mRNA 5′ untranslated region; 5UTR.hCTNNB1, human CTNNB1 5′UTR sequence; OK, optimized Kozak sequence; SV40 pA, Simian virus 40 polyadenylation sequence; SPA, short synthetic polyadenylation sequence; CNGB3CoOpt11, human CNGB3 cDNA codon optimized sequence.

FIGS. 4A-4E illustrate that subretinal injection of AAV2.7m8-pADV843-MNTC-eGFP in 1.5-month-old Cngb3 Knock-Out (KO) mice, leads to robust GPF expression specifically in cone photoreceptor, widespread across the retina and sustained over 16 weeks post-injection. In order to assess the efficient dose and route of administration, in addition to confirm the profile of transduction in Cngb3 KO mice, the vector AAV2.7m8-pADV843-MNTC-eGFP-SV40 pA was constructed as described in FIG. 1E. At approximately 6-weeks of age, n=5 female and n=5 male KO mice were injected with 1 μL, i.e., approximately 1×10¹⁰ vector genome. Ocular fundi were performed at 8- and 16-weeks post-injection to assess the kinetics of GFP expression in vivo (FIG. 4A). At study termination (approximately 22-24 weeks of age), all animals were sacrificed, and their left eyes and right eyes were processed respectively into retina wholemount (FIG. 4B-FIG. 4C), FFPE whole eye cross sections (FIG. 4D) or frozen retina cross section (FIG. 4E), to assess GFP expression level and distribution. (FIG. 4B) Macroscope imaging of the native GFP signal from retina wholemount, observed at study termination. (FIG. 4C) Quantification of the retina surface positive for GFP signal. (FIG. 4D) Slide scanner imaging of whole eye cross section, following fluorescent immunolabelling of eGFP. (FIG. 4E) Microscope imaging of retina cross sections, following fluorescent immunolabelling of eGFP.

FIGS. 5A-5D shows the electroretinogram measurement of cones and rod photoreceptor's function, before treatment with test article. At approximately 6 weeks of age, wild-type (WT) and Cngb3 knock-out (KO), retina baseline function was evaluated by scotopic (A-B) and photopic (C-D) electroretinogram (ERG). Briefly, after overnight dark adaptation, animals were anesthetized by intraperitoneal injection of 85 mg/kg ketamine and 14 mg/kg xylazine. ERGs were recorded in a ColorDome using an Espion V6 Software (Diagnosys LLC, Lowell, MA). For assessment of scotopic responses, a stimulus intensity of 77 cd s m-2 was presented to dark-adapted dilated mouse eyes. To evaluate photopic responses, mice were adapted to a 25 cd s m-2 light for 5 minutes, then a light intensity of 77 cd s m-2 was administered. Data for each one of the seven experimental groups are represented as mean with SD, with each dot corresponding to an individual mouse eye. One-way ANOVA with FDR 5% statistical test: ns, non-significant; *q<0.05; **q<0.01; ***q<0.001; ****q<0.0001.

FIGS. 6A-6D show electroretinogram measurements of cones and rod photoreceptor's function, 8-weeks after treatment. At approximately 15 weeks of age, photopic (FIGS. 6A-6B) and scotopic (FIGS. 6C-6D) electroretinogram (ERG) were measured in all mice, wild-type (WT) and Cngb3 knock-out (KO), treated or not with a test article. Briefly, after overnight dark adaptation, animals were anesthetized by intraperitoneal injection of 85 mg/kg ketamine and 14 mg/kg xylazine. ERGs were recorded in a ColorDome using an Espion V6 Software (Diagnosys LLC, Lowell, MA). For assessment of scotopic responses, a stimulus intensity of 77 cd s m-2 was presented to dark-adapted dilated mouse eyes. To evaluate photopic responses, mice were adapted to a 25 cd s m-2 light for 5 minutes, then a light intensity of 77 cd s m-2 was administered. Data are represented as mean with SD, with each dot corresponding to an individual mouse eye. One-way ANOVA with FDR 5% statistical test: ns, non-significant; *q<0.05; **q<0.01; q<0.001; ****q<0.0001.

FIGS. 7A-7B show electroretinogram measurements of rod photoreceptor's function, 16-weeks after treatment. At approximately 22-23 weeks of age, scotopic ERG(s) were recorded in all mice, wild-type (WT) and Cngb3 knock-out (KO), treated or not with a test article. Like at prior timepoints, scotopic responses were measured in dark-adapted dilated mouse eyes, with single-flash light stimulation at 77 cd s m-2 intensity. (FIG. 7A) Scotopic a-wave amplitude reported in μV. (FIG. 7B) Scotopic b-wave amplitude reported in μV. Data are represented as mean with SEM, with each dot corresponding to an individual mouse eye.

FIGS. 8A-8C show electroretinogram measurements of cone photoreceptor's function, 16-weeks after treatment. At approximately 22-23 weeks of age, serial-flash photopic ERG(s) were recorded in all mice, wild-type (WT) and Cngb3 knock-out (KO), treated or not with a test article. Like at prior timepoints, photopic responses were measured in mice light-adapted for 5 minutes at 25 cd s m-2. Briefly, photopic flash recordings measure consists in the average of 25 responses for each intensity with a 60 second light adaptation interval between each step. Light intensities used were 0.1, 1, 3, 5, 10 and 20 cd s m-2. Responses were differentially amplified, averaged, and stored according to the Espion V6 Software. (FIG. 8A-FIG. 8B) Photopic b-wave amplitude is reported in μV, following (FIG. 8B) light stimulation at 10 cd s m-2 (FIG. 8A) and at 20 cd s m-2 (FIG. 8B). One-way ANOVA with FDR 5% statistical test: ns, non-significant; *q<0.05; **q<0.01; ***q<0.001; ****q<0.0001. (FIG. 8C) Cone photoreceptor dose response curve shown with increasing b-wave amplitude as function of the increasing light intensities in the serial recording. To assess intergroup difference at each given timepoint separately, one-way ANOVA with FDR 5% statistical test was performed (ns, non-significant; #q<0.05; ##q<0.01; ###q<0.001; ###q<0.0001). To assess longitudinal intergroup difference, Two-way ANOVA mixed model analysis for repeat measures was performed with FDR (ns, non-significant; *q<0.05; **q<0.01; ***q<0.001; ***q<0.0001). Data are represented as mean with SD, with each dot corresponding to an individual mouse eye.

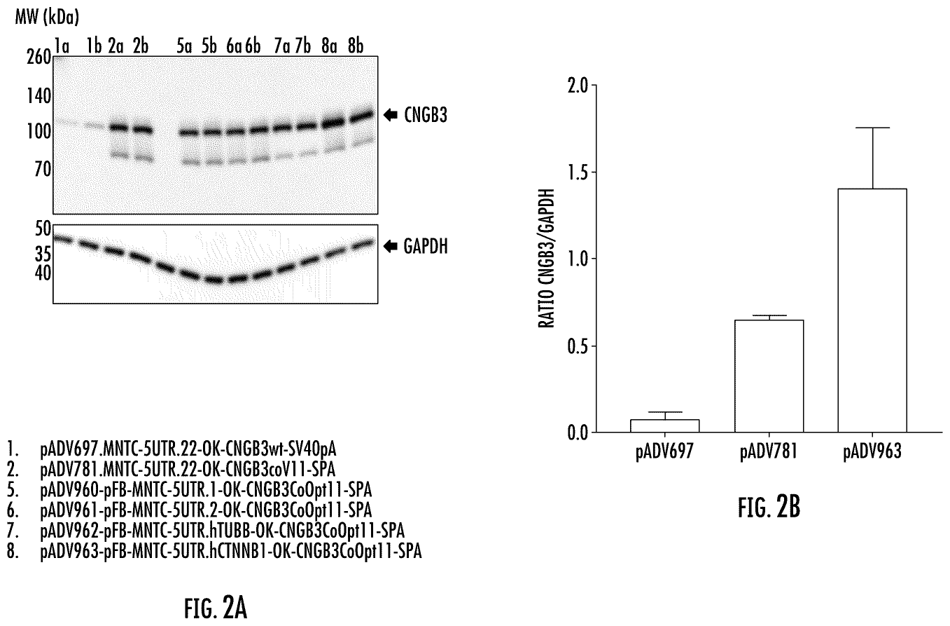

FIG. 2 shows representative photopic electroretinogram (ERG) traces from naïve and treated wild-type and Cngb3 Knock-Out mice, recorded at baseline and after treatment. Photopic ERGs recording were performed at baseline (1.5-month of age; 1.5M), at 8-weeks post-injection (3.5-months of age; 3.5M) and 16-weeks post-injection (5.5-months of age; 5.5M). Abbreviations: WT, wild-type mice naïve; Cngb3−/−, Cngb3 KO mice naïve; Cngb3−/− form, Cngb3 KO mice treated with formulation buffer; Cngb3−/− 2.7m8, Cngb3 KO mice treated with AAV2.7m8-pADV963-MNTC-CNGB3-SPA; Cngb3−/− 2.tYF, Cngb3 KO mice treated with AAV2tYF.pADV854-PR1.7-CNGB3-SV40 pA.

FIGS. 10A-10B show the evaluation of visual acuity rescue at 16-weeks post-treatment, by optokinetic test (OKT). (FIG. 10A) OKT in Cngb3 was performed on three successive dates, on wild-type mice naïve (n=8); Cngb3 KO mice naïve (n=7), Cngb3 KO mice treated with formulation buffer (n=5), Cngb3 KO mice treated with AAV2.7m8-pADV963-MNTC-CNGB3-SPA (n=7); Cngb3 KO mice treated with AAV2tYF-pADV854-PR1.7-CNGB3-SV40 (n=8). Data are represented as mean with SD. (FIG. 10B) OKT score average from 3 consecutive measurements. Data are represented as mean with SD. One-way ANOVA with FDR 5% statistical test: ns, non-significant; *q<0.05; **q<0.01; ***q<0.001; ****q<0.0001. OKT was performed using the Optomotry system (Cerebral Mechanics Inc.), where mice are placed onto a platform surrounded by 4 LCD screens which resides within a light-protected box. Visual stimuli consist of vertical lines rotating at varying frequencies and are presented to the mouse via the LCD screens. The operator visualizes and scores optokinetic tracking reflexes from a digital camcorder which is mounted on the top of the OKT system.

FIG. 11 shows the representative microscope images of whole mount retinas from wild-type and Cngb3 knock-out (KO) mice, treated with test article or naïve, sacrificed at between 16-18 weeks post-injection and labelled for human CNGB3 protein expression. At study endpoint (21-24 weeks of age), WT and Cngb3 KO mice were sacrificed, and their eyes processed into retina wholemount. Following immunostaining with an antibody recognizing both human and mouse CNGB3 protein, the fluorescent labelling was imaged with identical magnification and exposure time, in all experimental conditions. The punctate labelling observed in WT mice and restored in Cngb3 KO mice treated with AAV-CNGB3, corresponds to the typical pattern of cone photoreceptor distribution in the mouse retina. CNGB3 expression was widespread in the retina of WT mice. However, in Cngb3 KO mice, the signal was localized to the bleb where AAV-CNGB3 was subretinal injected.

FIG. 12 shows representative microscope imaging of paraffin retinas cross sections from wild-type and Cngb3 knock-out (KO) mice, treated with test article or naïve, sacrificed at 16-18 weeks post-injection and labelled for CNGB3 protein expression. At study endpoint (21-24 weeks of age), WT and Cngb3 KO mice were sacrificed, and their eyes fixed in formaldehyde and embedded in paraffin, prior to sectioning. Tissue sections were labeled with DAPI and with a rabbit polyclonal antibody recognizing specifically the human CNGB3 protein and not the mouse endogenous CNGB3 protein. Tissue sections were imaged with a slide scanner, using the identical acquisition parameters, and then displayed at low and high (40×) magnification.

FIGS. 13A-13H illustrate the Western blot analysis of CNGB3 expression level, following transfection of HEK293 cells with expression plasmid vectors encoding for the human Cngb3 cDNA sequence, either wild type or codon optimized. In addition to the native cDNA sequence (CNGB3 wt), six codon optimized cDNA sequences were generated in silico, with various codon bias and CpG number i.e., CNGB3v5, CNGB3v10, CNGB3v11, CNGB3v12, CNGB3v15 and CNGB3coGS. These cDNA sequences were subcloned in the same plasmid backbone, where gene expression is regulated by the CMV promoter and the SV40 pA. Three different cell lines (HEK293, HELA and Huh7) were transfected in P6-multiwell without DNA or with 0.6 μg of DNA frp, one of these expression plasmids, all diluted at 150 ng/μL. Cells were harvested after 48 hours, and total protein was extracted. SDS PAGE analysis was performed by loading 10 μg of protein sample, on 4-12% gradient Tris-glycine polyacrylamide gel, followed by transfer onto PVDF membrane using the iBlot2. The PVDF membranes were immunoblotted a first time against CNGB3 antibodies, then with secondary HRP-conjugated antibody. For signal normalization, the membrane was immunoblotted a second time against GAPDH. Lanes 1(a-b) correspond to transfection with no DNA, 2(a-b) to GFP expressing control plasmid, 3(a-b) to CNGB3 wt, 4(a-b) to CNGB3v5, 5(a-b) to CNGB3v10, 6(a-b) to CNGB3v11, 7(a-b) to CNGB3v12, 8(a-b) to CNGB3v15, 9(a-b) to CNGB3coGS. (A-B) HEK293 transfected in duplicate. Biological replicates are annotated as a and b on the western blot image (FIG. 13A). CNGB3 protein level normalized to GAPDH level. Biological replicates are reported as datapoint (FIG. 13B). (FIG. 13C-FIG. 13D) Western blot analysis of transfected HELA (C). CNGB3 protein level normalized to GAPDH level (FIG. 13D). (FIG. 13E-FIG. 13F) Western blot analysis of transfected Huh7 (E). CNGB3 protein level normalized to GAPDH level (FIG. 13F). (FIG. 13G) Graphical representation of CNGB3 expression level across the three cell lines evaluated. (H) Graphical comparative representation of codon bias, CpG and sequence similarity across the different CNGB3 encoding cDNA sequences.

FIGS. 14A-14H illustrate the Western blot analysis of CNGB3 expression level, following transfection of HEK293 cells with expression plasmid vectors encoding for the human Cngb3 cDNA sequence, with various combinations of 5′untranslated region (UTR) and intron sequences.

As described in Table 1, nineteen vector constructs were generated with various 5′UTR and intron sequence, but all encoding the same human CNGB3 codon optimized cDNA. All of these sequence combinations were subcloned in the same parental expression plasmid construct and derived directly from pADV781. These constructs were co-transfected in duplicated in HEK293 cells (seeded in P6-multiwell; 1 μg DNA per well), with the CRISPRa SAM system to transactivate the MNTC promoter, as described in the DETAILED DESCRIPTION section. Cells were harvested after 48 hours, and total protein was extracted. SDS PAGE analysis was performed by loading 15 μg of protein sample, on 4-12% gradient Tris-glycine polyacrylamide gel, followed by transfer onto PVDF membrane using the iBlot2. The PVDF membranes were immunoblotted a first time against CNGB3 antibodies, then with secondary HRP-conjugated antibody. For signal normalization, the membrane was immunoblotted a second time against GAPDH. (A-F) Western blot analysis of CNGB3 protein level, following transfection with pADV697 (lanes 1a-b), pADV781 (lanes 2a-b), pADV958 (lanes 3a-b), pADV959 (lanes 4a-b), pADV960 (lanes 5a-b), pADV961 (lanes 6a-b), pADV962 (lanes 7a-b), pADV963 (lanes 8a-b), pADV964 (lanes 9a-b), pADV965 (lanes 10a-b), pADV966 (lanes 11a-b) or pADV967 (lanes 12a-b). (B, D, F) CNGB3 protein level normalized to GAPDH level and pADV697 encoding the wild type cDNA sequence encoding CNGB3. (G) Western blot analysis of CNGB3 protein level, following transfection with the GFP control vector pADV843 (lane 1) or the CNGB3 encoding plasmid vector pADV697 (lanes 2a-b), pADV781 (lanes 3a-b), pADV963 (lanes 4a-b), pADV1001 (lanes 5a-b), pADV1002 (lanes 6a-b), pADV1003 (lanes 7a-b), pADV1004 (lanes 8a-b), pADV1005 (lanes 9a-b), pADV1006 (lanes 10a-b), pADV1007 (lanes 11a-b) or pADV1008 (lanes 12a-b). (H) CNGB3 protein level normalized to GAPDH level and pADV697 encoding the wild type cDNA sequence encoding CNGB3.

FIGS. 15A-15B show the microscopy imaging of HEK293 and HELA cells, following transfection with six different expression plasmid vectors all encoding the CMV promoter and eGFP reporter gene, but with different polyadenylation signal sequence. Five expression plasmids with different polyA sequence were derived from the initial expression plasmid pAVA005, which encodes the CMV promoter, chimeric intron, optimized Kozak, the codon-optimized eGFP cDNA and SV40 polyadenylation sequence (SV40 pA). The expression plasmid pADV782 encodes the 49 nucleotide-long (nt) synthetic short polyA (SPA), pADV907 the 49 nt SPA with a downstream 40 nt spacer, pADV893 the 224 nt bovine growth hormone polyA (bGHpA), pADV899 the 486 nt human growth hormone polyA (hGHpA), pADV904 the 550 nt rabbit beta globin polyA (RBGpA). Two days post-transfection the cells nuclei were labelled with Hoechst and the fluorescent signals was acquired using an inverse confocal microscope. The GFP and Hoechst signal were imaged at constant time exposure, respectively for HEK293 and for HELA cells. Here, pADV782 encoding for the SPA demonstrated very robust GFP expression and signal, similar to other candidates polyA, despite being the shorter sequence.

FIGS. 16A-16C show the the CRISPRa SAM system and the in vitro screening of guide RNA, used to induce the transactivation of the cone photoreceptor specific promoters MNTC and PR1.7. (FIG. 16A) As described in the Material and Method section, this CRISPRa SAM system relies on the transfection of the two plasmids pCas-Guide-CRISPRa and pCas-CRISPR-Enhancer, in addition to the target plasmid encoding for the GFP reporter gene. Seven guide RNA (gRNA) were designed to target the MNTC and/or the PR1.7 promoter and subcloned in the pCas-Guide-CRISPRa plasmid. An additional scramble gRNA was used as control. Each one of these eight gRNA was evaluated in duplicate, by triple transfection of HEK293 seeded in P6-well plate. Two days later, the transfected cells were imaged in bright light and GFP fluorescent light, with constant exposure time and excitation-light intensity. (FIG. 16B) Representative images of HEK293 cells, following transfection with the pADV843-MNTC-eGFP-SV40 pA expression plasmid, the pCas-CRISPR-Enhancer plasmid and one of the eight pCas-Guide-CRIPSRa plasmids. Additionally, were included here the negative non-transfected control and positive control of transfection with pAVA005-CMV-eGFP-SV40 pA plasmid. (FIG. 16C) Representative images of HEK293 cells, following transfection with the pADV853-PR1.7-eGFP-SV40 pA expression plasmid, the pCas-CRISPR-Enhancer plasmid and one of the eight pCas-Guide-CRIPSRa plasmids.

### DETAILED DESCRIPTION

The invention will now be described in detail by way of reference only using the following definitions and examples. All patents and publications, including all sequences disclosed within such patents and publications, referred to herein are expressly incorporated by reference.

Numeric ranges are inclusive of the numbers defining the range. The term about is used herein to mean plus or minus ten percent (10%) of a value. For example, “about 100” refers to any number between 90 and 110.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention which can be had by reference to the specification as a whole. Accordingly, the terms defined immediately below are more fully defined by reference to the specification as a whole.

### Definitions

Unless otherwise defined, all technical terms used herein have the same meaning as commonly understood by one of ordinary skill in the art.

The terminology used herein is for the purpose of describing particular examples only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The term “comprising” as used herein is synonymous with “including” or “containing”, and is inclusive or open-ended.

Any reference to “or” herein is intended to encompass “and/or” unless otherwise stated. As used herein, the term “about” a number refers to that number plus or minus 10% of that number. The term “about” a range refers to that range minus 10% of its lowest value and plus 10% of its greatest value. Reference to “about” a value or parameter herein includes (and describes) embodiments that are directed to that value or parameter per se.

As used herein, the terms “adeno-associated virus” and/or “AAV” refer to a parvovirus with a linear single-stranded DNA genome and variants thereof. The term covers all subtypes and both naturally occurring and recombinant forms, except where required otherwise.

The canonical AAV wild-type genome comprises approximately 4681 bases and includes terminal repeat sequences (e.g., inverted terminal repeats (ITRs)) at each end which function in cis as origins of DNA replication and as packaging signals for the virus. The genome includes two large open reading frames, known as AAV replication (“AAV rep” or “rep”) and capsid (“AAV cap” or “cap”) genes, respectively. AAV rep and cap may also be referred to herein as AAV “packaging genes.” These genes code for the viral proteins involved in replication and packaging of the viral genome.

An “AAV vector” or “rAAV vector” as used herein refers to an adeno-associated virus (AAV) or a recombinant AAV (rAAV) comprising a polynucleotide sequence not of AAV origin (e.g., a polynucleotide heterologous to AAV such as a nucleic acid sequence that encodes a therapeutic transgene, e.g., CNGB3) for transduction into a target cell or to a target tissue. In general, the heterologous polynucleotide is flanked by at least one, and generally by two, AAV inverted terminal repeat sequences (ITRs). The term rAAV vector encompasses both rAAV vector particles and rAAV vector plasmids. A rAAV vector may be either single-stranded (ssAAV) or self-complementary (scAAV).

An “AAV virus” or “AAV viral particle” or “rAAV vector particle” or “rAAV particle” refers to a viral particle comprising at least one AAV capsid protein and a polynucleotide rAAV vector. In some cases, the at least one AAV capsid protein is from a wild type AAV or is a variant AAV capsid protein (e.g., an AAV capsid protein with an insertion, e.g., an insertion of the 7m8 amino sequence as set forth below). If the particle comprises a heterologous polynucleotide (e.g., a polynucleotide other than a wild-type AAV genome such as a transgene to be delivered to a target cell or target tissue), it is referred to as a “rAAV particle”, “rAAV vector particle” or a “rAAV vector”. Thus, production of rAAV particles necessarily includes production of a rAAV vector, as such a vector contained within a rAAV particle. A rAAV may comprise an insertion in an AAV capsid that includes, but is not limited to, those disclosed in U.S. Pat. No. 9,193,956, WO2017197355, WO2018022905, WO2019104279 and/or US20210371879A1.

The term “packaging” as used herein can refer to a series of intracellular events that can result in the assembly and encapsidation of a rAAV particle.

AAV “rep” and “cap” genes refer to polynucleotide sequences encoding replication and encapsidation proteins of adeno-associated virus. AAV rep and cap are referred to herein as AAV “packaging genes.”

The genomic sequences of various serotypes of AAV, as well as the sequences of the inverted terminal repeats (ITRs), rep proteins, and capsid subunits are known in the art. Such sequences may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), AF028705.1 (AAV3B), NC_001829 (AAV 4), U89790 (AAV4), NC_006152 (AAV5), AF028704 (AAV6), AF513851 (AAV7), AF513852 (AAV8), NC_006261 (AAV8), AY530579 (AAV9), AY631965 (AAV10), AY631966 (AAV11), and DO813647 (AAV12); the disclosures of which are incorporated by reference herein. See also, e.g., Srivistava et al. (1983) J. Virology 45:555; Chiarini et al. (1998) J. Virology 71:6823; Chiarini et al. (1999) J. Virology 73:1309; Bantel-Schaal et al. (1999) J. Virology 73:939; Xiao et al. (1999) J. Virology 73:3994; Muramatsu et al. (1996) Virology 221:208; Shade et al. (1986) J. Viral. 58:921; Gao et al. (2002) Proc. Nat. Acad. Sci. USA 99:11854; Moris et al. (2004) Virology 33:375-383; international patent publications WO 00/28061, WO 99/61601, WO 98/11244; WO 2013/063379, WO 2014/194132, WO 2015/121501; and U.S. Pat. Nos. 6,156,303 and 7,906,111.

As used herein, the term “identity” or “identical to” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, 99% or more identical.

Calculation of the percent identity of two nucleic acid or polypeptide sequences, for example, can be performed by aligning two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of a reference sequence. Nucleotides at corresponding positions are then compared. When a position in a first sequence is occupied by the same residue (e.g., nucleotide or amino acid) as the corresponding position in a second sequence, then the molecules are identical at that position. The percent identity between two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm.

To determine percent identity, or homology, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA. Other techniques for alignment are described in Methods in Enzymology, vol. 266: Computer Methods for Macromolecular Sequence Analysis (1996), ed. Doolittle, Academic Press, Inc. Of particular interest are alignment programs that permit gaps in the sequence. Smith-Waterman is one type of algorithm that permits gaps in sequence alignments. See Meth. Mal. Biol. 70:173-187 (1997). Also, the GAP program using the Needleman and Wunsch alignment method can be utilized to align sequences. See J. Mal. Biol. 48:443-453 (1970).

Also of interest is the BestFit program using the local homology algorithm of Smith and Waterman (1981, Advances in Applied Mathematics 2:482-489) to determine sequence identity. The gap generation penalty will generally range from 1 to 5, usually 2 to 4 and in some embodiments will be 3. The gap extension penalty will generally range from about 0.01 to 0.20 and in some instances will be 0.10. The program has default parameters determined by the sequences inputted to be compared. Preferably, the sequence identity is determined using the default parameters determined by the program. This program is available also from Genetics Computing Group (GCG) package, from Madison, WI, USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

As used herein, the terms “inverted terminal repeat,” “ITR,” “terminal repeat,” and “TR” refer to palindromic terminal repeat sequences at or near the ends of the AAV virus genome, comprising mostly complementary, symmetrically arranged sequences. These ITRs can fold over to form T-shaped hairpin structures that function as primers during initiation of DNA replication. They are also needed for viral genome integration into host genome, for the rescue from the host genome; and for the encapsidation of viral nucleic acid into mature virions. The ITRs are required in cis for vector genome replication and its packaging into viral particles. “5′ ITR” refer to the ITR at the 5′ end of the AAV genome and/or 5′ to a recombinant transgene. “3′ ITR” refers to the ITR at the 3′ end of the AAV genome and/or 3′ to a recombinant transgene. Wild-type ITRs are approximately 145 bp in length. A modified, or recombinant ITR, may comprise a fragment or portion of a wild-type AAV ITR sequence. One of ordinary skill in the art will appreciate that during successive rounds of DNA replication ITR sequences may swap such that the 5′ ITR becomes the 3′ ITR, and vice versa. In some embodiments, at least one ITR is present at the 5′ and/or 3′ end of a recombinant vector genome such that the vector genome can be packaged into a capsid to produce a rAAV vector (also referred to herein as “rAAV vector particle” or “rAAV viral particle”) comprising the vector genome.

The ITRs are required in cis for vector genome replication and its packaging into viral particles. “5′ ITR” refers to the ITR at the 5′ end of the AAV genome and/or 5′ to a recombinant transgene. “3′ ITR” refers to the ITR at the 3′ end of the AAV genome and/or 3′ to a recombinant transgene. Wild-type ITRs are approximately 145 bp in length. A modified, or recombinant ITR, may comprise a fragment or portion of a wild-type AAV ITR sequence. One of ordinary skill in the art will appreciate that during successive rounds of DNA replication ITR sequences may swap such that the 5′ ITR becomes the 3′ ITR, and vice versa.

As used here, the term “nucleic acid construct,” refers to a non-naturally occurring nucleic acid molecule resulting from the use of recombinant DNA technology (e.g., a recombinant nucleic acid). A nucleic acid construct is a nucleic acid molecule, either single or double stranded, which has been modified to contain segments of nucleic acid sequences, which are combined and arranged in a manner not found in nature. A nucleic acid construct may be a “vector” (e.g., a plasmid, a rAAV vector genome, an expression vector, etc.), that is, a nucleic acid molecule designed to deliver exogenously created DNA into a host cell.

As used herein, the term “operably linked” refers to a linkage of nucleic acid sequence (or polypeptide) elements in a functional relationship. A nucleic acid is operably linked when it is placed into a functional relationship with another nucleic acid sequence. For instance, a promoter or other transcription regulatory sequence (e.g., an enhancer) is operably linked to a coding sequence if it affects the transcription of the coding sequence. In some embodiments, operably linked means that nucleic acid sequences being linked are contiguous. In some embodiments, operably linked does not mean that nucleic acid sequences are contiguously linked, rather intervening sequences are between those nucleic acid sequences that are linked.

As used herein, the terms “polypeptide,” “protein,” “peptide” or “encoded by a nucleic acid sequence” (i.e., encode by a polynucleotide sequence, encoded by a nucleotide sequence) refer to full-length native sequences, as with naturally occurring proteins, as well as functional subsequences, modified forms or sequence variants so long as the subsequence, modified form or variant retains some degree of functionality of the native full-length protein. In methods and uses of the disclosure, such polypeptides, proteins and peptides encoded by the nucleic acid sequences can be, but are not required to be, identical to the endogenous protein that is defective, or whose expression is insufficient, or deficient in a subject treated with gene therapy.

As used herein, the term “recombinant,” refers to a vector, polynucleotide (e.g., a recombinant nucleic acid), polypeptide or cell that is the product of various combinations of cloning, restriction or ligation steps (e.g., relating to a polynucleotide or polypeptide comprised therein), and/or other procedure that results in a construct that is distinct from a product found in nature. A recombinant virus or vector (e.g., rAAV vector) comprises a vector genome comprising a recombinant nucleic acid (e.g., a nucleic acid comprising a transgene and one or more regulatory elements). The terms respectively include replicates of the original polynucleotide construct and progeny of the original virus construct.

As used herein, the term “substantially” refers to the qualitative condition of exhibition of total or near-total extent or degree of a characteristic or property of interest. One of ordinary skill in the art will understand that biological and chemical phenomena rarely, if ever, go to completion and/or proceed to completeness or achieve or an absolute result. The term “substantially” is therefore used herein to capture the potential lack of completeness inherent in many biological and chemical phenomena.

As used herein, the term “therapeutic polypeptide” or “therapeutic protein” is a peptide, polypeptide or protein (e.g., enzyme, structural protein, transmembrane protein, transport protein) that may alleviate or reduce symptoms that result from an absence or defect in a protein in a target cell (e.g., an isolated cell) or organism (e.g., a subject). A therapeutic polypeptide or protein encoded by a transgene is one that confers a benefit to a subject, e.g., to correct a genetic defect, to correct a deficiency in a gene related to expression or function. Similarly, a “therapeutic transgene” is the transgene that encodes the therapeutic polypeptide. In some embodiments, a therapeutic polypeptide, expressed in a host cell, is an enzyme expressed from a transgene (i.e., an exogenous nucleic acid that has been introduced into the host cell). In some embodiments, a therapeutic polypeptide is a CNGB3 protein, or fragment thereof, expressed from a therapeutic transgene transduced into a retinal cell (e.g., a cone cell).

As used herein, the term “transgene” is used to mean any heterologous polynucleotide for delivery to and/or expression in a host cell, target cell or organism (e.g., a subject). Such “transgene” may be delivered to a host cell, target cell or organism using a vector (e.g., rAAV vector). A transgene may be operably linked to a control sequence, such as a promoter. It will be appreciated by those of skill in the art that expression control sequences can be selected based on an ability to promote expression of the transgene in a host cell, target cell or organism. Generally, a transgene may be operably linked to an endogenous promoter associated with the transgene in nature, but more typically, the transgene is operably linked to a promoter with which the transgene is not associated in nature. An example of a transgene is a nucleic acid encoding a therapeutic polypeptide, for example an CNGB3 polypeptide or fragment thereof, and an exemplary promoter is one not operable linked to a nucleotide encoding CNGB3 in nature. Such a nonendogenous promoter can include an OPN1LW promoter or a cone specific promoter, among many others known in the art.

A nucleic acid of interest can be introduced into a host cell by a wide variety of techniques that are well-known in the art, including transfection and transduction.

“Transfection” is generally known as a technique for introducing an exogenous nucleic acid into a cell without the use of a viral vector. As used herein, the term “transfection” refers to transfer of a recombinant nucleic acid (e.g., an expression plasmid) into a cell (e.g., a host cell) without use of a viral vector. A cell into which a recombinant nucleic acid has been introduced is referred to as a “transfected cell.” A transfected cell may be a host cell. The host cell may be a cell (e.g., a HEK293 cell or a sf9 cell) comprising an expression plasmid/vector for producing a recombinant AAV vector. In some embodiments, a transfected cell (e.g., a packing cell) may comprise a plasmid comprising a transgene (e.g., a CNGB3 transgene), a plasmid comprising an AAV rep gene and an AAV cap gene and a plasmid comprising a helper gene. Many transfection techniques are known in the art, which include, but are not limited to, electroporation, calcium phosphate precipitation, microinjection, cationic or anionic liposomes, and liposomes in combination with a nuclear localization signal.

As used herein, the term “transduction” refers to transfer of a nucleic acid (e.g., a vector genome) by a viral vector (e.g., rAAV vector) to a cell (e.g., a target cell, e.g., a retinal cell). In some embodiments, a gene therapy for Achromotopsia includes transducing a vector genome comprising a modified nucleic acid encoding CNGB3, or a fragment thereof, into a retinal cell. A cell into which a transgene has been introduced by a virus or a viral vector is referred to as a “transduced cell.” In some embodiments, a transduced cell is an isolated cell and transduction occurs ex vivo. In some embodiments, a transduced cell is a cell within an organism (e.g., a subject) and transduction occurs in vivo. A transduced cell may be a target cell of an organism which has been transduced by a recombinant AAV vector such that the target cell of the organism expresses a polynucleotide (e.g., a transgene, e.g., a modified nucleic acid encoding CNGB3, or a fragment thereof).

The term “subject”, “patient”, or “individual” refers to primates, such as humans and non-human primates, e.g., African green monkeys and rhesus monkeys. In some embodiments, the subject is a human.

The terms “treat,” “treating”, “treatment,” “ameliorate” or “ameliorating” and other grammatical equivalents as used herein, refer to alleviating, abating or ameliorating achromotopsia disease or disorder, or symptoms of achromotopsia disease or disorder, preventing additional symptoms of the achromotopsia disease or disorder, ameliorating or preventing the underlying causes of symptoms, inhibiting achromotopsia disease or disorder, e.g., arresting the development of achromotopsia disease or disorder, relieving achromotopsia disease or disorder, causing regression of achromotopsia disease or disorder, or stopping the symptoms of achromotopsia disease or disorder, and are intended to include prophylaxis. The terms further include achieving a therapeutic benefit and/or a prophylactic benefit. The term “therapeutic benefit” refers to eradication or amelioration of achromotopsia disease or disorder being treated. Also, a therapeutic benefit is achieved with the eradication or amelioration of one or more of the physiological symptoms associated with achromotopsia disease or disorder such that an improvement is observed in the subject, notwithstanding that, in some embodiments, the subject is still afflicted with achromotopsia disease or disorder. For prophylactic benefit, the pharmaceutical compositions are administered to a subject at risk of developing achromotopsia disease or disorder, or to a subject reporting one or more of the physiological symptoms of achromotopsia disease or disorder, even if a diagnosis of the disease or disorder has not been made.

The terms “administer,” “administering”, “administration,” and the like, as used herein, can refer to the methods that are used to enable delivery of therapeutics or pharmaceutical compositions to the desired site of biological action. These methods include intravitreal or subretinal injection to an eye.

The terms “effective amount”, “therapeutically effective amount” or “pharmaceutically effective amount” may be used interchangeably herein, and can refer to a sufficient amount of at least one pharmaceutical composition or compound being administered which will relieve to some extent one or more of the symptoms of the ocular disease or disorder being treated. An “effective amount”, “therapeutically effective amount” or “pharmaceutically effective amount” of a pharmaceutical composition may be administered to a subject in need thereof as a unit dose (as described in further detail elsewhere herein). In some embodiments, the term refers to an amount that is sufficient, when administered to a population suffering from or susceptible to a disease, disorder or condition in accordance with a therapeutic dosing regimen, to treat the disease, disorder or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment.

The term “pharmaceutically acceptable” as used herein, can refer to a material, such as a carrier or diluent, which does not abrogate the biological activity or properties of a compound disclosed herein, and is relatively nontoxic (i.e., when the material is administered to an individual it does not cause undesirable biological effects nor does it interact in a deleterious manner with any of the components of the composition in which it is contained).

The term “pharmaceutical composition,” or simply “composition” as used herein, can refer to a biologically active compound, optionally mixed with at least one pharmaceutically acceptable chemical component, such as, though not limited to carriers, stabilizers, diluents, dispersing agents, suspending agents, thickening agents, excipients and the like.

### 2. AAV and rAAV Vectors

### AAV

“Adeno-associated virus” and/or “AAV” refer to parvoviruses with a linear single-stranded DNA genome and variants thereof. The term covers all subtypes and both naturally occurring and recombinant forms, except where required otherwise. Parvoviruses, including AAV, are useful as gene therapy vectors as they can penetrate a cell and introduce a nucleic acid (e.g., transgene) into the nucleus. In some embodiments, the introduced nucleic acid (e.g., rAAV vector) forms circular concatemers that persist as episomes in the nucleus of transduced cells. In some embodiments, a transgene is inserted in specific sites in the host cell genome, for example at a site on human chromosome 8. Site-specific integration, as opposed to random integration, is believed to likely result in a predictable long-term expression profile. The insertion site of AAV into the human genome is referred to as AAVS1. Once introduced into a cell, polypeptides encoded by the nucleic acid can be expressed by the cell. Because AAV is not associated with any pathogenic disease in humans, a nucleic acid delivered by AAV can be used to express a therapeutic polypeptide for the treatment of a disease, disorder and/or condition in a human subject.

Genomic sequences of various serotypes of AAV, as well as sequences of the native terminal repeats (ITRs), rep proteins, and capsid subunits are known in the art. Such sequences may be found in the literature or in public databases such as Gen Bank. See, e.g., GenBank Accession Numbers NC_002077 (AAV1), AF063497 (AAV1), NC_001401 (AAV2), AF043303 (AAV2), NC_001729 (AAV3), AF028705.1 (AAV3B), NC_001829 (AAV4), U89790 (AAV4), NC_006152 (AAV5), AF028704 (AAV6), AF513851 (AAV7), AF513852 (AAV8), NC_006261 (AAV8), 5 AY530579 (AAV9), AY631965 (AAV10), AY631966 (AAV11), and DQ813647 (AAV12); the disclosures of which are incorporated by reference herein. See also, e.g., Srivistava et al. (1983) J. Virology 45:555; Chiarini et al. (1998) J. Virology 71:6823; Chiarini et al. (1999) J. Virology 73:1309; Bantel-Schaal et al. (1999) J. Virology 73:939; Xiao et al. (1999) J. Virology 73:3994; Muramatsu et al. (1996) Virology 221:208; Shade et al. (1986) J. Viral. 58:921; Gao et al. (2002) Proc. Nat. Acad. Sci. USA 99:11854; Moris et al. (2004) Virology 33:375-383; international patent publications WO 00/28061, WO 99/61601, WO 98/11244; WO 2013/063379; WO 2014/194132; WO 2015/121501, and U.S. Pat. Nos. 6,156,303 and 7,906,111. For illustrative purposes only, wild type AAV2 comprises a small (20-25 nm) icosahedral virus capsid of AAV composed of three proteins (VP1, VP2, and VP3; a total of 60 capsid proteins compose the AAV capsid) with overlapping sequences. The proteins VP1 (735 aa; Genbank Accession No. AAC03780), VP2 (598 aa; Genbank Accession No. AAC03778) and VP3 (533 aa; Genbank Accession No. AAC03779) exist in about a 1:1:10 ratio in the capsid. That is, for AAVs, VP1 is the full length protein and VP2 and VP3 are progressively shorter versions of VP1, with increasing truncation of the N-terminus relative to VP1. In one embodiment, of the method disclosed herein, a rAAV vector comprises an AAV2 VP1 comprising the amino acid sequence of SEQ ID NO:8.

### Recombinant AAV

As discussed supra, a “recombinant adeno-associated virus” or “rAAV” is distinguished from a wild-type AAV by replacement of all or part of the viral genome with a non-native sequence. Incorporation of a non-native sequence within the virus defines the viral vector as a “recombinant” vector, and hence a “rAAV vector” or “rAAV vector genome.” A rAAV vector can include a heterologous polynucleotide (e.g., human codon-optimized gene encoding human CNGB3, e.g., SEQ ID NO:20) encoding a desired protein or polypeptide (e.g., a CNGB3 polypeptide, or fragment thereof). A recombinant vector sequence may be encapsidated or packaged into an AAV capsid and referred to as an “rAAV vector,” an “rAAV vector particle,” “rAAV viral particle” or simply a “rAAV.”

The heterologous polynucleotide may be flanked by at least one, and sometimes by two, AAV terminal repeat sequences (e.g., inverted terminal repeats (ITRs)). The heterologous polynucleotide flanked by ITRs, also referred to herein as a “rAAV vector,” typically encodes a polypeptide of interest, or a gene of interest (“GOI”), such as a target for therapeutic treatment (e.g., a nucleic acid encoding CNGB3, or a fragment thereof, for the treatment of Achromotopsia). Delivery or administration of a rAAV vector to a subject (e.g., a patient) provides encoded proteins and peptides to the subject. Thus, a rAAV vector can be used to transfer/deliver a heterologous polynucleotide for expression for, e.g., treating a variety of diseases, disorders and conditions.

In some embodiments, a heterologous polypeptide comprises an ITR (e.g., an ITR from AAV2, but can comprise an ITR from any wild type AAV serotype, or a variant thereof) positioned at the left and right ends (i.e., 5′ and 3′ termini, respectively) of a vector genome. In some embodiments, a left (e.g., 5′) ITR comprises or consists of the nucleic acid sequence of SEQ ID NO: 12. In some embodiments, a left (e.g., 5′) ITR comprises a nucleic acid sequence that is about 80%, about 85%, about 90%, about 95%, about 98%, about 99% or 100% identical to SEQ ID NO: 12. In some embodiments, a right (e.g., 3′) ITR comprises or consists of a nucleic acid sequence of SEQ ID NO: 12. In some embodiments, a right (e.g., 3′) ITR comprises a nucleic acid sequence that is about 80%, about 85%, about 90%, about 95%, about 98%, about 99% or 100% identical to SEQ ID NO: 12. Each ITR is in cis with but may be separated from each other, or other elements in the vector genome, by a nucleic acid sequence of variable length, such as a recombinant nucleic acid comprising a modified nucleic acid encoding CNGB3, and regulatory elements. In some embodiments, ITRs are AAV2 ITRs, or variants thereof, and flank a CNGB3 transgene. In some embodiments, a rAAV comprises a CNGB3 transgene (e.g., comprising the nucleic acid sequence of SEQ ID NO:20) flanked by AAV2 ITRs (e.g., ITRs having the sequence as set forth in SEQ ID NO: 12).

In some embodiments, a rAAV vector genome is linear, single-stranded and flanked by AAV ITRs. Prior to transcription and translation of the heterologous gene, a single stranded DNA genome of approximately 4700 nucleotides must be converted to a double-stranded form by DNA polymerases (e.g., DNA polymerases within the transduced cell) using the free 3′-OH of one of the self-priming ITRs to initiate second-strand synthesis. In some embodiments, full length-single stranded vector genomes (i.e., sense and anti-sense) anneal to generate a full length-double stranded vector genome. This may occur when multiple rAAV vectors carrying genomes of opposite polarity (i.e., sense or anti-sense) simultaneously transduce the same cell. Regardless of how they are produced, once double-stranded vector genomes are formed, the cell can transcribe and translate the double-stranded DNA and express the heterologous gene.

The efficiency of transgene expression from a rAAV vector can be hindered by the need to convert a single stranded rAAV genome (ssAAV) into double-stranded DNA prior to expression. This step is circumvented by using a self-complementary AAV genome (scAAV) that can package an inverted repeat genome that can fold into double-stranded DNA without the need for DNA synthesis or basepairing between multiple vector genomes (McCarty, (2008) Malec. Therapy 16 (10): 1648-1656; McCarty et al., (2001) Gene Therapy 8:1248-1254; McCarty et al., (2003) Gene Therapy 10:2112-2118). A limitation of a scAAV vector is that size of the unique transgene, regulatory elements and IRTs to be package in the capsid is about half the size (i.e., ˜2,500 nucleotides of which 2,200 nucleotides may be a transgene and regulatory elements, plus two copies of the ˜ 145 nucleotide ITRs) of a ssAAV vector genome (i.e., ˜4,900 nucleotides including two ITRs).

scAAV vector genomes are made by deleting the terminal resolution site (TRS) from one rAAV ITR of the expression plasmid, thereby preventing initiation of replication from that end (see U.S. Pat. No. 8,784,799). AAV replication within a host cell is initiated at the wild type ITR of the genome and continues through the mutant ITR without terminal resolution and then back across the genome to create a dimer. The dimer is a self-complementary genome with a mutant ITR in the middle, and wild-type ITRs at each end. In some embodiments, a mutant ITR with a deleted TRS is at the 5′ end of the vector genome. In some embodiments, a mutant ITR with a deleted TRS is at the 3′ end of the vector genome.

Without wishing to be bound by theory, while the two halves of a scAAV genome are complementary, it is unlikely that there is substantial base pairing within the capsid as many of the bases are in contact with amino acid residues of the inner capsid shell and the phosphate backbone is sequestered toward the center (McCarty, Malec. Therapy (2008) 16 (10): 1648-1656). It likely that upon uncoating, the two halves of the scAAV genome anneal to form a dsDNA hairpin molecule, with a covalently closed ITR at one end and two open-ended ITRs on the other. The ITRs flank a double-stranded region encoding, among other things, the transgene, and regulatory elements in cis thereto.

A viral capsid of a rAAV vector may be from a wild type AAV or a variant AAV such as AAV1, AAV2, AAV2.7m8, AAV2.5T, AAV2.LSV1 (PCT/US2020/029895), AAV3, AAV3A, AAV3B, AAV4, AAV5, AAV6, AAV7, AAV8, AAV9, AAV10, AAVrh10, AAVrh74 (see WO2016/210170), AAV12, AAV2i8, AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAVhu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAV avian AAV, bovine AAV, canine AAV, equine AAV, primate AAV, non-primate AAV, snake AAV, goat AAV, shrimp AAV, ovine AAV and variants thereof (see, e.g., Fields et al., VIROLOGY, volume 2, chapter 69 (4th ed., Lippincott-Raven Publishers). Capsids may be derived from a number of AAV serotypes disclosed in U.S. Pat. No. 7,906,111; Gao et al. (2004) J. Viral. 78:6381; Morris et al. (2004) Viral. 33:375; WO 2013/063379; WO 2014/194132; and include true type AAV (AAV-TT) variants disclosed in WO 2015/121501, and RHM4-1, RHM15-1 through RHM15-6, and variants thereof, disclosed in WO 2015/013313. Capsids may also be derived from AAV variants isolated from human CD34+ cell include AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15 (Smith et al. (2014) Molecular Therapy 22 (9): 1625-1634). One skilled in the art would know there are likely other AAV variants not yet identified that perform the same or similar function. A full complement of AAV cap proteins includes VP1, VP2, and VP3. The ORF comprising nucleotide sequences encoding AAV VP capsid proteins may comprise less than a full complement AAV Cap proteins or the full complement of AAV cap proteins may be provided.

In some embodiments, chimeric vectors have been engineered to exhibit altered tropism or tropism for a particular tissue or cell type. The term “tropism” refers to preferential entry of the virus into certain cell or tissue types and/or preferential interaction with the cell surface that facilitates entry into certain cell or tissue types. AAV tropism is generally determined by the specific interaction between distinct viral capsid proteins and their cognate cellular receptors (Lykken et al. (2018) J. Neurodev. Disord. 10:16). Preferably, once a virus or viral vector has entered a cell, sequences (e.g., heterologous sequences such as a transgene) carried by the vector genome (e.g., a rAAV vector genome) are expressed.

A “tropism profile” refers to a pattern of transduction of one or more target cells, tissues and/or organs. For example, an AAV capsid may have a tropism profile characterized by efficient transduction of retinal cells (e.g., cone cells) with only low transduction of, for example, heart cells.

### 3. Recombinant Nucleic Acids

Methods of the present disclosure include purification of a rAAV vector comprising a recombinant nucleic acid including modified nucleic acids as well as plasmids and vector genomes that comprise a modified nucleic acid. A recombinant nucleic acid, plasmid or vector genome may comprise regulatory sequences to modulate propagation (e.g., of a plasmid) and/or control expression of a modified nucleic acid (e.g., a transgene). Recombinant nucleic acids may also be provided as a component of a viral vector (e.g., a rAAV vector). Generally, a viral vector includes a vector genome comprising a recombinant nucleic acid packaged in a capsid.

### Modified Nucleic Acids

A modified, or variant form, of a gene, nucleic acid or polynucleotide (e.g., a transgene) refers to a nucleic acid that deviates from a reference sequence. A reference sequence may be a naturally occurring, wild type sequence (e.g., a gene) and may include naturally occurring variants (e.g., splice variants, alternative reading frames). Those skilled in the art will be aware that reference sequences can be found in publicly available databases such as GenBank (ncbi.nlm.nih.gov/genbank). Modified or variant nucleic acids may have substantially the same, greater or lesser activity, function or expression as compared to a reference sequence. Preferably, a modified, or variant nucleic acid, as used interchangeably herein, exhibits improved protein expression, e.g., a protein encoded thereby is expressed at a detectably greater level in a cell compared with the level of expression of a protein provided by an endogenous gene (e.g., a wild type gene, a mutant gene) in an otherwise identical cell. In some embodiments, a modified, or variant nucleic acid, as used interchangeably herein, exhibits improved protein expression, e.g., a protein encoded thereby is expressed at a detectably greater level in a cell compared with the level of expression of a protein provided by an endogenous gene comprising a mutation in an otherwise identical cell.

Modifications to nucleic acids include one or more nucleotide substitutions (e.g., substitution of 1-3, 3-5, 5-10, 10-15, 15-20, 20-25, 25-30, 30-40, 40-50, 50-100 or more nucleotides), additions (e.g., insertion of 1-3, 3-5, 5-10, 10-15, 15-20, 20-25, 25-30, 30-40, 40-50, 50-100 or more nucleotides), deletions (e.g., deletion of 1-3, 3-5, 5-10, 10-15, 15-20, 20-25, 25-30, 30-40, 40-50, 50-100 or more nucleotides, deletion of a motif, domain, fragment, etc.) of a reference sequence. A modified nucleic acid may be about 50%, about 60%, about 70%, about 80%, about 85%, about 90%, about 92%, about 93%, about 94%, about 95%, about 96% about 97% about 98% or about 99% identical to a reference sequence.

A modified nucleic acid may encode a polypeptide with about 50%, about 60%, about 70%, about 80%, about 85%, about 90%, about 95%, about 98%, about 99% or 100% identity to a reference polypeptide. In some embodiments, a modified nucleic acid encodes a polypeptide with 100% identify to a reference polypeptide.

In some embodiments, a modified nucleic acid (e.g., transgene) encodes a wild-type protein (e.g., a CNGB3 polypeptide, e.g., SEQ ID NO:11). Such modified nucleic acid may be codon optimized. “Optimized” or “codon-optimized,” as referred to interchangeably herein, refers to a coding sequence that has been optimized relative to a wild type coding sequence or reference sequence (e.g., a coding sequence for a CNGB3 polypeptide, e.g., SEQ ID NO: 10) to increase expression of the polypeptide, e.g., by minimizing usage of rare codons, decreasing the number of CpG di-nucleotides, removing cryptic splice donor or acceptor sites, removing Kozak sequences, removing ribosomal entry sites, and the like. In some embodiments, a level of expression of a protein from a codon-optimized sequence is increased as compared to a level of expression of a protein from a wild type gene in an otherwise identical cell. In some embodiments, a level of expression of a protein from a codon-optimized sequence is not increased (e.g., expression is substantially similar) as compared to a level of expression of a protein from a wild-type gene in an otherwise identical cell. In some embodiments, a level of expression of a protein from a codon-optimized sequence is increased as compared to a level of expression of a protein from a mutant gene in an otherwise identical cell.

Examples of modifications include elimination of one or more cis-acting motifs and introduction of one or more Kozak sequences. In some embodiments, one or more cis-acting motifs are eliminated and one or more Kozak sequences are introduced. In some embodiments, the Kozak sequence has been optimized (e.g., SEQ ID NO: 19).

Examples of cis-acting motifs that may be eliminated include internal TATA-boxes; chi-sites; ribosomal entry sites; ARE, INS, and/or CRS sequence elements; repeat sequences and/or RNA secondary structures; (cryptic) splice donor and/or acceptor sites, branch points; and restriction sites.

In some embodiments, a modified nucleic acid encodes a modified or variant polypeptide. A modified polypeptide encoded by a modified nucleic acid (e.g., a codon optimized CNGB3 gene) may retain all or a part of the function or activity of a polypeptide encoded by a wild type coding or reference sequence. In some embodiments, a modified polypeptide has one or more non-conservative or conservative amino acid changes. In some embodiments, certain domains that have been demonstrated to play a limited or no role in a function of a polypeptide are not present in a modified polypeptide (e.g., certain binding domains). Modified nucleic acids present in rAAV vectors may comprise fewer nucleotides than the wild type coding, or reference sequence, due to the packaging capacity of a rAAV capsid (e.g., shortened CNGB3 transgene), and also include shortened transgenes that are both truncated and codon-optimized (e.g., a codon optimized CNGB3 transgene described herein). In some embodiments, a polypeptide encoded by a modified nucleic acid has less than, the same, or greater, but at least a part of, a function or activity of a polypeptide encoded by a reference sequence.

Modified nucleic acids may have a modified GC content (e.g., the number of G and C nucleotides present in a nucleic acid sequence), a modified (e.g., increased or decreased) CpG dinucleotide content and/or a modified (e.g., increased or decreased) codon adaptation index (CAI) relative to a reference and/or wild-type sequence. See, e.g., WO 2017/077451 (discussing various considerations well-known in the art for codon-optimization of nucleic acid sequences of interest). As used herein, modified refers to a decrease or an increase in a particular value, amount or effect.

In some embodiments, a GC content of a modified nucleic acid sequence of the present disclosure may increase or decrease relative to a reference and/or a wild-type gene or coding sequence during the codon optimization process. In some embodiments the GC content is less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% of the codon optimized coding sequence. In some embodiments, GC content is expressed as a percentage of G (guanine) and C (cytosine) nucleotides in the sequence. In some embodiments, a modified nucleic acid has 1-5 fewer, 5-10 fewer, 10-15 fewer, 15-20 fewer, 20-25 fewer, 25-30 fewer, 30-40 fewer, 40-45 fewer or 45-50 fewer, or even fewer CpG di-nucleotides than a reference sequence (e.g., a wild type sequence).

In some embodiments, a codon adaptation index of a modified nucleic acid sequence of the present disclosure is at least 0.74, at least 0.76, at least 0.77, at least 0.80, at least 0.85, at least 0.86, at least 0.87, at least 0.90, at least 0.95 or at least 0.98.

Modified nucleic acid sequences may include flanking restriction sites to facilitate subcloning into an expression vector. Many such restriction sites are well known in the art, and include, but are not limited to, Aval, NotI, SpeI, NheI, SnaBI, AvrII, BamHI, HindIII, EcoRI, EcoRV, SacI, SwaI, ApaI 1 and XmaI.

The present disclosure includes a modified nucleic acid of SEQ ID NO:20 which encodes a functionally active CNGB3 polypeptide. A “functionally active” or “functional CNGB3 polypeptide” indicates that the protein provides the same or similar biological function and/or activity as a wild-type CNGB3 polypeptide.

Thus, one embodiment of the invention relates to a method of purifying a rAAV vector comprising a modified nucleic acid encoding a CNGB3 protein, the nucleic acid comprising, consisting essentially of, or consisting of the nucleic acid sequence of SEQ ID NO:20 or a sequence at least about 90% identical thereto. In some embodiments, the nucleic acid is at least about 90%, 91%, 92%, 93%, 94%, 95%, 96%, 97%, 98%, or 99% identical to the nucleic acid sequence of SEQ ID NO:20. In certain embodiments, the nucleic acid has a length that is within the capacity of a viral vector, e.g., a parvovirus vector, e.g., a rAAV vector. In some embodiments, the nucleic acid is about 5000, 4900, 4800, 4700, 4600, 4500, 4400, 4300, 4200, 4100, or about 4000 nucleotides, or fewer.

In some embodiments, a nucleic acid encodes a CNGB3 protein comprising, consisting essentially of, or consisting of the amino acid sequence of SEQ ID NO:11 or a sequence at least about 90%, 91%, 92%, 93%, 94%, 95%, 96%, 97%, 98%, or 99% identical to the amino acid sequence of SEQ ID NO: 11.

In some embodiments, a nucleic acid (e.g., SEQ ID NO:20) is part of a recombinant nucleic acid for production of CNGB3 protein. The recombinant nucleic acid may further comprise regulatory elements useful for increasing expression of CNGB3.

### Regulatory Elements

Methods of the present disclosure include purification of a rAAV vector comprising a recombinant nucleic acid including a modified nucleic acid encoding a polypeptide (e.g., CNGB3) and various regulatory or control elements. Typically, regulatory elements are nucleic acid sequence(s) that influence expression of an operably linked polynucleotide. The precise nature of regulatory elements useful for gene expression will vary from organism to organism and from cell type to cell type including, for example, a promoter, enhancer, intron etc., with the intent to facilitate proper heterologous polynucleotide transcription and translation. Regulatory control can be affected at the level of transcription, translation, splicing, message stability, etc. Typically, a regulatory control element that modulates transcription is juxtaposed near the 5′ end of the transcribed polynucleotide (i.e., upstream). Regulatory control elements may also be located at the 3′ end of the transcribed sequence (i.e., downstream) or within the transcript (e.g., in an intron). Regulatory control elements can be located at a distance away from the transcribed sequence (e.g., 1 to 100, 100 to 500, 500 to 1000, 1000 to 5000, 5000 to 10,000 or more nucleotides). However, due to the length of an AAV vector genome, regulatory control elements are typically within 1 to 1000 nucleotides from the polynucleotide.

In some embodiments, a part of the MNTC regulatory elements (i.e., LCR enhancer (SEQ ID NO: 13), core promoter (SEQ ID NO: 15) and chimeric intron (SEQ ID NO: 16) are used.

### Promoter

As used herein, the term “promoter” refers to a nucleotide sequence that initiates transcription of a particular gene, or one or more coding sequences (e.g., an CNGB3 coding sequence), in eukaryotic cells (e.g., a cone cell). A promoter can work with other regulatory elements or regions to direct the level of transcription of the gene or coding sequence(s). These regulatory elements include, for example, transcription binding sites, repressor and activator protein binding sites, and other nucleotide sequences known to act directly or indirectly to regulate the amount of transcription from the promoter, including, for example, attenuators, enhances and silencers. The promoter is most often located on the same strand and near the transcription start site, 5′ of the gene or coding sequence to which it is operably linked. A promoter is generally 100-1000 nucleotides in length. A promoter typically increases gene expression relative to expression of the same gene in the absence of a promoter.

As used herein, a “core promoter” or “minimal promoter” refers to the minimal portion of a promoter sequence required to properly initiate transcription. See, for example, WO2015/142941. It may include any of the following: a transcription start site, a binding site for RNA polymerase and a general transcription factor binding site. A promoter may also comprise a proximal promoter sequence (5′ of a core promoter) that contains other primary regulatory elements (e.g., enhancer, silencer, boundary element, insulator) as well as a distal promoter sequence (3′ of a core promoter).

In some embodiments, the promoter is a promoter comprising, consisting essentially of, or consisting of the nucleotide sequence of SEQ ID NO: 14 or SEQ ID NO: 15.

In some embodiments of the present disclosure, a promoter sequence (e.g., SEQ ID NO: 14 or SEQ ID NO: 15) is operably linked to a modified nucleic acid encoding CNGB3 (e.g., SEQ ID NO: 10 or SEQ ID NO:20). In some embodiments, a promoter comprising the nucleic acid sequence of SEQ ID NO: 14 or SEQ ID NO: 15 is operably linked to a modified nucleic acid encoding CNGB3 (e.g., SEQ ID NO:20). In some embodiments, a promoter comprising a nucleic acid sequence at least 80%, at least 85%, at least 90%, at least 95%, at least 98%, at least 99% or 100% identical to the nucleic acid sequence of SEQ ID NO: 14 is operably linked to a nucleic acid comprising the nucleic acid sequence of SEQ ID NO: 10 or a sequence at least 80%, at least 85%, at least 90%, at least 95%, at least 98%, at least 99% or 100% identical thereto. In some embodiments, a promoter comprising a nucleic acid sequence at least 80%, at least 85%, at least 90%, at least 95%, at least 98%, at least 99% or 100% identical to the nucleic acid sequence of SEQ ID NO: 14 is operably linked to a nucleic acid comprising the nucleic acid sequence of SEQ ID NO:20 or a sequence at least 80%, at least 85%, at least 90%, at least 95%, at least 98%, at least 99% or 100% identical thereto. In some embodiments, a promoter comprising a nucleic acid sequence at least 80%, at least 85%, at least 90%, at least 95%, at least 98%, at least 99% or 100% identical to the nucleic acid sequence of SEQ ID NO: 15 is operably linked to a nucleic acid comprising the nucleic acid sequence of SEQ ID NO: 10 or a sequence at least 80%, at least 85%, at least 90%, at least 95%, at least 98%, at least 99% or 100% identical thereto. In some embodiments, a promoter comprising a nucleic acid sequence at least 80%, at least 85%, at least 90%, at least 95%, at least 98%, at least 99% or 100% identical to the nucleic acid sequence of SEQ ID NO: 15 is operably linked to a nucleic acid comprising the nucleic acid sequence of SEQ ID NO:20 or a sequence at least 80%, at least 85%, at least 90%, at least 95%, at least 98%, at least 99% or 100% identical thereto. In some embodiments, a promoter comprising a nucleic acid sequence at least 80%, at least 85%, at least 90%, at least 95%, at least 98%, at least 99% or 100% identical to the nucleic acid sequence of either SEQ ID NO: 14 or SEQ ID NO: 15 is operably linked to a nucleic acid encoding an amino acid sequence according to SEQ ID NO: 11.

In some embodiments, a promoter comprising a nucleic acid sequence at least 95% identical to the nucleic acid sequence of SEQ ID NO: 14 or SEQ ID NO: 15 is operably linked to a nucleic acid sequence at least 95% identical to the nucleic acid sequence of SEQ ID NO:20 and induces expression of a polypeptide of SEQ ID NO: 11 in cone cells.

A promoter may be constitutive, tissue-specific or regulated. Constitutive promoters are those which cause an operably linked gene to be expressed essentially at all times. In some embodiments, a constitutive promoter is active in most eukaryotic tissues under most physiological and developmental conditions.

Regulated promoters are those which can be activated or deactivated. Regulated promoters include inducible promoters, which are usually “off,” but which may be induced to turn “on,” and “repressible” promoters, which are usually “on,” but may be turned “off.” Many different regulators are known, including temperature, hormones, cytokines, heavy metals and regulatory proteins. The distinctions are not absolute; a constitutive promoter may often be regulated to some degree. In some cases, an endogenous pathway may be utilized to provide regulation of the transgene expression, e.g., using a promoter that is naturally downregulated when the pathological condition improves.

A tissue-specific promoter is a promoter that is active in only specific types of tissues, cells or organs. Typically, a tissue-specific promoter is recognized by transcriptional activator elements that are specific to a particular tissue, cell and/or organ. For example, a tissue-specific promoter may be more active in one or several particular tissues (e.g., two, three or four) than in other tissues. In some embodiments, expression of a gene modulated by a tissue-specific promoter is much higher in the tissue for which the promoter is specific than in other tissues. In some embodiments, there may be little, or substantially no activity, of the promoter in any tissue other than the one for which it is specific. A promoter may be a tissue-specific promoter, such as the mouse albumin promoter, or the transthyretin promoter (TTR) which are active in liver cells. Other examples of tissue specific promoters include promoters from genes encoding skeletal a-actin, myosin light chain 2A, CNGB3, muscle creatine kinase which induce expression in skeletal muscle (Li et al. (1999) Nat. Biotech. 17:241-245).

### Enhancer

In another aspect, a modified nucleic acid encoding a therapeutic polypeptide further comprises an enhancer to increase expression of the therapeutic polypeptide. Typically, an enhancer element is located upstream of a promoter element but may also be located downstream or within another sequence (e.g., a transgene). An enhancer may be located 0-100 nucleotides, 200 nucleotides, 300 nucleotides or more upstream or downstream of a modified nucleic acid. An enhancer typically increases expression of a modified nucleic acid (e.g., encoding a therapeutic polypeptide) beyond the increased expression provided by a promoter element alone.

Many enhancers are known in the art, including, but not limited to, the cytomegalovirus major immediate-early enhancer. More specifically, the cytomegalovirus (CMV) MIE promoter comprises three regions: the modulator, the unique region and the enhancer (Isomura and Stinski (2003) J. Viral. 77 (6): 3602-3614). The CMV enhancer region can be combined with another promoter, or a portion thereof, to form a hybrid promoter to further increase expression of a nucleic acid operably linked thereto. For example, a chicken beta-actin (CBA) promoter, or a portion thereof, can be combined with a CMV promoter/enhancer, or a portion thereof, to make a version of CBA termed the “CBh” promoter, which stands for chicken beta-actin hybrid promoter, as described in Gray et al. (2011, Human Gene Therapy 22:1143-1153). Like promoters, enhancers may be constitutive, tissue specific or regulated.

In some embodiments of the present disclosure, an enhancer comprising SEQ ID NO: 13 is operably linked to a modified nucleic acid encoding CNGB3. In some embodiments, the enhancer, e.g., SEQ ID NO:13, is upstream of the promoter.

### Fillers, Spacers and Stuffers

As disclosed herein, a recombinant nucleic acid intended for use in a rAAV vector may include an additional nucleic acid element to adjust the length of the nucleic acid to near, or at the normal size (e.g., approximately 4.7 to 4.9 kilobases), of the viral genomic sequence acceptable for AAV packaging into a rAAV vector (Grieger and Samulski (2005) J. Viral. 79 (15): 9933-9944). Such a sequence may be referred to interchangeably as filler, spacer or stuffer. In some embodiments, filler DNA is an untranslated (non-protein coding) segment of nucleic acid. In some embodiments, a filler or stuffer polynucleotide sequence is a sequence between about 1-10, 10-20, 20-30, 30-40, 40-50, 50-60, 60-70, 70-80, 80-90-90-100, 100-150, 150-200, 200-250, 250-300, 300-400, 400-500, 500-750, 750-1000, 1000-1500, 1500-2000, 2000-3000 or more in length.

AAV vectors typically accept inserts of DNA having a size ranging from about 4 kb to about 5.2 kb or about 4.1 to 4.9 kb for optimal packaging of the nucleic acid into the AAV capsid. In some embodiments, a rAAV vector comprises a vector genome having a total length between about 4.0 kb to about 4.5 kb, about 4.5 kb to about 5.0 kb or about 5.0 kb to about 5.2 kb. In some embodiments, a rAAV vector comprises a vector genome having a total length of about 4.5 kb. In some embodiments, a rAAV vector comprises a vector genome that is self-complementary. While the total length of a self-complementary (sc) vector genome in a rAAV vector is equivalent to a single stranded (ss) vector genome (i.e., from about 4 kb to about 5.2 kb), the nucleic acid sequence (i.e., comprising the transgene, regulatory elements and ITRs) encoding the sc vector genome must be only half as long as a nucleic acid sequence encoding a ss vector genome in order for the sc vector genome to be packaged in the capsid.

### Introns and Exons

In some embodiments, a recombinant nucleic acid includes, for example, an intron, exon and/or a portion thereof. An intron may function as a filler or stuffer polynucleotide sequence to achieve an appropriate length for vector genome packaging into a rAAV vector. An intron and/or an exon sequence can also enhance expression of a polypeptide (e.g., a transgene) as compared to expression in the absence of the intron and/or exon element.

An intron element may be derived from the same gene as a heterologous polynucleotide, or derived from a completely different gene or other DNA sequence (e.g., chicken beta-actin gene, minute virus of mice (MVM)). In some embodiments, a recombinant nucleic acid includes at least one intron. In some embodiments, the intron comprises, consists of, consists essentially of SEQ ID NO: 16.

Polyadenylation Signal Sequence (polyA)

Further regulatory elements may include a stop codon, a termination sequence, and a polyadenylation (polyA) signal sequence, such as, but not limited to the short synthetic polyadenylation sequence (SPA) as provided herein. A polyA signal sequence drives efficient addition of a poly-adenosine “tail” at the 3′ end of a eukaryotic mRNA which guides termination of gene transcription. A polyA signal acts as a signal for the endonucleolytic cleavage of the newly formed precursor mRNA at its 3′ end and for addition to this 3′ end of an RNA stretch consisting only of adenine bases. A polyA tail is important for the nuclear export, translation and stability of mRNA. In some embodiments, a poly A is a SV40 early polyadenylation signal, a SV40 late polyadenylation signal, a short synthetic polyadenylation sequence, an HSV thymidine kinase polyadenylation signal, a protamine gene polyadenylation signal, an adenovirus 5 E1b polyadenylation signal, a growth hormone polyadenylation signal, a PBGD polyadenylation signal or an in silica designed polyadenylation signal.

In some embodiments, and optionally in combination with one or more other regulatory elements described herein, a polyA signal sequence of a recombinant nucleic acid is a polyA signal that is capable of directing and effecting the endonucleolytic cleavage and polyadenylation of the precursor mRNA resulting from the transcription of a modified nucleic acid encoding e.g., CNGB3 (e.g., SEQ ID NO: 11). In some embodiments, a polyA sequence comprises or consists of the nucleic acid sequence of SEQ ID NO:22 or SEQ ID NO:23. In some embodiments, a polyA sequence comprises a nucleic acid sequence about 80%, about 85%, about 90%, about 95%, about 98%, about 99% or 100% identical to the nucleic acid sequence of SEQ ID NO:22 or SEQ ID NO:23.

In some embodiments, a recombinant nucleic acid comprises at least one of: a promoter sequence (e.g., SEQ ID NO:14, SEQ ID NO:15), an enhancer and promoter (e.g., SEQ ID NOs: 13 and 15) and a polyA (SEQ ID NO:23) and modulates the expression of a heterologous polypeptide, optionally encoded by the nucleic acid sequence of SEQ ID NO: 10 or SEQ ID NO:20 or nucleotides 2122-4551 of SEQ ID NO:2.

In some embodiments, a recombinant nucleic acid comprises a promoter sequence (e.g., SEQ ID NO: 14), and a polyA (SEQ ID NO:22) and modulates the expression of a heterologous polypeptide comprising the amino acid sequence of SEQ ID NO: 20.

In some embodiments, a rAAV2 vector with tropism for cone cells, contains a vector genome comprising AAV ITRs (e.g., AAV2 ITRs) and a recombinant nucleic acid comprising a modified (i.e., codon-optimized) nucleic acid encoding CNGB3 and at least one of the following regulatory elements: a promoter (e.g., SEQ ID NO: 13 or 14), an enhancer (SEQ ID NO: 13) and a poly A signal sequence. The polyA signal is either SEQ ID NO: 22 or SEQ ID NO:23.

In some embodiments, a rAAV2 vector with tropism for cone cells, contains a vector genome comprising AAV ITRs (e.g., AAV2 ITRs) and a recombinant nucleic acid comprising a modified (i.e., codon-optimized) nucleic acid encoding CNGB3 and the following regulatory elements: an enhancer, a promoter (e.g., SEQ ID NO: 13), an intron (e.g., SEQ ID NO: 16) and a poly A signal sequence. The polyA signal is either SEQ ID NO: 22 or SEQ ID NO:23.

In some embodiments, a rAAV2 vector with tropism for cone cells, contains a vector genome comprising AAV ITRs (e.g., AAV2 ITRs) and a recombinant nucleic acid comprising a modified (i.e., codon-optimized) nucleic acid encoding CNGB3 and the following regulatory elements: an enhancer, a promoter (e.g., SEQ ID NO: 13), an intron (e.g., SEQ ID NO: 16), an optimized Kozak Sequence (SEQ ID NO:19), and a poly A signal sequence, further optionally at least one of the following: (i) an optimized 10 nt optimized lead sequence upstream of the optimized Kozak sequence, and (ii) a 5′UTR. The 5′ UTR is selected from (a) the partial human CTNNB1 5′UTR sequence (SEQ ID NO: 18), (b) the partial human TUBB 5′UTR sequence (SEQ ID NO:25), and (c) SEQ ID NO:17. The polyA signal is either SEQ ID NO:22 or SEQ ID NO:23.

### 4. Pharmaceutical Compositions

In some embodiments, provided is a pharmaceutical composition comprising:
a) a nucleic acid comprising a nucleotide sequence that encodes a CNGB3 protein as provided for herein, an expression vector described herein, or a virion comprising a nucleic acid or expression vector described herein, and b) a pharmaceutically acceptable excipient or carrier. In some embodiments “pharmacologically acceptable excipient or carrier” refers to any excipient, carrier, diluent, stabilizer, etc., that has substantially no long-term or permanent detrimental effect when administered to a subject (e.g., a mammal, such as a mouse, a human, or a non-human primate). Typically, such excipient is mixed with an active compound (e.g., a nucleic acid disclosed herein, an expression vector disclosed herein, or a viral particle disclosed herein), or permitted to dilute or enclose the active compound and can be a solid, semi-solid, or liquid agent. It is understood that the active ingredients can be soluble or can be delivered as a suspension in the desired excipient or diluent. Any of a variety of pharmaceutically acceptable excipients can be used including, without limitation, aqueous media such as, e.g., distilled, deionized water, saline; solvents; dispersion media; coatings; antibacterial and antifungal agents; isotonic and absorption delaying agents; or any other inactive ingredient. Selection of a pharmacologically acceptable excipient can depend on the mode of administration. Except insofar as any pharmacologically acceptable excipient is incompatible with the active ingredient, its use in pharmaceutically acceptable compositions is contemplated. Some examples of materials that can serve as pharmaceutically-acceptable excipients include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and other waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations. Other non-limiting examples of specific uses of such pharmaceutical carriers can be found in “Pharmaceutical Dosage Forms and Drug Delivery Systems” (Howard C. Ansel et al., eds., Lippincott Williams & Wilkins Publishers, 7th ed. 1999); “Remington: The Science and Practice of Pharmacy” (Alfonso R. Gennaro ed., Lippincott, Williams & Wilkins, 20th 2000); “Goodman & Gilman's The Pharmacological Basis of Therapeutics 13th ed.” Brunton et al., eds., McGraw-Hill Professional, 2017); and “Handbook of Pharmaceutical Excipients” (Sheskey et al., APhA Publications, 9th edition 2020).

In some embodiments, the pharmaceutical composition further comprises one or more additional pharmaceutically acceptable component(s), e.g., buffers, preservatives, tonicity adjusters, salts, antioxidants, physiological substances, pharmacological substances, bulking agents, emulsifying agents, wetting agents, and the like. Various buffers and means for adjusting pH can be used to prepare a pharmaceutical composition, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate and a stabilized oxy chloro composition, for example, PURITE™. Tonicity adjustors suitable for inclusion in a pharmaceutical composition include, without limitation, salts such as, e.g., sodium chloride, potassium chloride, mannitol or glycerin and other pharmaceutically acceptable tonicity adjustor. It is understood that these and other substances known in the art of pharmacology can be included in a pharmaceutical composition.

In some embodiments, a nucleic acid, expression vector, or virion described herein is formulated with one or more biocompatible polymers. In some embodiments, a nucleic acid, expression vector, or virion described herein is formulated in a liposome. See, e.g., US 2017/0119666. In some embodiments, a nucleic acid, expression vector, or virion described herein is formulated in a nanoparticle. Nanoparticles include, e.g., polyalkylcyanoacrylate nanoparticles, nanoparticles comprising poly(lactic acid), nanoparticles comprising poly(lactic-co-glycolic acid) (PLGA) nanoparticles, and the like. In some embodiments, a nucleic acid, expression vector, or virion described herein is formulated in a hydrogel. Suitable hydrogel components include, but are not limited to, silk (see, e.g., U.S. Patent Publication No. 2017/0173161), poly(lactic acid) (PLA), poly(glycolic acid) (PGA), poly(lactide-co-glycolide) (PLGA), polyesters, hyaluronic acid, and the like. In some embodiments, a nucleic acid, expression vector, or virion described herein is present in a buffered saline solution. In some embodiments, the buffered saline solution between about 50 μL to 1000 μL in volume, including any range in between these values. In some embodiments, the 50 μL to 1000 μL in volume contains a unit dose of a nucleic acid, expression vector, or virion described herein.

In some embodiments, the unit dose of rAAV particles is in a pharmaceutical formulation. In some embodiments, the pharmaceutical formulation comprises the rAAV particles, one or more osmotic or ionic strength agents, one or more buffering agents, one or more surfactants, and one or more solvents. In some embodiments, the osmotic or ionic strength agent is sodium chloride. In some embodiments, the one or more buffering agents are sodium phosphate monobasic and/or sodium phosphate dibasic. In some embodiments, the surfactant is Poloxamer 188. In some embodiments, the solvent is water. In some embodiments, the pharmaceutical formulation comprises the rAAV particles, sodium chloride, sodium phosphate monobasic, sodium phosphate dibasic, and a surfactant.

### 5. Methods of Restoring or Enhancing Visual Function

In some embodiments, provided is a method of restoring or enhancing visual function in a subject, comprising administering a nucleic acid described herein, an expression vector described herein, a virion described herein, or a pharmaceutical composition described herein to the eye of a subject. In some embodiments, the subject is a mammal, e.g., a mouse, a human, or a non-human primate (e.g., a macaque or cynomolgus monkey). In some embodiments, the nucleic acid, expression vector, virion, or pharmaceutical composition described herein is administered via intravitreal (IVT) injection, subretinal (SR) injection, intraocular injection, or suprachoroidal injection. Other suitable modes of administration include, e.g., periocular injection, subconjunctive injection, retrobulbar injection, injection into the sclera, and intercameral injection. In some embodiments, the nucleic acid, expression vector, virion, or pharmaceutical composition described herein is administered to the subject as a single dose. In some embodiments, the nucleic acid, expression vector, virion, or pharmaceutical composition described herein is administered to the subject as a single injection.

In some embodiments, the nucleic acid, expression vector, virion, or pharmaceutical composition described herein is administered to the subject over a period of time ranging between about 1 day to about 1 year, including any range between these values (e.g., between about one week to about two weeks, between about two weeks and about 1 month, between about one month to and about 6 months, between about 6 months to about 1 year). In some embodiments, the nucleic acid, expression vector, virion, or pharmaceutical composition described herein is administered to the subject over a period of time that is greater than one year.

Following administration of the nucleic acid, expression vector, virion, or pharmaceutical composition, a CNGB3 described herein is produced in the retinal cell (e.g., a retinal ganglion cell, an amacrine cell, a horizontal cell, a bipolar cell, or a photoreceptor cell, such as a rod cell or a cone cell, Müller cell, or retinal pigmented epithelium cell), and expression of the CNGB3 protein in the retinal cell provides for enhanced or restored visual function in the subject. Tests for visual function are known in the art, and any known test can be applied to assess visual function in a subject administered with a nucleic acid, expression vector, virion, or pharmaceutical composition described herein.

In some embodiments, the subject is a human subject. In some embodiments, the human subject has a disease or a disorder that affects the retina. In some embodiments, the human subject has reduced visual function due to loss of functional cone photoreceptors. In some embodiments, the subject has an inherited retinal degenerative disease (IRD), such as achromatopsia. In some embodiments, the human subject has an ocular disease. In some embodiments, the human subject has an ocular disease, including, but not limited to, e.g., cone dystrophy.

### 6. Kits and Articles of Manufacture

In some embodiments, provided are kits or articles of manufacture that comprise one or more nucleic acids, expression vectors, virions, or pharmaceutical compositions disclosed herein for use according to a method or restoring or enhancing visual function described herein. In some embodiments, the kit comprises a lyophilized form of a pharmaceutical composition and a solution for reconstituting the pharmaceutical composition prior to administration to a subject. In some embodiments, the kit further comprises instructions for administering the one or more nucleic acids, expression vectors, virions, or pharmaceutical compositions herein to the eye of a subject (e.g., a human subject) via intravitreal injection, subretinal injection, intraocular injection, suprachoroidal injection, or other route of administration described herein.

In some embodiments, the kit comprises pharmaceutically acceptable excipients, buffers, solutions, etc. for administering the pharmaceutical composition. In some embodiments, the kit further comprises instructions for suitable operational parameters in the form of a label or a separate insert. For example, the kit may have standard instructions informing a physician or laboratory technician to prepare a therapeutically effective dose of the nucleic acid, expression vector, virion, or pharmaceutical composition and/or to reconstitute lyophilized compositions. In some embodiments, the kit further comprises a device for administration, such as a syringe, filter needle, extension tubing, cannula, or other implements to facilitate injection of the pharmaceutical composition to the eye of a subject. Exemplary injection routes are described elsewhere herein. In some embodiments, the kit comprises a pharmaceutical composition in the form of a suspension or refrigerated suspension, and a syringe and/or a buffer for dilution. In some embodiments, the kit comprises a pre-filled syringe comprising the suspension or refrigerated suspension.

The preceding description is presented to enable a person of ordinary skill in the art to make and use the various embodiments. Descriptions of specific compositions, techniques, and applications are provided only as examples. Various modifications to the embodiments described above will be readily apparent to those of ordinary skill in the art, and the general principles defined herein may be applied to other examples and applications without departing from the spirit and scope of the various embodiments. Thus, the various embodiments are not intended to be limited to the examples described herein and shown but are to be accorded the scope consistent with the claims.

### Materials and Methods

In Vitro Transfection of HEK293 with Plasmid Vectors Encoding Cone Photoreceptor Specific Promoter, to Assess Functionality and Protein Level.

The cone photoreceptor specific promoter MNTC (U.S. Pat. No. 10,000,741B2) and PR1.7 (patent #WO2014186160A1) are derived from the OPSIN-LCR human genomic locus. In addition, the MNTC promoter encodes for the human gene OPN1LW core promoter and a chimeric intron. These two promoters drive substantial level of transcription and lead to high transgene of expression in cone photoreceptors cells, but very low level in other cell types. Due to this very narrow cell type specificity, the expression of a therapeutic vector can be targeted exclusively to cone photoreceptors, without risk of ectopic expression which could be potentially harmful depending on the transgene. While these promoters' expression profile is very advantageous for AAV-mediated gene therapy addressing cone photoreceptor disfunction, this prevents any meaningful expression in commonly available cell lines, such as HEK293 or HELA. Here, we have used the dead CAS9 (dCAS9) CRISPRa synergistic activation mediator (SAM) system to circumvent this difficulty (Konermann et al. 2015 Nature, DOI: 10.1038/nature14136).

Briefly, the dCAS9 CRISPRa SAM corresponds to a programmable artificial transcription factor system, derived from clustered regularly interspaced short palindromic repeats (CRISPR) and nuclease deficient CRISPR associated protein 9 (dCas9). The dCAS9 is fused to the VP64 transcription effector factor domain. The guide RNA (gRNA) sequence defines the homing specificity and the target gene to be transactivated. Here, the gRNA is fused to a dimerized MS2 bacteriophage coat protein binding hairpin aptamer to form the gRNA scaffold. This scaffold domain can then recruit the MS2-p65-HSF1 fusion protein, composed of the MS2 bacteriophage coat protein, the NF-kappa-B (P65) and heat shock transcription factors. When targeted to the proximal region of a promoter, the CRISPRa SAM system has been shown to increase expression by up to 3-log over endogenous level (Chavez et al 2016 Nature Methods, DOI: 10.1038/NMETH.3871).

Here, we designed 7 different gRNA targeting either MNTC and/or PR1.7, to evaluate and compare the expression (identity, level, and molecular weight) from the different iteration of therapeutic expression cassettes, encoding CNGB3.

Briefly, the CRISPRa SAM system was purchased from Origene (cat #GE100057 Rockville MD). Three gRNA targeting the MNTC promoter were designed, synthetized and cloned at Origene i.e., GE200931A (GACCCTCAGGTGACGCACCA; SEQ ID NO:30), GE200931B (TCGACAAGCCCAGTTTCTAT; SEQ ID NO:31) and GE200931C (CGTTTCTGATAGGCACCTAT; SEQ ID NO:32). Four additional gRNA targeting both the MNTC and PR1.7 promoter were designed using https://chopchop.cbu.uib.no/and http://sam.genome-engineering.org/MNTC_ADVM_1 (GATCGGAGGAGGAGGTCTAAGTCCCG; SEQ ID NO:33), MNTC_ADVM_2 (GATCGGGAGCAGGGGAGCAAGAGGTG; SEQ ID NO:34), MNTC_ADVM_3 (GATCGCATTACCGCCATGAGCTGGCG; SEQ ID NO:35), MNTC_ADVM_4 (GATCGTATAAAGCACCGTGACCCTCG; SEQ ID NO:36).

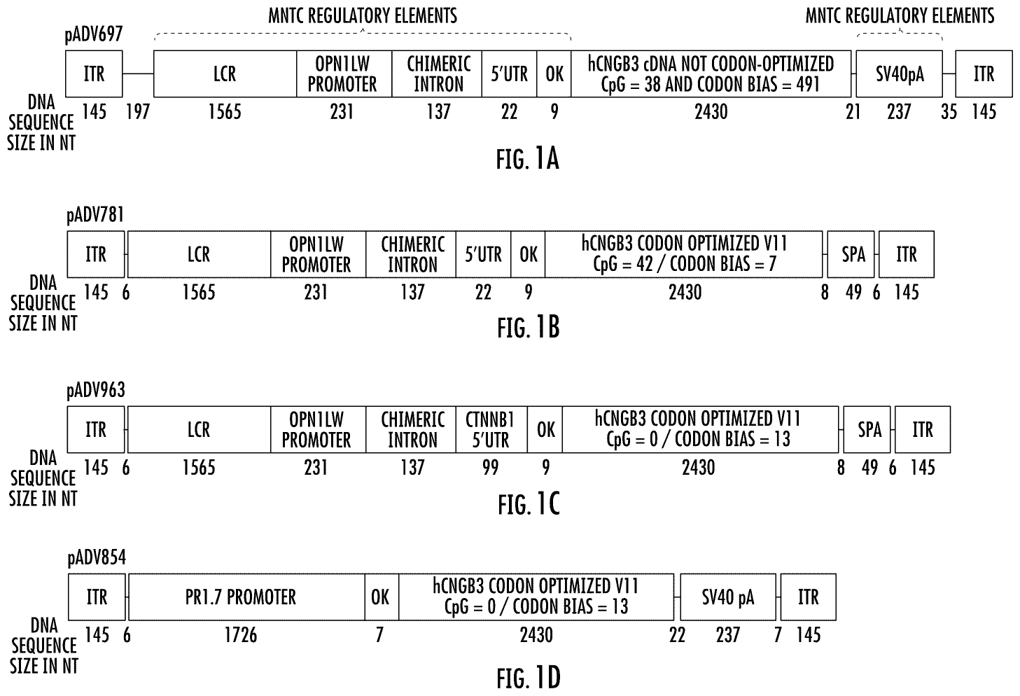

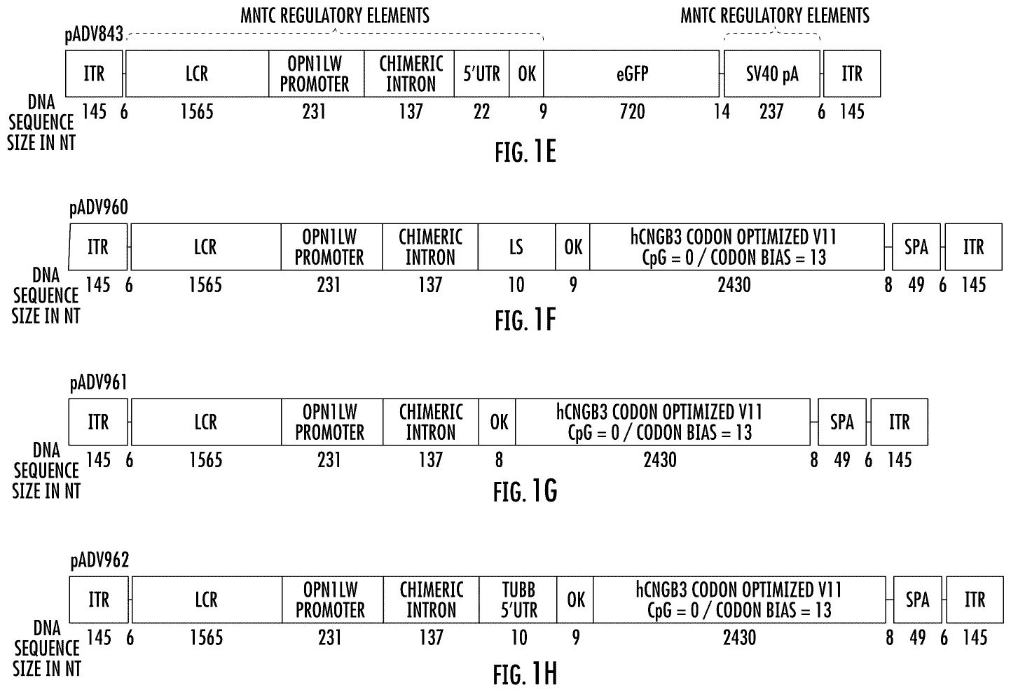

The gRNA MNTC_ADVM_1 was identified to drive the highest level of eGFP following transfection in HEK293 cell, following transient transfection with the plasmid pADV843-MNTC-eGFP-SV40 pA described in FIG. 1E, as well as following transduction with AAV2.7m8-MNTC-eGFP-SV40 pA derived from latter.

To assess protein expression level from CNGB3-encoding plasmid vector, the following protocol was performed. HEK293 were seeded in P6-wells at 1E6 cells per well, in complete cell culture media (IMDM, 10% SVF, 1% antibiotic). The following day, the complete media was replaced with Opti-MEM™ (ThermoFisher Scientific, cat #11058021) and 2 hours later, the cells were transfected. The transfection was performed with the following transfection mix for each P6 well: 8.75 μL of Lipofectamine™ 2000 (ThermoFisher Scientific, cat #11668019), 1.5 μg of transgene plasmid encoding CNGB3 or eGFP control, 1 μg of plasmid encoding dCAS9-VP64 and gRNA-scaffold and 1 μg of plasmid encoding the MS2-p65-HSF1 fusion protein. 24 hours later, the transfection and transactivation efficiency was evaluated with the GFP signal resulting from the pADV843 transfection control. At 48 hours post-transfection, the cells were harvested for protein analysis.

### Western Blot Analysis of CNGB3 Protein Level, Following In-Vitro Transient Transfection.

At approximately 48 hours post-transfection, cells were harvested following two rounds of cold PBS1× washes then centrifuged for 5 minutes at 1,000 RPM and 4° C., each round. The cells pellets were resuspended in RIPA Buffer (ThermoFisher Scientific, cat #89900) with 0.2% TritronX-100 (ThermoFisher Scientific, cat #85111), 0.2% IGEPAL® CA-630 (Sigma Aldrich, sku #13021), 0.2% SDS (Sigma Aldrich, sku #05030), complete™ Protease Inhibitor Cocktail EDTA free (Roche Diagnostics, 11873580001), and Roche PhosSTOP™ (Sigma Aldrich, sku #4906845001). The samples were then transferred into CK14 Precellys tubes (Bertin Corp, ref #P000912-LYSK0-A) and then lysed with the Precellys®24 evolution homogenizer (Bertin Corp) at 6000 RPM, 3×30 s and 4° C. Following centrifugation for 14,000 RPM for 40 minutes at 4° C., the supernatant was collected into low protein biding tubes (Eppendorf, cat #0030108442) and the protein concentration measured using the Pierce™ BCA Protein Assay Kit (ThermoFisher Scientific, cat #23227) and the SpectraMax M3 Plate Reader (Molecular Devices), accordingly to manufacturer instructions.

For Western Blot analysis, 7 μg of total protein extract were loaded and migrated in 4-12% Tris glycine SDS PAGE gels (ThermoFisher Scientific, Novex 4-12% Tris-glycine midi gels 10 wells, cat #XP04120BOX). Following electrophoretic migration for 45 minutes at 200V and room temperature, the proteins were transferred from the gel onto PVDF membranes (ThermoFisher Scientific, iBlot™ Transfer Stack, cat #IB401001), using the IBlot 2 Dry Blotting System and program P3 (ThermoFisher Scientific, cat #IB21001). To confirm homogenous and efficient protein transfer, the PVDF membranes were first stained with Ponceau S staining solution (ThermoFisher Scientific, cat #A40000278) and then washed. Afterward, the membranes were incubated for approximately one hour with the blocking solution (PBS1× Tween 0.05% Milk 5%), then immunoblotted overnight at 4° C. with the primary rabbit polyclonal antibody against CNGB3 ( 1/1,000, cat #PA5-66068, ThermoFisher Scientific) and then incubation for 90 minutes at room temperature with the secondary goat antibody anti-rabbit IgG HRP-conjugated ( 1/10,000, cat #7074, Cell Signaling). The luminescent signal was revealed using the SuperSignal West Dura Extended Duration Substrate (ThermoSienctific, 34075) and imaged with the Amersham ImageQuant 800 (Cytiva). For signal normalization, the membranes were then washed, blocked for 1 hour at room temperature and then immunoblotted overnight at 4° C. with the primary mouse anti-GAPDH antibody ( 1/10,000, MAB374, Millipore) and subsequently for approximately one hour at room temperature with the secondary goat antibody anti-mouse IgG HRP-conjugated ( 1/10,000, cat #7076, Cell Signaling). The signal was revealed and imaged similarly.

### AAV Vector Constructs, Production, and Quality Control.

Recombinant adeno-associated virus vectors (AAV) were produced by the triple plasmid transfection method in HEK293 and purified by ultracentrifugation on Iodixanol gradient and formulated in the following buffer 180 mM NaCl, 5 mM NaH₂P0₄, 5 mM Na₂HPO₄, 0.001% Pluronic F-68 (pH=7.3; 300-400 mOsm/kg H2O). The three AAV vector produced for in-vivo evaluation are AAV2.7m8-pADV843-MNTC-eGFP-SV40 pA, AAV2.7m8-pADV963-MNTC-5UTR.hCTNNB1-OK-CNGB3coV11-SPA and AAV2yF-pADV854-PR1.7-CNGB3coV11-SV40 pA. The corresponding vector constructs are described in FIG. 1C-D. Their respective titer quantified by Taqman qPCR are 1.09×10¹³, 1.28×10¹³ and 1.54×10¹³ vector genomes per mL (vg/mL). The following quality control assays were performed to assess AAV vector identity, purity, and functionality: SDS PAGE silver stain and/or Spyro-Orange stain, Western-blot anti-VP protein, endotoxin assay, in vitro transduction expression assay with eGFP signal observed directly by microscope imaging and CNGB3 protein level by Western Blot assay as described above.

### Animal Procedures

The Cngb3 Knock-Out (KO) mouse line was generated on a C57BL/6 genetic background, by constitutive and homozygote deletion in the second exon of the Cngb3 gene (Deltagen Inc., San Mateo, CA). The resulting messenger RNA is missing 16 nt between the position 733 and 749, affecting the transmembrane domain and protein level. C57BL/6 wild-type (WT) mice were sourced from Charles River Laboratories (Wilmington, MA). Housing animal facility was controlled for temperature (18-23° C.) and humidity (40-65%), with a 12-hours light/dark cycle (7 foot-candles during the light cycle) and free access to water and a standard rodent chow (Laboratory Rodent Diet 5001, Newco distributors, Brentwood, MO). All animal procedures and experiments were approved by the local Institutional Animal Care and Use Committees (University of Oklahoma Health Sciences Center, Oklahoma City, OK, IACUC protocol #21-026-EI) and conformed to the guidelines on the care and use of animals adopted by the Society for Neuroscience (Washington, D.C.) and the Association for Research in Vision and Ophthalmology (Rockville, MD).

### Subretinal Injection

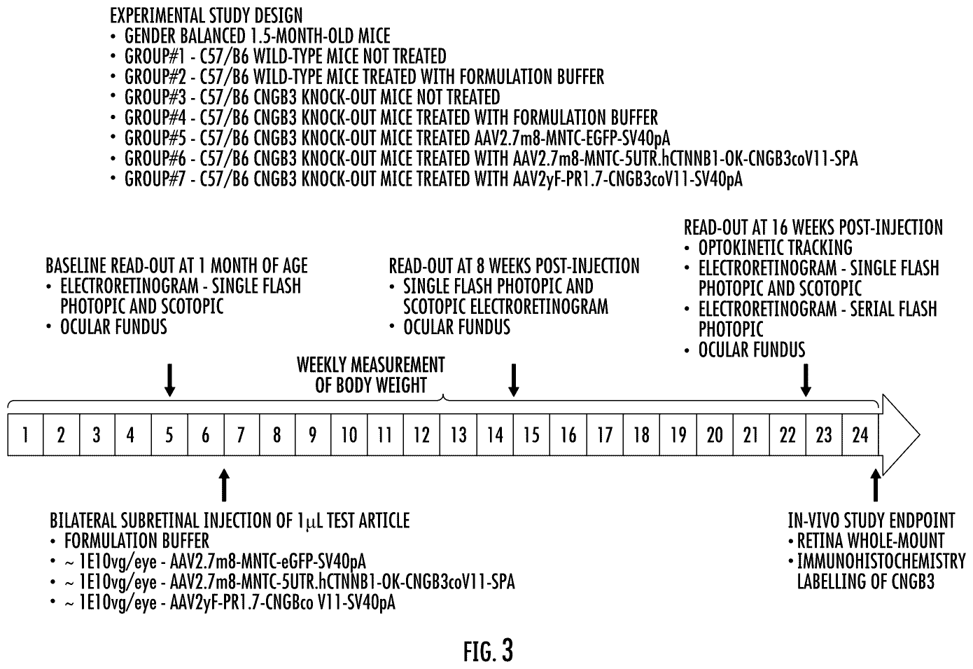

1.5-month-old animals were divided into seven different groups, according to the genotype, if treated or not and to test-article administrated, as described in FIG. 3. Mice were anesthetized by intraperitoneal injection of ketamine (85 mg/kg body weight) and xylazine (14 mg/kg body weight). The eyes were further anesthetized with topical 0.5% proparacaine drops and the pupils dilated with 1% tropicamide and 1% cyclopentolate drops (Akorn, Lake Forest, IL, USA). Bilateral injections of formulation buffer (1 μL volume) or one of AAV vectors (1 μL volume; 1×10¹⁰ vector genome) were performed under an OPMI VISU 140 surgical operating microscope (Zeiss, Thornwood, NY, USA). Subretinal injections were performed by way of a transscleral, transchoroidal approach, using the NanoFil microsyringe injector system with a 33-gauge blunt needle (Hamilton Co., Reno, NV, USA), with the animal in semi-prone position. The conjunctiva was cut close to the limbus and the sclera exposed. A shelving puncture of the sclera was made with a 30-gauge sharp needle. A blunt needle was then passed through this hole in a tangential direction. Following subretinal injection a circular bleb beneath the sclera was usually observed under the operating microscope. The needle was kept in the subretinal space for 1 minute, then withdrawn gently. The success of each subretinal injection was further confirmed by the observation of a partial retinal detachment by ocular fundoscopy. Post-operational care consisted in corneal application of antibiotic ointment (0.5% erythromycin) and Hypromellose ophthalmic demulcent (2.5%) to prevent infection and dehydration of the eye, as well as subcutaneous administration of meloxicam (4 mg/kg) to alleviate potential pain.

### Ocular Fundoscopy

Following anesthesia and pupil dilation, as described above, animals were placed in prone position on a heated-platform positioned, in front of the camera lens of the Phoenix the Phoenix MICRON™. The animals' head were held straight facing the narrow end of the platform and a sterile, viscous, glycol-based ophthalmic solution (2.5% Gonak, 1-2.5% Hypro methylcellulose) was applied directly to the corneal surface to lubricate the corneal epithelium. The stereoscopic focus was adjusted while slowly moving the camera toward the mouse until the lens contacts the ophthalmic gel on the cornea. The fundus was visualized through the display screen and the images captured.

### Electroretinogram Measurement

Full-field electroretinogram (ERG) recordings were carried out at baseline, 8- and 16-weeks post-dose. Animals were dark-adapted overnight and anesthetized during the procedure by intraperitoneal injection of ketamine-xylazine (85 mg/kg-14 mg/kg). ERGs were recorded using an Espion visual electrophysiology system with a Ganzfeld ColorDome system (Diagnosys LLC, MA, USA). Potentials were recorded using a gold-wire electrode to contact the corneal surface through a layer of 2.5% hypromellose (Gonak, Akorn). For assessment of scotopic responses, a stimulus intensity of 77 cd s m-2 was presented to dark-adapted dilated mouse eyes. To evaluate photopic responses, mice were adapted to a 25 cd s m-2 light for 5 minutes, and then exposed to a light intensity of 77 cd·s m-2 was given. Responses were differentially amplified, averaged, and analyzed using Espion 100 software (Diagnosys LLC). For serial photopic ERG recordings, animals were light-adapted to 25 cd s m-2 light for 5 minutes. In the Ganzfeld, mice were exposed to six series of 1-Hz light flashes, with increasing light intensities (0.1, 1, 3, 5, 10 and 20 cd s m-2), with each series separated by a 60 second light adaptation interval. Recordings measure consists in the average of 25 responses. Responses were differentially amplified, averaged, and stored according to the Espion V6 Software (Diagnosys LLC).

### Optokinetic Tracking (OKT)

Contrast sensitivities and visual acuities of treated and untreated eyes were measured by observing the optomotor responses of mice to rotating sinusoidal gratings (OptoMotry©), Cerebral Mechanics). Briefly, each mouse was placed on a pedestal located in the center of four inward facing LCD computer monitors screens and was observed by an overhead infrared video camera with infrared light source. Once the mouse became accustomed to the pedestal, a 7 s trial was initiated by presenting the mouse with the sinusoidal pattern rotating either clockwise or counter-clockwise as determined randomly by the OptoMotry© software. Visual acuity was measured under photopic conditions and defined as the highest spatial frequency (at 100% contrast) yielding a threshold response. Visual acuities were measured for each mouse at least three times on independent days by the same investigator.

### Euthanasia Procedures and Tissue Collection.

At 16-18 weeks post dosing and approximately 22-24 weeks of age, animals were euthanized by CO₂ inhalation asphyxiation, followed by cervical dislocation.

### Retina Whole-Mount Preparations and Histochemistry

Eyes were enucleated, marked at the superior pole with a green dye, and fixed in 4% PFA for 30-60 minutes at room temperature, followed by removal of the cornea and lens. The eyes were then fixed in 4% PFA in PBS for 4-6 hours at room temperature, and retinas were isolated, and the superior portion was marked for orientation with a small cut.

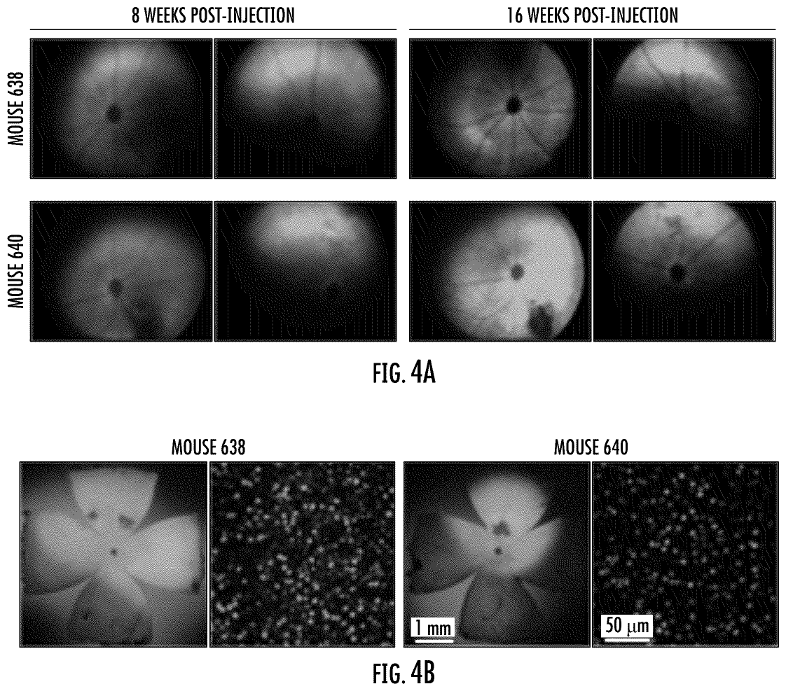

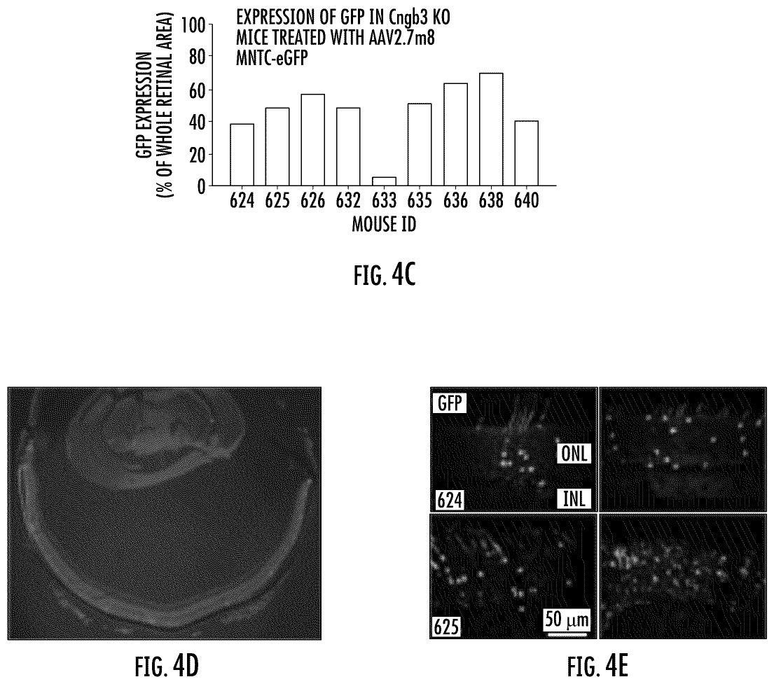

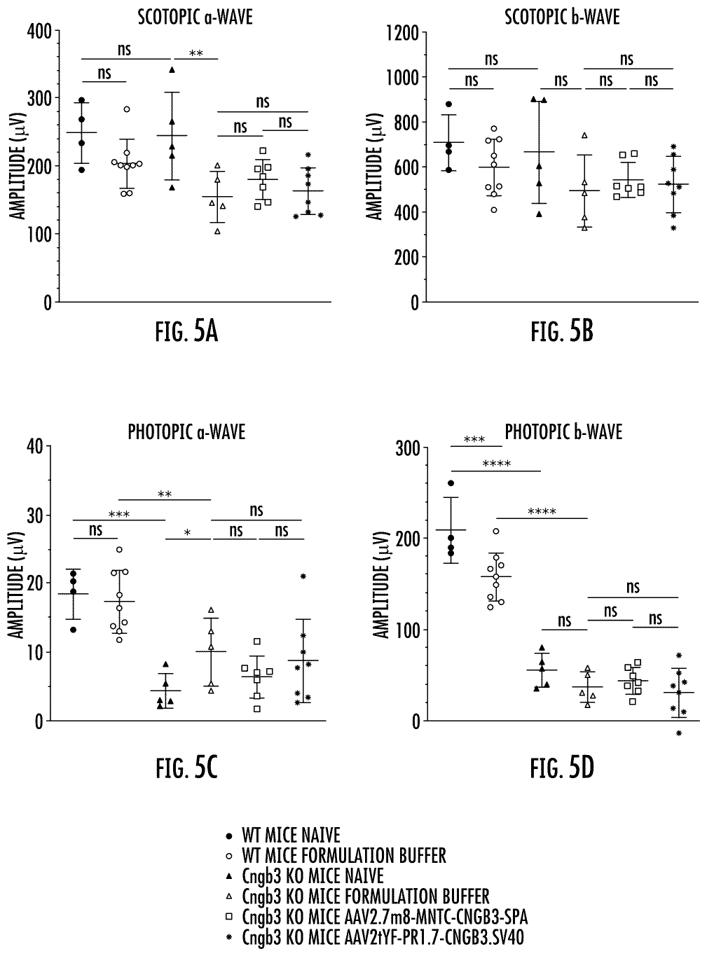

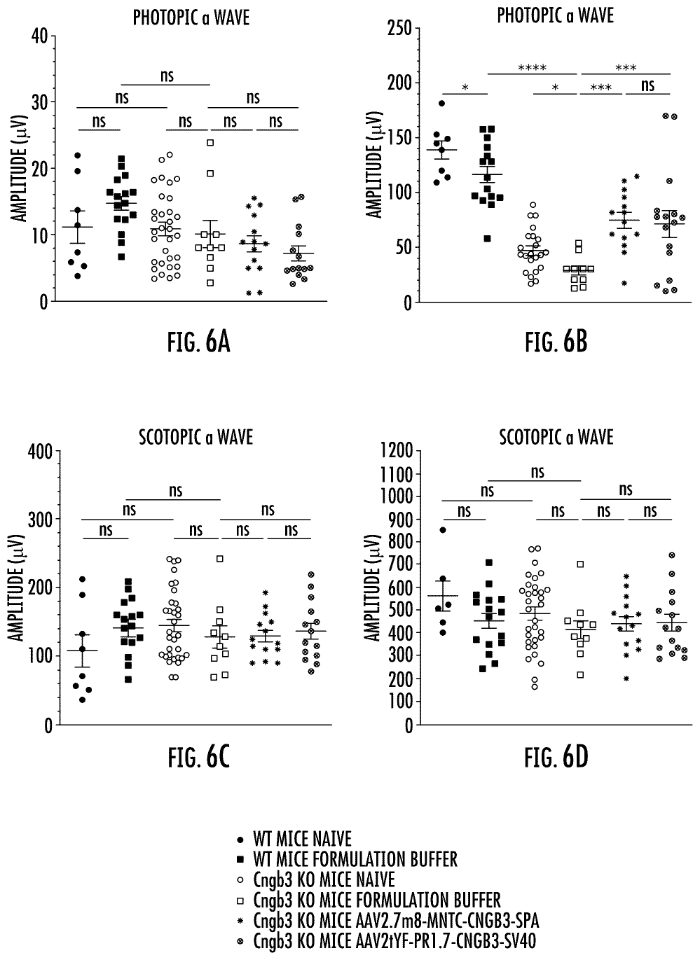

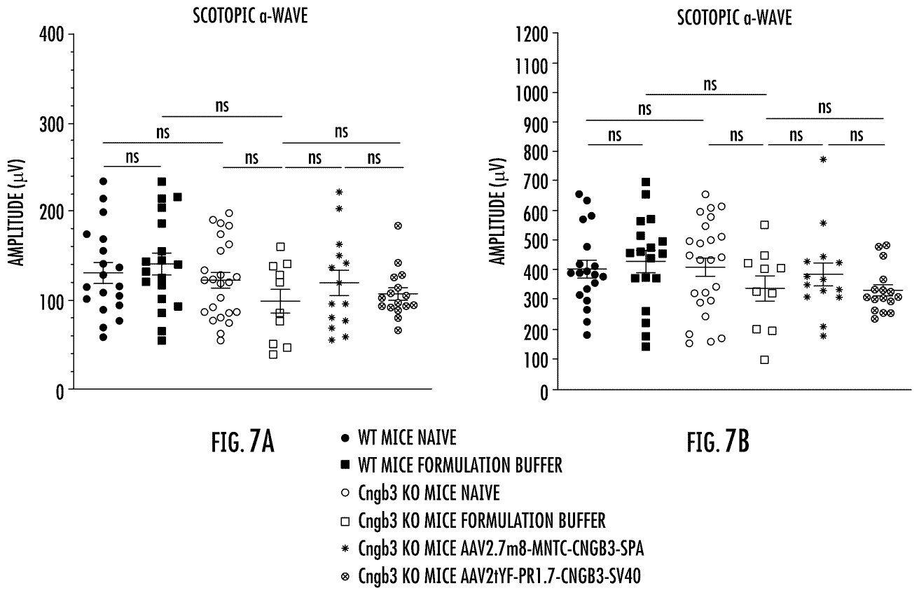

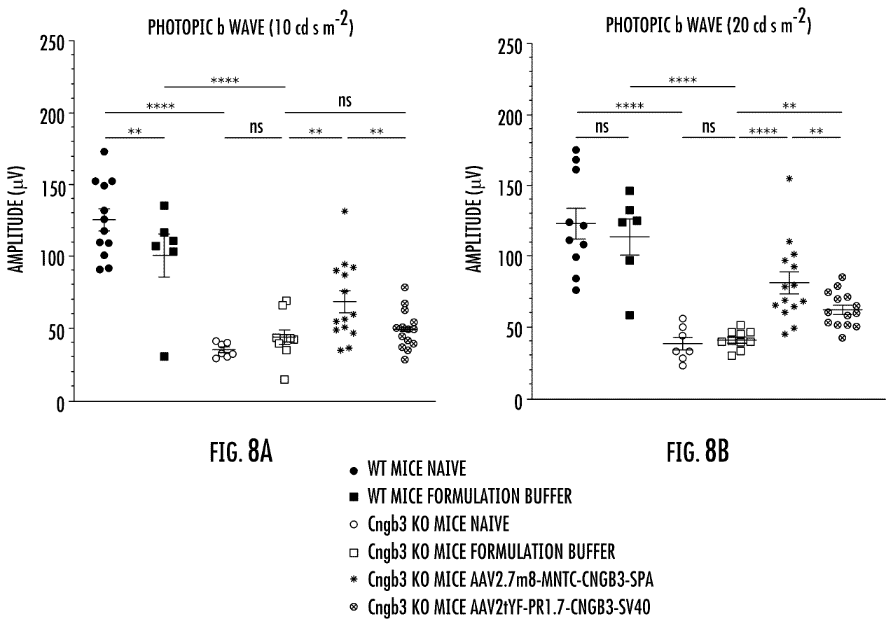

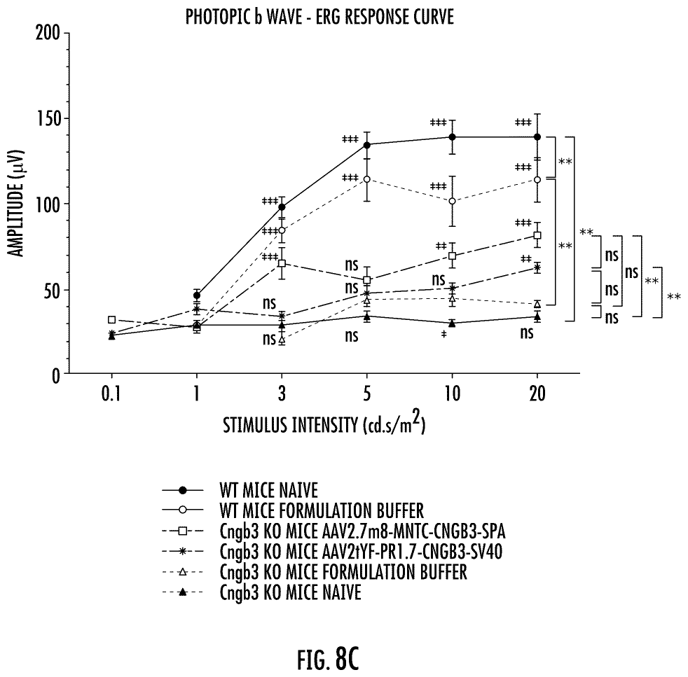

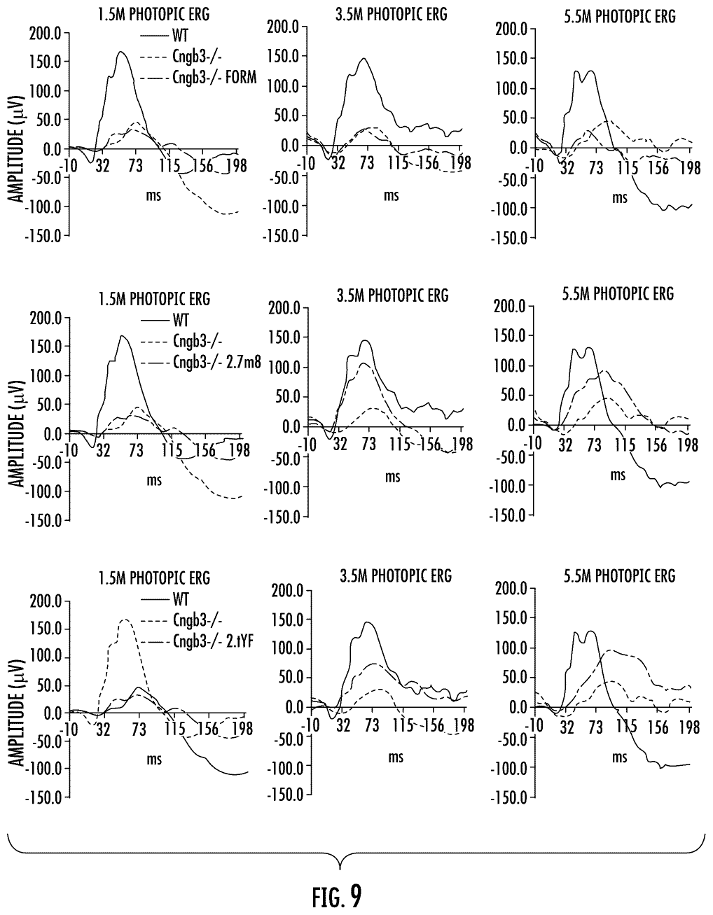

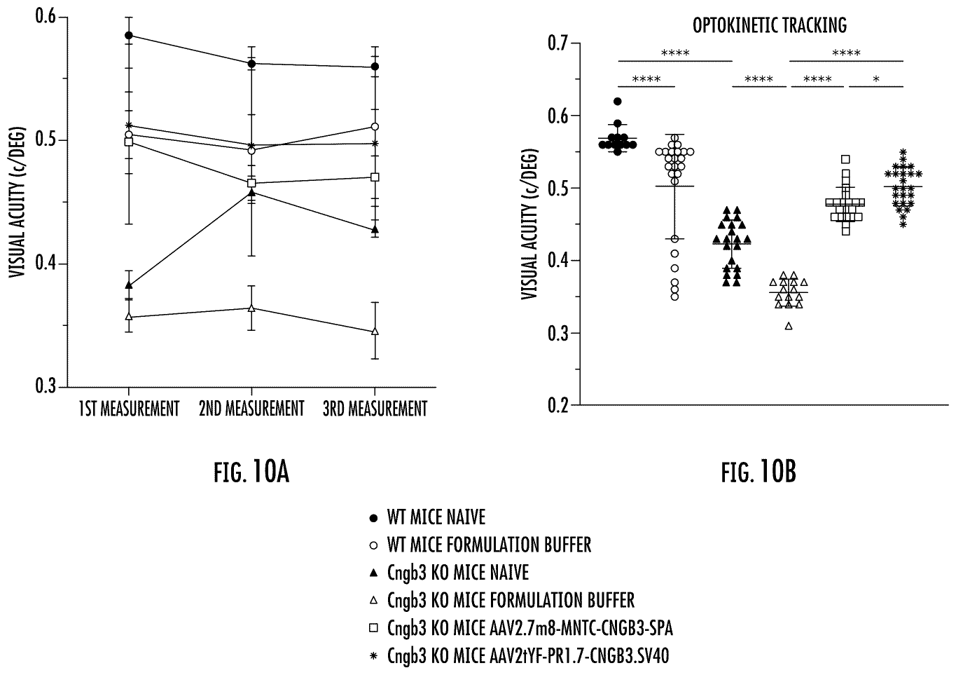

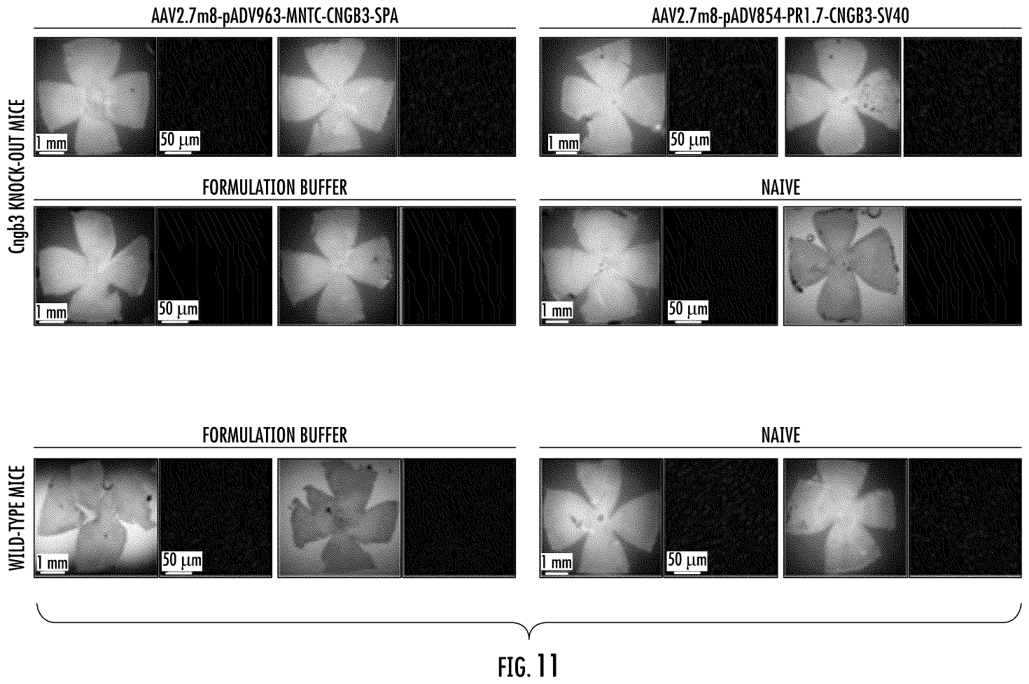

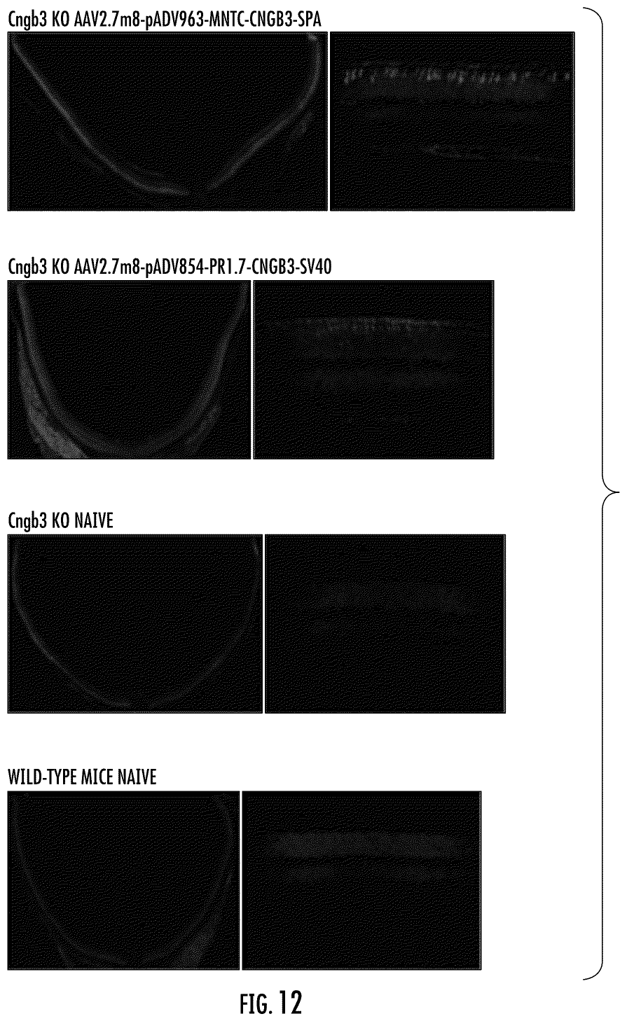

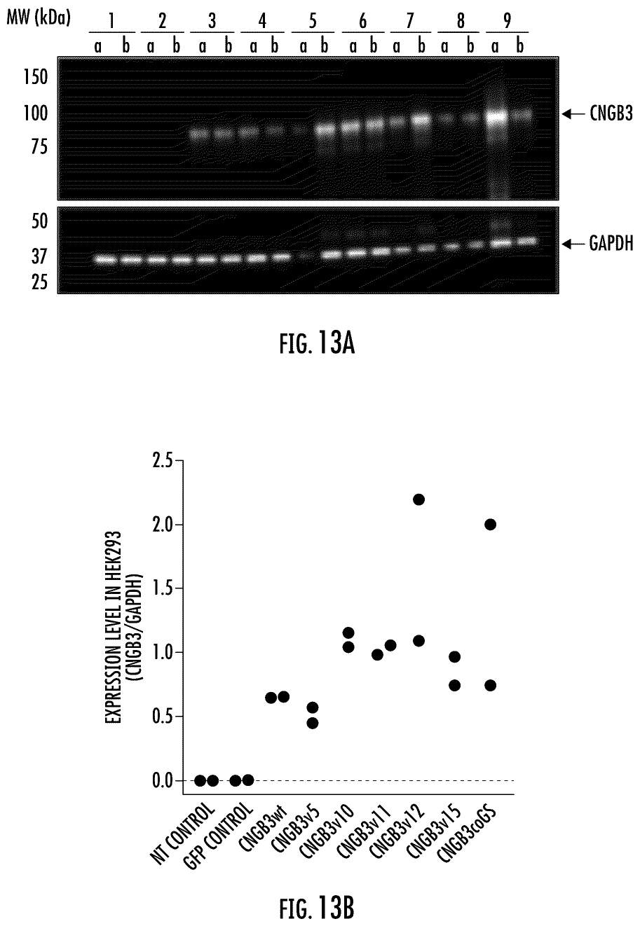

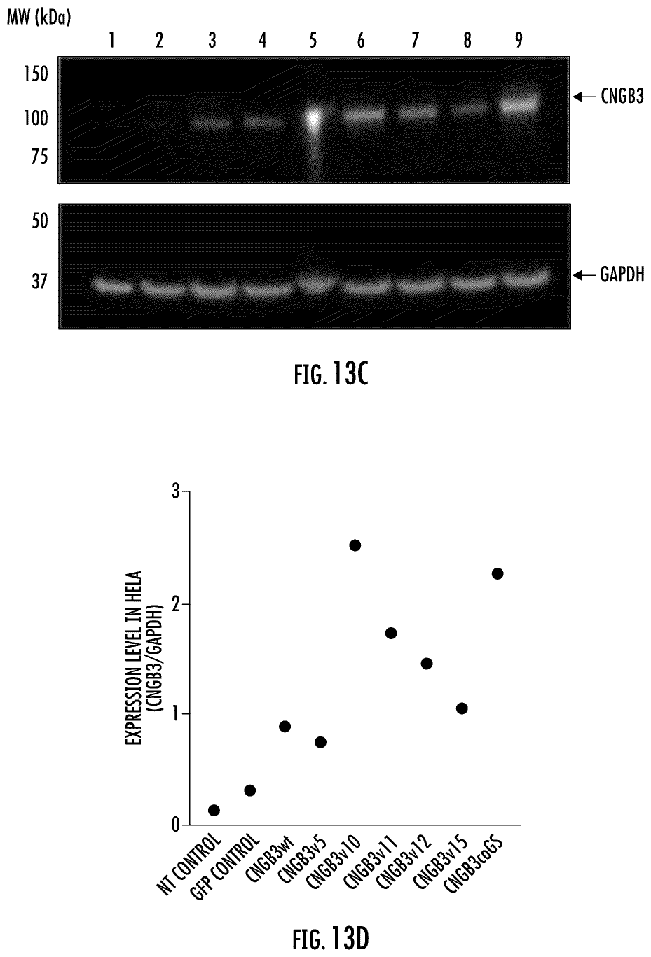

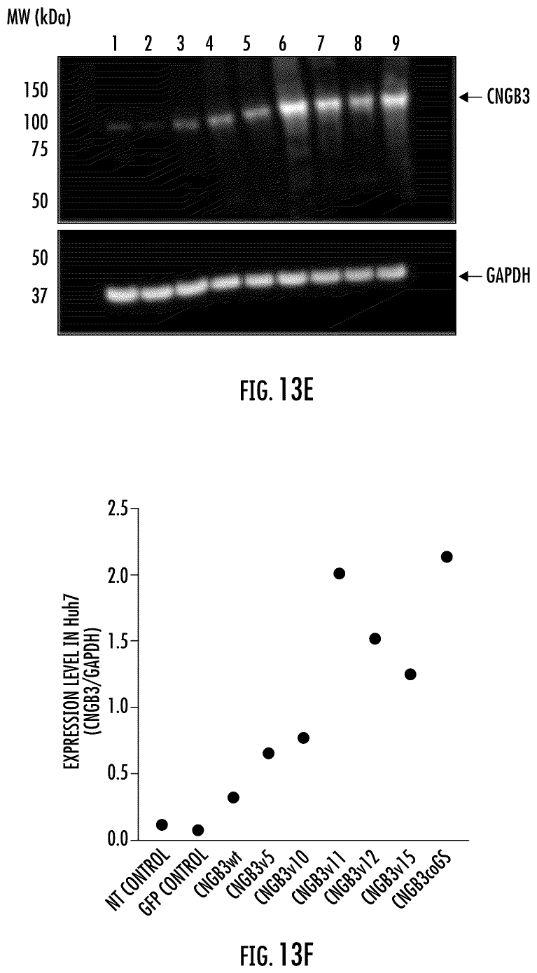

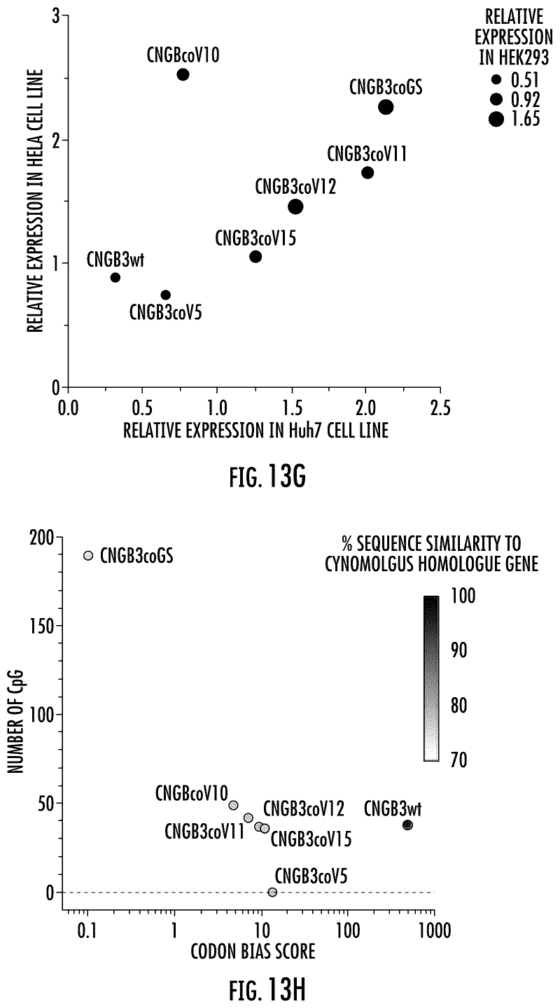

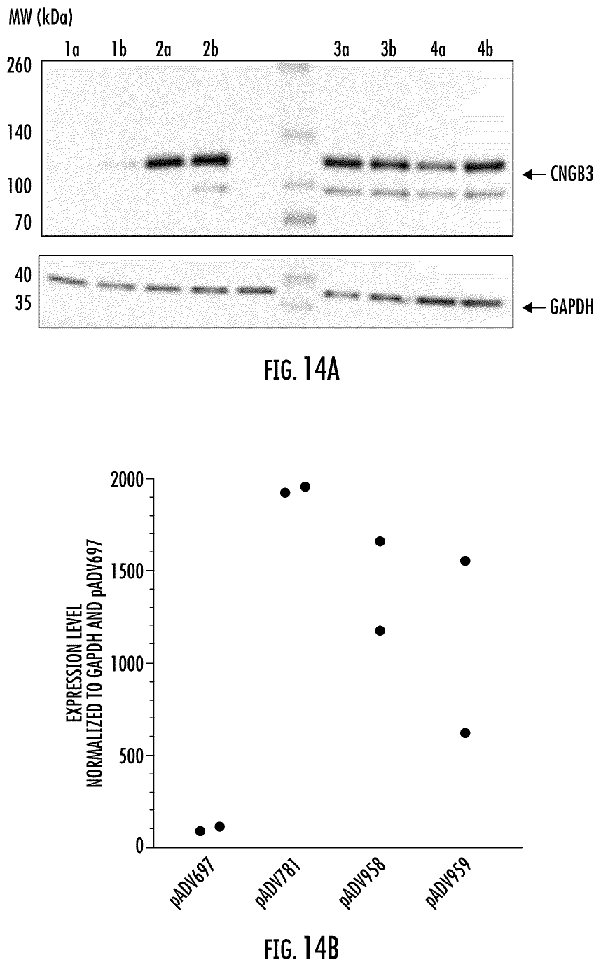

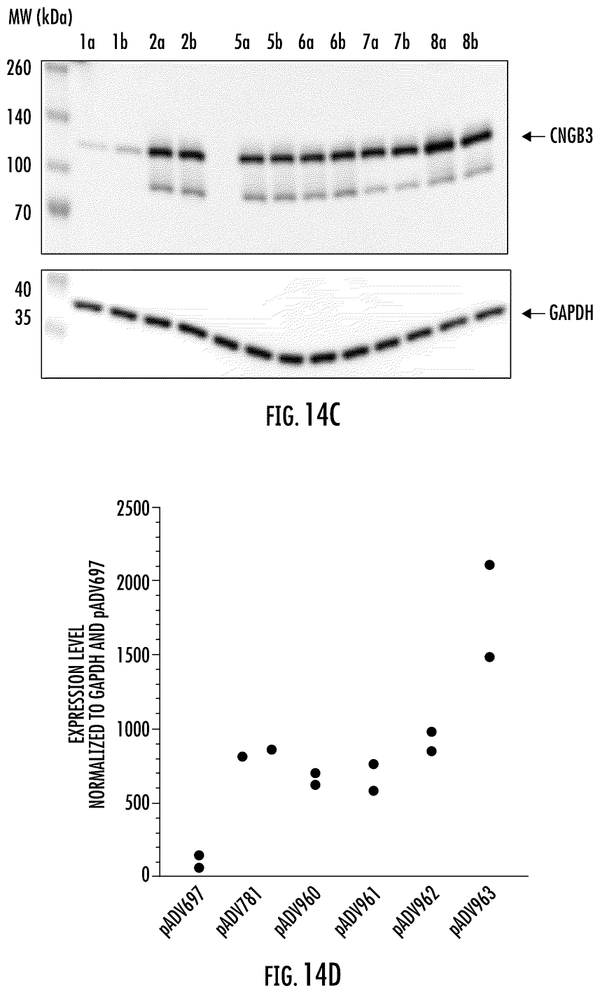

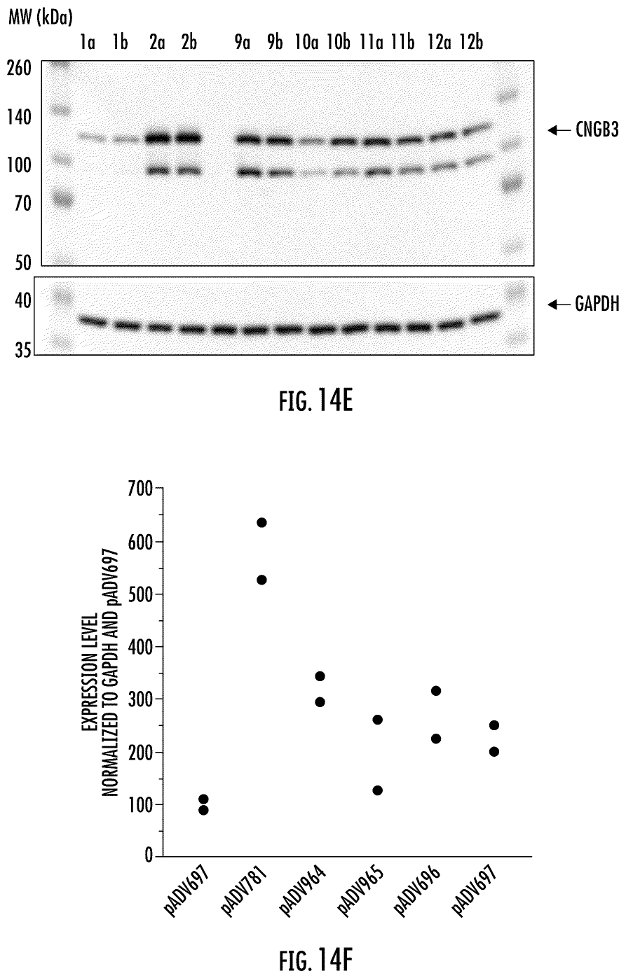

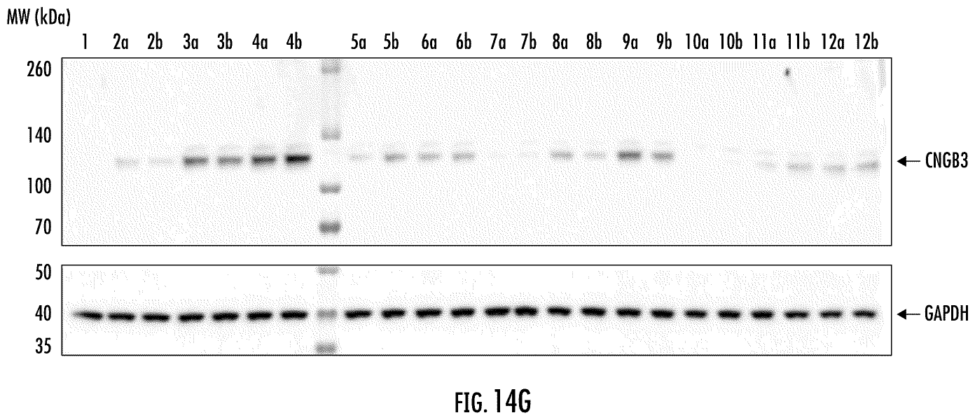

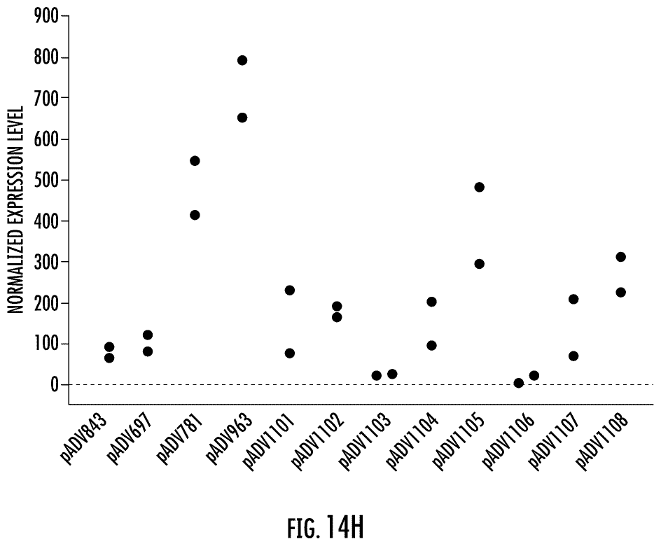

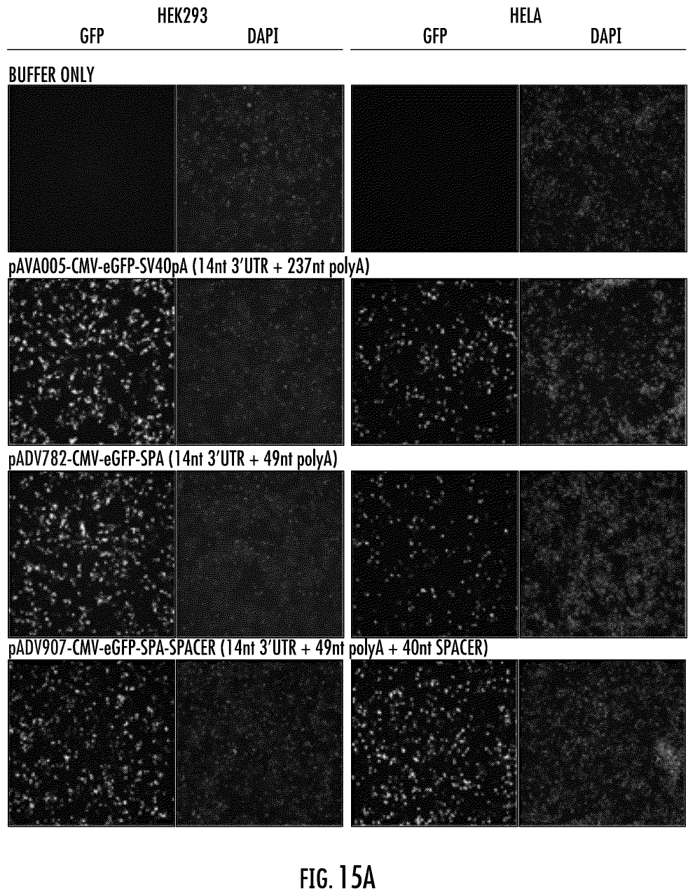

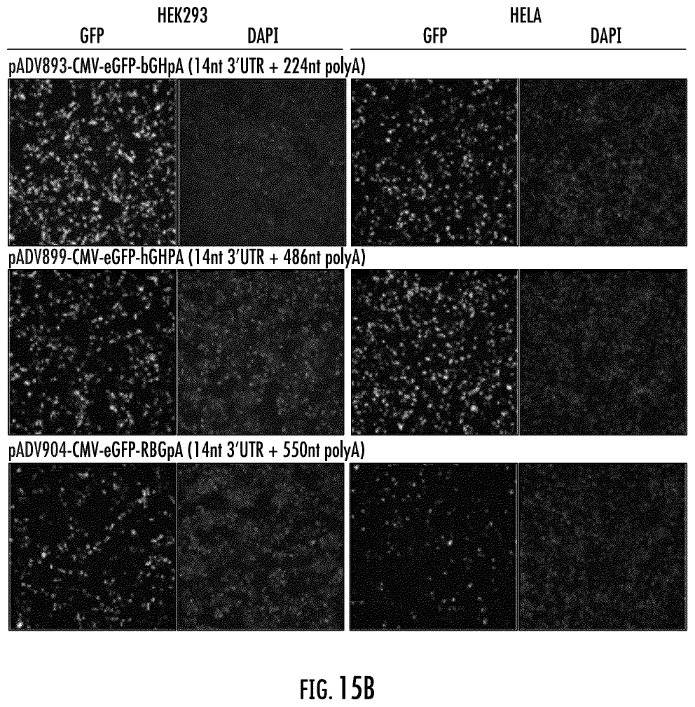

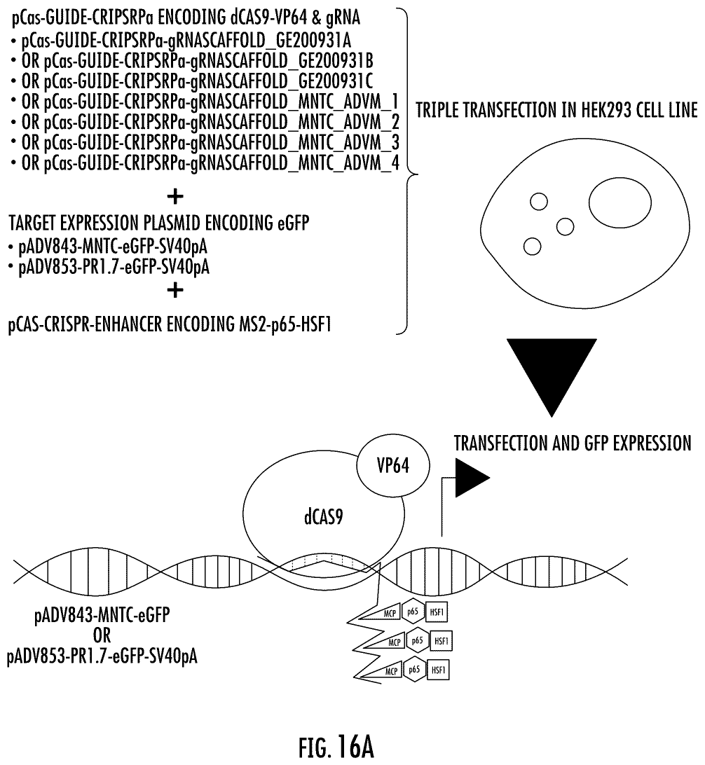

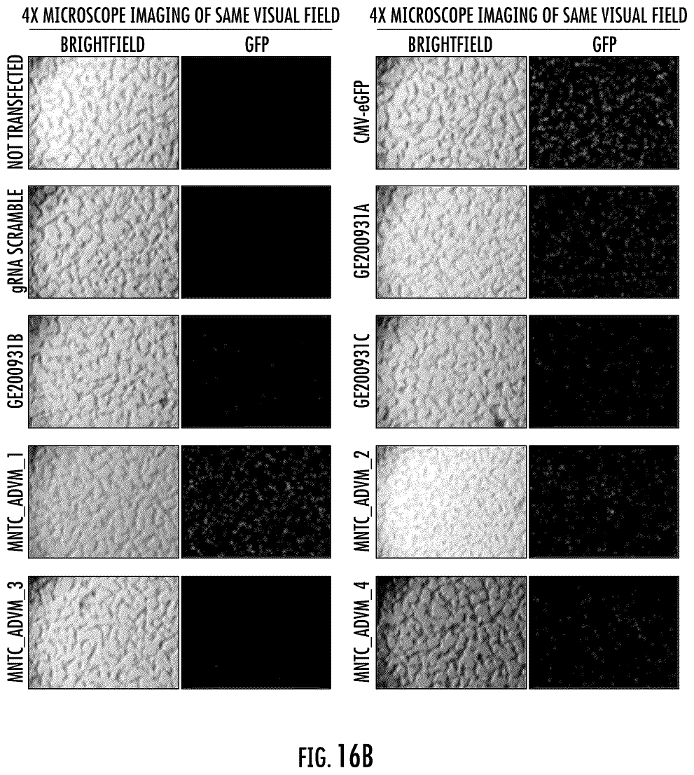

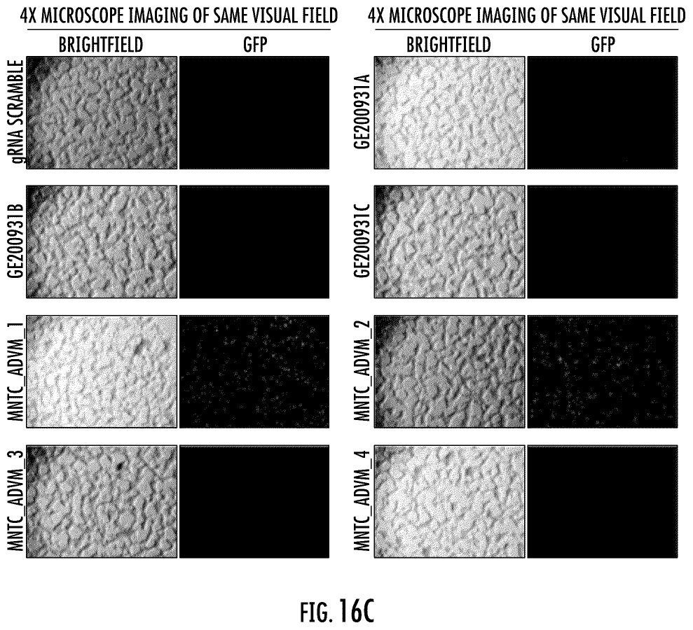

For mice treated with AAV2.7m8-MNTC-eGFP-SV40 pA and their respective controls, the native GFP signal was observed directly and imaged, as illustrated in FIG. 4B. For mice treated with AAV2.7m8-MNTC-5′UTRCtnnb1-CNGB3coV11-SPA or AAV2tYF-PR1.7-CNGB3coV11-SV40 pA and their respective controls, the CNGB3 protein was labelled by immunohistochemistry as illustrated in FIG. 11, with the CNGB3-C rabbit polyclonal antibody (dilution 1:100-200), developed in Dr Xi-Qin Ding's lab, which recognizes both the human and mouse CNGB3 proteins.

Briefly, retinal whole mounts were blocked in Hank's balanced salt solution containing 5% (wt/vol) BSA and 0.5% Triton-X 100 for 1 h at room temperature. Samples were incubated with primary antibody at room temperature for approximately two hours or at 4° C. overnight and then with AlexaFluor 568 nm-conjugated secondary antibody (dilution 1:500). Finally, retinal whole mounts were rinsed, mounted and imaged with the Olympus AX70 fluorescence microscope.

### Retina Paraffin Sections and Histochemistry

Following euthanasia, the superior portion of the cornea was marked with a green dye for orientation. Mouse eyes were enucleated, fixed with 4% formaldehyde (Polysciences, Inc., Warrington, PA) in 0.1 m sodium phosphate buffer, pH 7.4, for 16 h at 4° C. and then embedded in paraffin. 5-μm thick whole eye cross sections were prepared using a Leica microtome (Leica Biosystems, Buffalo Grove, IL)), transversal to the retina and along the vertical meridian passing through the optic-nerve head. For immunofluorescence labeling, eye sections were deparaffinized, rehydrated, and blocked with PBS containing 5% BSA and 0.5% Triton X-100 for 1 h at room temperature. Antigen retrieval treatment was performed by incubation in 10 mM sodium citrate buffer (pH 6.0) for 30 minutes in a water bath at 70° C. Primary antibody incubation was performed for approximately two hours at room temperature or 4° C. overnight. The rabbit polyclonal antibody PA5-6606 (dilution 1/50, ThermoFisher Scientific) was used to label specifically the human CNGB3 protein, without cross detection of the mouse endogenous CNGB3 protein. Following incubation with secondary antibody conjugated with AlexaFluor 568 nm and DAPI for 30 minutes at room temperature, tissue sections were mounted with glass coverslip. Fluorescent signals were imaged using an Olympus AX70 fluorescence microscope (Olympus Corp., Center Valley, PA) with QCapture imaging software (QImaging Corp., Surrey, BC, Canada) or an Olympus IX81-FV500 confocal laser scanning microscope (Olympus, Melville, NY) and FluoView imaging software (Olympus, Melville, NY).

### Statistical Analysis

Data were graphed and analyzed using Prism (GraphPad Software, CA, USA) as described in each figure legends.

Further advantages of the claimed subject matter will become apparent from the following examples describing certain embodiments of the claimed subject matter.
1. A polynucleotide cassette for enhanced expression of a transgene in cone cells of a mammalian retina, comprising a promoter region, wherein the promoter region is specific for retinal cone cells; an optimized 5′UTR sequence to increase translation of CNGB3 protein; a unique coding sequence optimized for high level of expression and low CpG, operatively linked to the promoter region wherein the coding sequence is a CNGB3 gene; and a polyadenylation site.
2. The polynucleotide cassette of Example 1 further comprising at least one AAV2 inverted terminal repeat (ITR).
3. The polynucleotide cassette of Example 1, further comprising two AAV2 ITR wherein one ITR is 5′ to the promoter and one ITR is 3′ to the polyadenylation site.
4. The polynucleotide cassette of Example 1, wherein the promoter is the SEQ ID NO: 14 or the SEQ ID NO: 15.
5. The polynucleotide cassette of Example 4, wherein the promoter further comprises a human opsin locus control region (LCR).
6. The polynucleotide cassette of Example 5, wherein the LCR is SEQ ID NO: 13.
7. The polynucleotide cassette of Example 1, wherein the optimized 5′ UTR is at least one of the SEQ ID NO: 18, SEQ ID NO: 19, SEQ ID NO:24 or SEQ ID NO:25.
8. The polynucleotide cassette of Example 1, wherein the unique coding sequence encodes the wild-type CNGB3 protein set forth in SEQ ID NO: 11 or a sequence having at least 75%, at least 80%, at least 85%, at least 90%, at least 95%, or at least 97% identity thereto.
9. The polynucleotide cassette of Example 1, wherein the unique coding sequence is SEQ ID NO: 20 or a sequence having at least 75%, at least 80%, at least 85%, at least 90%, at least 95%, or at least 97% identity thereto.
10. The polynucleotide cassette of Example 1, wherein the unique coding sequence is SEQ ID NO: 20.
11. The polynucleotide cassette of Example 1, wherein the polyadenylation site is SEQ ID NO: 22 or SEQ ID NO: 23.
12. The polynucleotide cassette of Example 1, further comprising at least one of the following elements: a human opsin locus control region (SEQ ID NO: 13); human beta catenin 1 5′UTR (CTNNB1 5′UTR) (SEQ ID NO:18); a 10 nt optimized lead sequence (SEQ ID NO:24); and Human beta tubulin gene (TUBB) 5′UTR (SEQ ID NO:25).
13. A polynucleotide cassette comprising in 5′ to 3′ orientation: optionally, a first ITR (SEQ ID NO: 12); optionally, human opsin locus control region (SEQ ID NO: 13); a promoter selected from SEQ ID NOs: 14 or 15; optionally, a chimeric intron (SEQ ID NO: 16); optionally, a 5′ UTR selected from SEQ ID NOs: 17, 18 or 25; optionally, a 10 nt optimized lead sequence (SEQ ID NO:24); an optimized Kozak sequence (SEQ ID NO: 19); a nucleotide sequence encoding a therapeutic protein; a polyA encoding nucleotide sequence selected from (SEQ ID NOs: 22 or 23); and optionally, a second ITR (SEQ ID NO: 12), wherein there is at least one ITR.
14. A polynucleotide cassette comprising SEQ ID NO: 1.
15. A polynucleotide cassette comprising SEQ ID NO: 2.
16. A polynucleotide cassette comprising SEQ ID NO: 3.
17. A polynucleotide cassette comprising SEQ ID NO: 4.
18. A polynucleotide cassette comprising SEQ ID NO: 5.
19. A polynucleotide cassette comprising SEQ ID NO: 26.
20. A polynucleotide cassette comprising SEQ ID NO: 27.
21. A polynucleotide cassette comprising SEQ ID NO: 28.
22. A recombinant virus comprising a variant capsid protein and the polynucleotide cassette of any of the preceding examples.
23. The recombinant virus of example 17 wherein the recombinant virus is a recombinant adeno-associated virus.
24. The recombinant virus of example 18, wherein the capsid protein is an AAV variant 7m8 capsid protein or is derived from the AAV variant 7m8 capsid protein.
25. A pharmaceutical composition comprising the recombinant virus of any one of examples 17-19 and a pharmaceutically acceptable excipient.
26. A method of treating achromotopsia in a human subject in need thereof, comprising administering to the subject a recombinant adeno-associated virus (rAAV) vector at a dosage ranging from about 1×10⁹ to about 1×10¹⁴ vector genomes (vg)/eye, wherein the rAAV vector comprises a CNGB3 gene, and wherein the rAAV vector comprises an AAV2 capsid variant that transduces foveal cone photoreceptors.
27. The method of Example 26, wherein the administration is selected from intravitreal (IVT) injection, subretinal (SR) injection, intraocular injection, or suprachoroidal injection.
28. The method of Example 27, wherein the administration is by intravitreal (IVT) injection.
29. The method of Example 27, wherein the administration is by intravitreal (IVT) injection.
30. The method of Example 26, wherein the AAV2 capsid variant comprises an AAV variant 7m8 capsid protein or is derived from the AAV variant 7m8 capsid protein.
31. The method of Example 30, wherein the AAV2 capsid variant comprises an AAV variant 7m8 capsid protein.
32. The method of Example 26, wherein the CNGB3 gene is a cDNA.
33. The method of Example 32, wherein the CNGB3 gene is codon optimized.
34. The method of Example 22, wherein the CNGB3 gene has lower CpG content than SEQ ID NO: 10.
35. The method of Example 32, wherein the CNGB3 gene is SEQ ID NO: 20.
36. A unit dosage form, comprising a recombinant adeno-associated virus (rAAV) vector at a dosage ranging from about 1×10⁹ to about 1×10¹⁴ vector genomes (vg)/eye, wherein the rAAV vector comprises a polynucleotide comprising a human CNGB3 protein coding sequence operably linked to a promoter sequence, and wherein the rAAV vector comprises an AAV2 capsid variant that transduces foveal cone photoreceptors.
37. The unit dosage form of Example 36 formulated for subretinal (SR) injection or intravitreal (IVT) injection.
38. The unit dosage form of Example 36 comprising 6×10⁹ to about 6×10¹¹ vector genomes.
39. An isolated host cell transfected or transduced with the polynucleotide cassette of example 1.

### SEQUENCE LISTING

| -pADV697-pFB-MNTC-CNGB3-SV40pA | | |
|---|---|---|
| SEQ ID NO: 1 | | |
| 1 | GCGCGCTCGC TCGCTCACTG AGGCCGCCCG GGCAAAGCCC GGGCGTCGGG | 50 |
| 51 | CGACCTTTGG TCGCCCGGCC TCAGTGAGCG AGCGAGCGCG CAGAGAGGGA | 100 |
| 101 | GTGGCCAACT CCATCACTAG GGGTTCCTTG TAGTTAATGA TTAACCCGCC | 150 |
| 151 | ATGCTACTTA TCTACGTAGC CATGCTCTAG GATCTTCAAT ATTGGCCATT | 200 |
| 201 | AGCCATATTA TTCATTGGTT ATATAGCATA AATCAATATT GGCTATTGGC | 250 |
| 251 | CATTGCATAC GTTGTATCTA TATCATAATA TGTACATTTA TATTGGCTCA | 300 |
| 301 | TGTCCAATAT GACCGCCATG TTGGCATTGA TTATTGACTA GTCCTACAGC | 350 |
| 351 | AGCCAGGGTG AGATTATGAG GCTGAGCTGA GAATATCAAG ACTGTACCGA | 400 |
| 401 | GTAGGGGGCC TTGGCAAGTG TGGAGAGCCC GGCAGCTGGG GCAGAGGGCG | 450 |
| 451 | GAGTACGGTG TGCGTTTACG GACCTCTTCA AACGAGGTAG GAAGGTCAGA | 500 |
| 501 | AGTCAAAAAG GGAACAAATG ATGTTTAACC ACACAAAAAT GAAAATCCAA | 550 |
| 551 | TGGTTGGATA TCCATTCCAA ATACACAAAG GCAACGGATA AGTGATCCGG | 600 |
| 601 | GCCAGGCACA GAAGGCCATG CACCCGTAGG ATTGCACTCA GAGCTCCCAA | 650 |
| 651 | ATGCATAGGA ATAGAAGGGT GGGTGCAGGA GGCTGAGGGG TGGGGAAAGG | 700 |
| 701 | GCATGGGTGT TTCATGAGGA CAGAGCTTCC GTTTCATGCA ATGAAAAGAG | 750 |
| 751 | TTTGGAGACG GATGGTGGTG ACTGGACTAT ACACTTACAC ACGGTAGCGA | 800 |
| 801 | TGGTACACTT TGTATTATGT ATATTTTACC ACGATCTTTT TAAAGTGTCA | 850 |
| 851 | AAGGCAAATG GCCAAATGGT TCCTTGTCCT ATAGCTGTAG CAGCCATCGG | 900 |
| 901 | CTGTTAGTGA CAAAGCCCCT GAGTCAAGAT GACAGCAGCC CCCATAACTC | 950 |
| 951 | CTAATCGGCT CTCCCGCGTG GAGTCATTTA GGAGTAGTCG CATTAGAGAC | 1000 |
| 1001 | AAGTCCAACA TCTAATCTTC CACCCTGGCC AGGGCCCCAG CTGGCAGCGA | 1050 |
| 1051 | GGGTGGGAGA CTCCGGGCAG AGCAGAGGGC GCTGACATTG GGGCCCGGCC | 1100 |
| 1101 | TGGCTTGGGT CCCTCTGGCC TTTCCCCAGG GGCCCTCTTT CCTTGGGGCT | 1150 |
| 1151 | TTCTTGGGCC GCCACTGCTC CCGCTCCTCT CCCCCCATCC CACCCCCTCA | 1200 |
| 1201 | CCCCCTCGTT CTTCATATCC TTCTCTAGTG CTCCCTCCAC TTTCATCCAC | 1250 |
| 1251 | CCTTCTGCAA GAGTGTGGGA CCACAAATGA GTTTTCACCT GGCCTGGGGA | 1300 |
| 1301 | CACACGTGCC CCCACAGGTG CTGAGTGACT TTCTAGGACA GTAATCTGCT | 1350 |
| 1351 | TTAGGCTAAA ATGGGACTTG ATCTTCTGTT AGCCCTAATC ATCAATTAGC | 1400 |
| 1401 | AGAGCCGGTG AAGGTGCAGA ACCTACCGCC TTTCCAGGCC TCCTCCCACC | 1450 |
| 1451 | TCTGCCACCT CCACTCTCCT TCCTGGGATG TGGGGGCTGG CACACGTGTG | 1500 |
| 1501 | GCCCAGGGCA TTGGTGGGAT TGCACTGAGC TGGGTCATTA GCGTAATCCT | 1550 |
| 1551 | GGACAAGGGC AGACAGGGCG AGCGGAGGGC CAGCTCCGGG GCTCAGGCAA | 1600 |
| 1601 | GGCTGGGGGC TTCCCCCAGA CACCCCACTC CTCCTCTGCT GGACCCCCAC | 1650 |
| 1651 | TTCATAGGGC ACTTCGTGTT CTCAAAGGGC TTCCAAATAG CATGGTGGCC | 1700 |
| 1701 | TTGGATGCCC AGGGAAGCCT CAGAGTTGCT TATCTCCCTC TAGACAGAAG | 1750 |
| 1751 | GGGAATCTCG GTCAAGAGGG AGAGGTCGCC CTGTTCAAGG CCACCCAGCC | 1800 |
| 1801 | AGCTCATGGC GGTAATGGGA CAAGGCTGGC CAGCCATCCC ACCCTCAGAA | 1850 |
| 1851 | GGGACCCGGT GGGGCAGGTG ATCTCAGAGG AGGCTCACTT CTGGGTCTCA | 1900 |
| 1901 | CATTCTTCCA GCAAATCCCT CTGAGCCGCC CCCGGGGGCT CGCCTCAGGA | 1950 |
| 1951 | GCAAGGAAGC AAGGGGTGGG AGGAGGAGGT CTAAGTCCCA GGCCCAATTA | 2000 |
| 2001 | AGAGATCAGA TGGTGTAGGA TTTGGGAGCT TTTAAGGTGA AGAGGCCCGG | 2050 |
| 2051 | GCTGATCCCA CTGGCCGGTA TAAAGCACCG TGACCCTCAG GTGACGCACC | 2100 |
| 2101 | AGGGCCGGCT GCCGTCGGGG ACAGGGCTTT CCATAGCCCA GGTAAGTATC | 2150 |
| 2151 | AAGGTTACAA GACAGGTTTA AGGAGACCAA TAGAAACTGG GCTTGTCGAG | 2200 |
| 2201 | ACAGAGAAGA CTCTTGCGTT TCTGATAGGC ACCTATTGGT CTTACTGACA | 2250 |
| 2251 | TCCACTTTGC CTTTCTCTCC ACAGGCCCAG AGAGGAGACA GGCTAGCGCC | 2300 |
| 2301 | GCCACCATGT TTAAATCGCT GACAAAAGTC AACAAGGTGA AGCCTATAGG | 2350 |
| 2351 | AGAGAACAAT GAGAATGAAC AAAGTTCTCG TCGGAATGAA GAAGGCTCTC | 2400 |
| 2401 | ACCCAAGTAA TCAGTCTCAG CAAACCACAG CACAGGAAGA AAACAAAGGT | 2450 |
| 2451 | GAAGAGAAAT CTCTCAAAAC CAAGTCAACT CCAGTCACGT CTGAAGAGCC | 2500 |
| 2501 | ACACACCAAC ATACAAGACA AACTCTCCAA GAAAAATTCC TCTGGAGATC | 2550 |
| 2551 | TGACCACAAA CCCTGACCCT CAAAATGCAG CAGAACCAAC TGGAACAGTG | 2600 |
| 2601 | CCAGAGCAGA AGGAAATGGA CCCCGGGAAA GAAGGTCCAA ACAGCCCACA | 2650 |
| 2651 | AAACAAACCG CCTGCAGCTC CTGTTATAAA TGAGTATGCC GATGCCCAGC | 2700 |
| 2701 | TACACAACCT GGTGAAAAGA ATGCGTCAAA GAACAGCCCT CTACAAGAAA | 2750 |
| 2751 | AAGTTGGTAG AGGGAGATCT CTCCTCACCC GAAGCCAGCC CACAAACTGC | 2800 |
| 2801 | AAAGCCCACG GCTGTACCAC CAGTAAAAGA AAGCGATGAT AAGCCAACAG | 2850 |
| 2851 | AACATTACTA CAGGCTGTTG TGGTTCAAAG TCAAAAAGAT GCCTTTAACA | 2900 |
| 2901 | GAGTACTTAA AGCGAATTAA ACTTCCAAAC AGCATAGATT CATACACAGA | 2950 |
| 2951 | TCGACTCTAT CTCCTGTGGC TCTTGCTTGT CACTCTTGCC TATAACTGGA | 3000 |
| 3001 | ACTGCTGTTT TATACCACTG CGCCTCGTCT TCCCATATCA AACCGCAGAC | 3050 |
| 3051 | AACATACACT ACTGGCTTAT TGCGGACATC ATATGTGATA TCATCTACCT | 3100 |
| 3101 | TTATGATATG CTATTTATCC AGCCCAGACT CCAGTTTGTA AGAGGAGGAG | 3150 |
| 3151 | ACATAATAGT GGATTCAAAT GAGCTAAGGA AACACTACAG GACTTCTACA | 3200 |
| 3201 | AAATTTCAGT TGGATGTCGC ATCAATAATA CCATTTGATA TTTGCTACCT | 3250 |
| 3251 | CTTCTTTGGG TTTAATCCAA TGTTTAGAGC AAATAGGATG TTAAAGTACA | 3300 |
| 3301 | CTTCATTTTT TGAATTTAAT CATCACCTAG AGTCTATAAT GGACAAAGCA | 3350 |
| 3351 | TATATCTACA GAGTTATTCG AACAACTGGA TACTTGCTGT TTATTCTGCA | 3400 |
| 3401 | CATTAATGCC TGTGTTTATT ACTGGGCTTC AAACTATGAA GGAATTGGCA | 3450 |
| 3451 | CTACTAGATG GGTGTATGAT GGGGAAGGAA ACGAGTATCT GAGATGTTAT | 3500 |
| 3501 | TATTGGGCAG TTCGAACTTT AATTACCATT GGTGGCCTTC CAGAACCACA | 3550 |
| 3551 | AACTTTATTT GAAATTGTTT TTCAACTCTT GAATTTTTTT TCTGGAGTTT | 3600 |
| 3601 | TTGTGTTCTC CAGTTTAATT GGTCAGATGA GAGATGTGAT TGGAGCAGCT | 3650 |
| 3651 | ACAGCCAATC AGAACTACTT CCGCGCCTGC ATGGATGACA CCATTGCCTA | 3700 |
| 3701 | CATGAACAAT TACTCCATTC CTAAACTTGT GCAAAAGCGA GTTCGGACTT | 3750 |
| 3751 | GGTATGAATA TACATGGGAC TCTCAAAGAA TGCTAGATGA GTCTGATTTG | 3800 |
| 3801 | CTTAAGACCC TACCAACTAC GGTCCAGTTA GCCCTCGCCA TTGATGTGAA | 3850 |
| 3851 | CTTCAGCATC ATCAGCAAAG TCGACTTGTT CAAGGGTTGT GATACACAGA | 3900 |
| 3901 | TGATTTATGA CATGTTGCTA AGATTGAAAT CCGTTCTCTA TTTGCCTGGT | 3950 |
| 3951 | GACTTTGTCT GCAAAAAGGG AGAAATTGGC AAGGAAATGT ATATCATCAA | 4000 |
| 4001 | GCATGGAGAA GTCCAAGTTC TTGGAGGCCC TGATGGTACT AAAGTTCTGG | 4050 |
| 4051 | TTACTCTGAA AGCTGGGTCG GTGTTTGGAG AAATCAGCCT TCTAGCAGCA | 4100 |
| 4101 | GGAGGAGGAA ACCGTCGAAC TGCCAATGTG GTGGCCCACG GGTTTGCCAA | 4150 |
| 4151 | TCTTTTAACT CTAGACAAAA AGACCCTCCA AGAAATTCTA GTGCATTATC | 4200 |
| 4201 | CAGATTCTGA AAGGATCCTC ATGAAGAAAG CCAGAGTGCT TTTAAAGCAG | 4250 |
| 4251 | AAGGCTAAGA CCGCAGAAGC AACCCCTCCA AGAAAAGATC TTGCCCTCCT | 4300 |
| 4301 | CTTCCCACCG AAAGAAGAGA CACCCAAACT GTTTAAAACT CTCCTAGGAG | 4350 |
| 4351 | GCACAGGAAA AGCAAGTCTT GCAAGACTAC TCAAATTGAA GCGAGAGCAA | 4400 |
| 4401 | GCAGCTCAGA AGAAAGAAAA TTCTGAAGGA GGAGAGGAAG AAGGAAAAGA | 4450 |
| 4451 | AAATGAAGAT AAACAAAAAG AAAATGAAGA TAAACAAAAA GAAAATGAAG | 4500 |
| 4501 | ATAAAGGAAA AGAAAATGAA GATAAAGATA AAGGAAGAGA GCCAGAAGAG | 4550 |
| 4551 | AAGCCACTGG ACAGACCTGA ATGTACAGCA AGTCCTATTG CAGTGGAGGA | 4600 |
| 4601 | AGAACCCCAC TCAGTTAGAA GGACAGTTTT ACCCAGAGGG ACTTCTCGTC | 4650 |
| 4651 | AATCACTCAT TATCAGCATG GCTCCTTCTG CTGAGGGCGG AGAAGAGGTT | 4700 |
| 4701 | CTTACTATTG AAGTCAAAGA AAAGGCTAAG CAATGAATCT AGAGCGGCCG | 4750 |
| 4751 | CTTCGAGCAG ACATGATAAG ATACATTGAT GAGTTTGGAC AAACCACAAC | 4800 |
| 4801 | TAGAATGCAG TGAAAAAAAT GCTTTATTTG TGAAATTTGT GATGCTATTG | 4850 |
| 4851 | CTTTATTTGT AACCATTATA AGCTGCAATA AACAAGTTAA CAACAACAAT | 4900 |
| 4901 | TGCATTCATT TTATGTTTCA GGTTCAGGGG GAGATGTGGG AGGTTTTTTA | 4950 |
| 4951 | AAGCAAGTAA AACCTCTACA AATGTGGTAA AATCGATAAG GATCCTAGAG | 5000 |
| 5001 | CATGGCTACG TAGATAAGTA GCATGGCGGG TTAATCATTA ACTACAAGGA | 5050 |
| 5051 | ACCCCTAGTG ATGGAGTTGG CCACTCCCTC TCTGCGCGCT CGCTCGCTCA | 5100 |
| 5101 | CTGAGGCCGG GCGACCAAAG GTCGCCCGAC GCCCGGGCTT TGCCCGGGCG | 5150 |
| 5151 | GCCTCAGTGA GCGAGCGAGC GCGC | 5174 |

| Name | Start | End |
|---|---|---|
| ITR, inverse terminal repeat from AAV2 | 1 | 145 |
| LCR, human opsin locus control region | 343 | 1907 |
| OPN1LW promoter, Human OPN1LW core promoter | 1908 | 2138 |
| Chimeric intron | 2139 | 2275 |
| 5′UTR | 2276 | 2291 |
| OK, optimized Kozak | 2298 | 2306 |
| hCNGB3 cDNA, native cDNA sequence encoding the | 2307 | 4736 |
| human cyclic nucleotide gated channel subunit beta 3 | | |
| (CNGB3wt) | | |
| SV40pA, Simian virus 40 polyadenylation sequence | 4758 | 4994 |
| ITR, inverse terminal repeat from AAV2 | 5030 | 5174 |

| -pADV781-MNTC-5UTR-OK-CNGB3coV11-SPA | | |
|---|---|---|
| SEQ ID NO: 2 | | |
| 1 | GCGCGCTCGC TCGCTCACTG AGGCCGCCCG GGCAAAGCCC GGGCGTCGGG | 50 |
| 51 | CGACCTTTGG TCGCCCGGCC TCAGTGAGCG AGCGAGCGCG CAGAGAGGGA | 100 |
| 101 | GTGGCCAACT CCATCACTAG GGGTTCCTTG TAGTTAATGA TTAACACTAG | 150 |
| 151 | TCCTACAGCA GCCAGGGTGA GATTATGAGG CTGAGCTGAG AATATCAAGA | 200 |
| 201 | CTGTACCGAG TAGGGGGCCT TGGCAAGTGT GGAGAGCCCG GCAGCTGGGG | 250 |
| 251 | CAGAGGGCGG AGTACGGTGT GCGTTTACGG ACCTCTTCAA ACGAGGTAGG | 300 |
| 301 | AAGGTCAGAA GTCAAAAAGG GAACAAATGA TGTTTAACCA CACAAAAATG | 350 |
| 351 | AAAATCCAAT GGTTGGATAT CCATTCCAAA TACACAAAGG CAACGGATAA | 400 |
| 401 | GTGATCCGGG CCAGGCACAG AAGGCCATGC ACCCGTAGGA TTGCACTCAG | 450 |
| 451 | AGCTCCCAAA TGCATAGGAA TAGAAGGGTG GGTGCAGGAG GCTGAGGGGT | 500 |
| 501 | GGGGAAAGGG CATGGGTGTT TCATGAGGAC AGAGCTTCCG TTTCATGCAA | 550 |
| 551 | TGAAAAGAGT TTGGAGACGG ATGGTGGTGA CTGGACTATA CACTTACACA | 600 |
| 601 | CGGTAGCGAT GGTACACTTT GTATTATGTA TATTTTACCA CGATCTTTTT | 650 |
| 651 | AAAGTGTCAA AGGCAAATGG CCAAATGGTT CCTTGTCCTA TAGCTGTAGC | 700 |
| 701 | AGCCATCGGC TGTTAGTGAC AAAGCCCCTG AGTCAAGATG ACAGCAGCCC | 750 |
| 751 | CCATAACTCC TAATCGGCTC TCCCGCGTGG AGTCATTTAG GAGTAGTCGC | 800 |
| 801 | ATTAGAGACA AGTCCAACAT CTAATCTTCC ACCCTGGCCA GGGCCCCAGC | 850 |
| 851 | TGGCAGCGAG GGTGGGAGAC TCCGGGCAGA GCAGAGGGCG CTGACATTGG | 900 |
| 901 | GGCCCGGCCT GGCTTGGGTC CCTCTGGCCT TTCCCCAGGG GCCCTCTTTC | 950 |
| 951 | CTTGGGGCTT TCTTGGGCCG CCACTGCTCC CGCTCCTCTC CCCCCATCCC | 1000 |
| 1001 | ACCCCCTCAC CCCCTCGTTC TTCATATCCT TCTCTAGTGC TCCCTCCACT | 1050 |
| 1051 | TTCATCCACC CTTCTGCAAG AGTGTGGGAC CACAAATGAG TTTTCACCTG | 1100 |
| 1101 | GCCTGGGGAC ACACGTGCCC CCACAGGTGC TGAGTGACTT TCTAGGACAG | 1150 |
| 1151 | TAATCTGCTT TAGGCTAAAA TGGGACTTGA TCTTCTGTTA GCCCTAATCA | 1200 |
| 1201 | TCAATTAGCA GAGCCGGTGA AGGTGCAGAA CCTACCGCCT TTCCAGGCCT | 1250 |
| 1251 | CCTCCCACCT CTGCCACCTC CACTCTCCTT CCTGGGATGT GGGGGCTGGC | 1300 |
| 1301 | ACACGTGTGG CCCAGGGCAT TGGTGGGATT GCACTGAGCT GGGTCATTAG | 1350 |
| 1351 | CGTAATCCTG GACAAGGGCA GACAGGGCGA GCGGAGGGCC AGCTCCGGGG | 1400 |
| 1401 | CTCAGGCAAG GCTGGGGGCT TCCCCCAGAC ACCCCACTCC TCCTCTGCTG | 1450 |
| 1451 | GACCCCCACT TCATAGGGCA CTTCGTGTTC TCAAAGGGCT TCCAAATAGC | 1500 |
| 1501 | ATGGTGGCCT TGGATGCCCA GGGAAGCCTC AGAGTTGCTT ATCTCCCTCT | 1550 |
| 1551 | AGACAGAAGG GGAATCTCGG TCAAGAGGGA GAGGTCGCCC TGTTCAAGGC | 1600 |
| 1601 | CACCCAGCCA GCTCATGGCG GTAATGGGAC AAGGCTGGCC AGCCATCCCA | 1650 |
| 1651 | CCCTCAGAAG GGACCCGGTG GGGCAGGTGA TCTCAGAGGA GGCTCACTTC | 1700 |
| 1701 | TGGGTCTCAC ATTCTTCCAG CAAATCCCTC TGAGCCGCCC CCGGGGGCTC | 1750 |
| 1751 | GCCTCAGGAG CAAGGAAGCA AGGGGTGGGA GGAGGAGGTC TAAGTCCCAG | 1800 |
| 1801 | GCCCAATTAA GAGATCAGAT GGTGTAGGAT TTGGGAGCTT TTAAGGTGAA | 1850 |
| 1851 | GAGGCCCGGG CTGATCCCAC TGGCCGGTAT AAAGCACCGT GACCCTCAGG | 1900 |
| 1901 | TGACGCACCA GGGCCGGCTG CCGTCGGGGA CAGGGCTTTC CATAGCCCAG | 1950 |
| 1951 | GTAAGTATCA AGGTTACAAG ACAGGTTTAA GGAGACCAAT AGAAACTGGG | 2000 |
| 2001 | CTTGTCGAGA CAGAGAAGAC TCTTGCGTTT CTGATAGGCA CCTATTGGTC | 2050 |
| 2051 | TTACTGACAT CCACTTTGCC TTTCTCTCCA CAGGCCCAGA GAGGAGACAG | 2100 |
| 2101 | GCTAGCGCTA GCGCCGCCAC CATGTTCAAG AGCCTCACCA AAGTCAACAA | 2150 |
| 2151 | GGTCAAGCCA ATTGGAGAGA ACAATGAGAA CGAACAGTCC AGCAGACGGA | 2200 |
| 2201 | ATGAGGAGGG ATCCCACCCA TCCAACCAGA GCCAGCAGAC CACTGCCCAA | 2250 |
| 2251 | GAGGAAAACA AGGGAGAGGA AAAGTCCCTC AAGACCAAGT CCACCCCTGT | 2300 |
| 2301 | CACCTCTGAG GAACCCCACA CCAACATCCA GGACAAGCTG TCCAAGAAGA | 2350 |
| 2351 | ACTCCTCAGG AGACTTGACC ACCAACCCTG ACCCCCAAAA TGCAGCGGAG | 2400 |
| 2401 | CCCACAGGCA CTGTGCCGGA ACAGAAGGAA ATGGACCCGG GAAAGGAGGG | 2450 |
| 2451 | GCCTAACAGC CCTCAGAACA AGCCTCCAGC TGCCCCAGTG ATCAACGAAT | 2500 |
| 2501 | ATGCTGATGC CCAGCTTCAC AACCTGGTCA AGCGCATGAG ACAGAGGACT | 2550 |
| 2551 | GCCCTGTACA AGAAGAAGCT TGTGGAAGGG GACCTGTCCA GCCCTGAGGC | 2600 |
| 2601 | CTCCCCGCAA ACTGCCAAGC CCACGGCTGT GCCCCCTGTG AAAGAGTCGG | 2650 |
| 2651 | ATGACAAGCC CACTGAGCAT TACTACCGCC TGCTGTGGTT CAAAGTTAAG | 2700 |
| 2701 | AAGATGCCCC TCACTGAATA CCTGAAGCGC ATCAAGCTAC CTAACTCCAT | 2750 |
| 2751 | TGACTCATAC ACTGACCGGC TCTACTTGCT GTGGCTGCTG CTTGTGACCC | 2800 |
| 2801 | TTGCCTACAA CTGGAACTGC TGCTTCATCC CTCTGAGGCT GGTGTTCCCG | 2850 |
| 2851 | TACCAAACTG CAGACAACAT CCACTACTGG CTGATTGCTG ACATCATCTG | 2900 |
| 2901 | TGATATCATC TACCTCTATG ACATGCTGTT CATCCAACCA AGGCTGCAGT | 2950 |
| 2951 | TCGTGAGAGG GGGAGACATC ATTGTGGACT CCAATGAGCT CCGGAAGCAC | 3000 |
| 3001 | TACCGCACCT CCACCAAGTT CCAGCTGGAT GTGGCCTCCA TCATCCCCTT | 3050 |
| 3051 | TGACATCTGC TACCTGTTCT TTGGATTCAA CCCCATGTTC CGGGCCAACA | 3100 |
| 3101 | GAATGCTGAA GTACACCTCC TTCTTTGAAT TCAACCATCA CCTGGAATCC | 3150 |
| 3151 | ATCATGGACA AGGCCTACAT CTACCGGGTC ATCCGCACCA CTGGTTACCT | 3200 |
| 3201 | GTTGTTCATC CTGCACATAA ATGCCTGTGT CTACTATTGG GCCTCCAACT | 3250 |
| 3251 | ATGAAGGCAT TGGTACCACC AGATGGGTGT ATGATGGAGA GGGCAATGAG | 3300 |
| 3301 | TACCTCCGGT GCTACTACTG GGCAGTGCGC ACCCTGATCA CAATTGGGGG | 3350 |
| 3351 | CCTCCCTGAG CCCCAGACCC TGTTTGAAAT TGTGTTCCAA CTGCTGAACT | 3400 |
| 3401 | TCTTCTCGGG AGTGTTTGTG TTCAGCAGCC TCATTGGCCA GATGAGAGAT | 3450 |
| 3451 | GTCATTGGAG CAGCCACTGC CAACCAGAAC TACTTCAGGG CCTGCATGGA | 3500 |
| 3501 | TGACACCATT GCCTACATGA ACAACTACTC CATTCCCAAG CTTGTGCAGA | 3550 |
| 3551 | AGAGAGTGCG AACTTGGTAT GAGTACACCT GGGACTCCCA GAGGATGCTG | 3600 |
| 3601 | GATGAGTCAG ACTTACTCAA GACCCTGCCC ACCACTGTGC AGCTTGCCCT | 3650 |
| 3651 | GGCCATTGAT GTGAACTTCT CCATCATCTC CAAAGTGGAC CTGTTCAAGG | 3700 |
| 3701 | GCTGTGACAC CCAGATGATC TACGACATGT TGCTGCGGCT GAAGTCGGTG | 3750 |
| 3751 | CTCTACCTCC CTGGAGATTT TGTGTGCAAG AAGGGAGAAA TTGGGAAGGA | 3800 |
| 3801 | AATGTACATC ATCAAGCATG GAGAGGTCCA AGTGCTGGGT GGCCCGGATG | 3850 |
| 3851 | GCACCAAAGT GCTGGTCACC CTGAAGGCTG GCTCAGTGTT TGGAGAAATC | 3900 |
| 3901 | AGCCTCTTGG CGGCTGGGGG GGGCAACAGG AGAACTGCCA ATGTGGTAGC | 3950 |
| 3951 | CCATGGCTTT GCCAACCTCC TGACCCTTGA CAAGAAAACC CTCCAGGAAA | 4000 |
| 4001 | TCCTGGTGCA CTACCCGGAC TCAGAGAGAA TCCTGATGAA GAAGGCCCGG | 4050 |
| 4051 | GTGCTGCTGA AGCAGAAGGC CAAGACTGCA GAGGCCACCC CCCCACGCAA | 4100 |
| 4101 | AGACCTGGCC CTCCTGTTCC CGCCCAAGGA AGAAACCCCA AAGCTGTTCA | 4150 |
| 4151 | AGACCCTCCT GGGTGGCACT GGGAAGGCCT CCCTGGCCCG CCTGTTGAAG | 4200 |
| 4201 | CTCAAAAGGG AACAGGCAGC CCAGAAGAAG GAGAACTCAG AGGGGGGAGA | 4250 |
| 4251 | GGAAGAGGGC AAAGAGAACG AGGATAAGCA AAAGGAGAAC GAAGATAAGC | 4300 |
| 4301 | AGAAGGAAAA CGAGGACAAG GGAAAAGAAA ATGAGGACAA GGACAAGGGT | 4350 |
| 4351 | CGGGAGCCTG AAGAGAAGCC CCTGGACCGG CCTGAATGCA CTGCCAGCCC | 4400 |
| 4401 | CATTGCTGTG GAAGAAGAAC CCCACAGTGT CAGAAGGACT GTGCTGCCGA | 4450 |
| 4451 | GAGGCACCAG CCGGCAGTCC CTGATCATCA GCATGGCCCC TTCTGCGGAG | 4500 |
| 4501 | GGTGGAGAAG AAGTGCTGAC CATTGAAGTC AAGGAAAAGG CCAAGCAGTA | 4550 |
| 4551 | AGCGGCCGCA ATAAAAGATC TTTATTTTCA TTAGATCTGT GTGTTGGTTT | 4600 |
| 4601 | TTTGTGTGTA CGTAGTTAAT CATTAACTAC AAGGAACCCC TAGTGATGGA | 4650 |
| 4651 | GTTGGCCACT CCCTCTCTGC GCGCTCGCTC GCTCACTGAG GCCGGGCGAC | 4700 |
| 4701 | CAAAGGTCGC CCGACGCCCG GGCTTTGCCC GGGCGGCCTC AGTGAGCGAG | 4750 |
| 4751 | CGAGCGCGC | 4759 |

| Name | Start | End |
|---|---|---|
| ITR, inverse terminal repeat from AAV2 | 1 | 145 |
| LCR, human opsin locus control region | 152 | 1716 |
| OPN1LW promoter, human OPN1LW core promoter | 1717 | 1947 |
| Chimeric intron | 1948 | 2084 |
| 5′UTR | 2085 | 2112 |
| OK, optimized Kozak | 2113 | 2121 |
| hCNGB3 codon optimized V11(CNGB3coV11), | 2122 | 4551 |
| Codon optimized cDNA encoding human cyclic nucleotide | | |
| gated channel subunit beta 3 | | |
| SPA, short synthetic polyadenylation sequence | 4560 | 4608 |
| ITR, inverse terminal repeat from AAV2 | 4615 | 4759 |

| -pADV963-MNTC-5′UTR.Ctnnb1-CNGB3coV11-SPA | | |
|---|---|---|
| SEQ ID NO: 3 | | |
| 1 | GCGCGCTCGC TCGCTCACTG AGGCCGCCCG GGCAAAGCCC GGGCGTCGGG | 50 |
| 51 | CGACCTTTGG TCGCCCGGCC TCAGTGAGCG AGCGAGCGCG CAGAGAGGGA | 100 |
| 101 | GTGGCCAACT CCATCACTAG GGGTTCCTTG TAGTTAATGA TTAACACTAG | 150 |
| 151 | TCCTACAGCA GCCAGGGTGA GATTATGAGG CTGAGCTGAG AATATCAAGA | 200 |
| 201 | CTGTACCGAG TAGGGGGCCT TGGCAAGTGT GGAGAGCCCG GCAGCTGGGG | 250 |
| 251 | CAGAGGGCGG AGTACGGTGT GCGTTTACGG ACCTCTTCAA ACGAGGTAGG | 300 |
| 301 | AAGGTCAGAA GTCAAAAAGG GAACAAATGA TGTTTAACCA CACAAAAATG | 350 |
| 351 | AAAATCCAAT GGTTGGATAT CCATTCCAAA TACACAAAGG CAACGGATAA | 400 |
| 401 | GTGATCCGGG CCAGGCACAG AAGGCCATGC ACCCGTAGGA TTGCACTCAG | 450 |
| 451 | AGCTCCCAAA TGCATAGGAA TAGAAGGGTG GGTGCAGGAG GCTGAGGGGT | 500 |
| 501 | GGGGAAAGGG CATGGGTGTT TCATGAGGAC AGAGCTTCCG TTTCATGCAA | 550 |
| 551 | TGAAAAGAGT TTGGAGACGG ATGGTGGTGA CTGGACTATA CACTTACACA | 600 |
| 601 | CGGTAGCGAT GGTACACTTT GTATTATGTA TATTTTACCA CGATCTTTTT | 650 |
| 651 | AAAGTGTCAA AGGCAAATGG CCAAATGGTT CCTTGTCCTA TAGCTGTAGC | 700 |
| 701 | AGCCATCGGC TGTTAGTGAC AAAGCCCCTG AGTCAAGATG ACAGCAGCCC | 750 |
| 751 | CCATAACTCC TAATCGGCTC TCCCGCGTGG AGTCATTTAG GAGTAGTCGC | 800 |
| 801 | ATTAGAGACA AGTCCAACAT CTAATCTTCC ACCCTGGCCA GGGCCCCAGC | 850 |
| 851 | TGGCAGCGAG GGTGGGAGAC TCCGGGCAGA GCAGAGGGCG CTGACATTGG | 900 |
| 901 | GGCCCGGCCT GGCTTGGGTC CCTCTGGCCT TTCCCCAGGG GCCCTCTTTC | 950 |
| 951 | CTTGGGGCTT TCTTGGGCCG CCACTGCTCC CGCTCCTCTC CCCCCATCCC | 1000 |
| 1001 | ACCCCCTCAC CCCCTCGTTC TTCATATCCT TCTCTAGTGC TCCCTCCACT | 1050 |
| 1051 | TTCATCCACC CTTCTGCAAG AGTGTGGGAC CACAAATGAG TTTTCACCTG | 1100 |
| 1101 | GCCTGGGGAC ACACGTGCCC CCACAGGTGC TGAGTGACTT TCTAGGACAG | 1150 |
| 1151 | TAATCTGCTT TAGGCTAAAA TGGGACTTGA TCTTCTGTTA GCCCTAATCA | 1200 |
| 1201 | TCAATTAGCA GAGCCGGTGA AGGTGCAGAA CCTACCGCCT TTCCAGGCCT | 1250 |
| 1251 | CCTCCCACCT CTGCCACCTC CACTCTCCTT CCTGGGATGT GGGGGCTGGC | 1300 |
| 1301 | ACACGTGTGG CCCAGGGCAT TGGTGGGATT GCACTGAGCT GGGTCATTAG | 1350 |
| 1351 | CGTAATCCTG GACAAGGGCA GACAGGGCGA GCGGAGGGCC AGCTCCGGGG | 1400 |
| 1401 | CTCAGGCAAG GCTGGGGGCT TCCCCCAGAC ACCCCACTCC TCCTCTGCTG | 1450 |
| 1451 | GACCCCCACT TCATAGGGCA CTTCGTGTTC TCAAAGGGCT TCCAAATAGC | 1500 |
| 1501 | ATGGTGGCCT TGGATGCCCA GGGAAGCCTC AGAGTTGCTT ATCTCCCTCT | 1550 |
| 1551 | AGACAGAAGG GGAATCTCGG TCAAGAGGGA GAGGTCGCCC TGTTCAAGGC | 1600 |
| 1601 | CACCCAGCCA GCTCATGGCG GTAATGGGAC AAGGCTGGCC AGCCATCCCA | 1650 |
| 1651 | CCCTCAGAAG GGACCCGGTG GGGCAGGTGA TCTCAGAGGA GGCTCACTTC | 1700 |
| 1701 | TGGGTCTCAC ATTCTTCCAG CAAATCCCTC TGAGCCGCCC CCGGGGGCTC | 1750 |
| 1751 | GCCTCAGGAG CAAGGAAGCA AGGGGTGGGA GGAGGAGGTC TAAGTCCCAG | 1800 |
| 1801 | GCCCAATTAA GAGATCAGAT GGTGTAGGAT TTGGGAGCTT TTAAGGTGAA | 1850 |
| 1851 | GAGGCCCGGG CTGATCCCAC TGGCCGGTAT AAAGCACCGT GACCCTCAGG | 1900 |
| 1901 | TGACGCACCA GGGCCGGCTG CCGTCGGGGA CAGGGCTTTC CATAGCCCAG | 1950 |
| 1951 | GTAAGTATCA AGGTTACAAG ACAGGTTTAA GGAGACCAAT AGAAACTGGG | 2000 |
| 2001 | CTTGTCGAGA CAGAGAAGAC TCTTGCGTTT CTGATAGGCA CCTATTGGTC | 2050 |
| 2051 | TTACTGACAT CCACTTTGCC TTTCTCTCCA CAGGCCGGTG GCGGCAGGAT | 2100 |
| 2101 | ACAGCGGCTT CTGCGCGACT TATAAGAGCT CCTTGTGCGG CGCCATTTTA | 2150 |
| 2151 | AGCCTCTCGG TCTGTGGCAG CAGCGTTGGC CCGGCCGCCA CCATGTTCAA | 2200 |
| 2201 | GAGCCTCACC AAAGTCAACA AGGTCAAGCC AATTGGAGAG AACAATGAGA | 2250 |
| 2251 | ACGAACAGTC CAGCAGACGG AATGAGGAGG GATCCCACCC ATCCAACCAG | 2300 |
| 2301 | AGCCAGCAGA CCACTGCCCA AGAGGAAAAC AAGGGAGAGG AAAAGTCCCT | 2350 |
| 2351 | CAAGACCAAG TCCACCCCTG TCACCTCTGA GGAACCCCAC ACCAACATCC | 2400 |
| 2401 | AGGACAAGCT GTCCAAGAAG AACTCCTCAG GAGACTTGAC CACCAACCCT | 2450 |
| 2451 | GACCCCCAAA ATGCAGCGGA GCCCACAGGC ACTGTGCCGG AACAGAAGGA | 2500 |
| 2501 | AATGGACCCG GGAAAGGAGG GGCCTAACAG CCCTCAGAAC AAGCCTCCAG | 2550 |
| 2551 | CTGCCCCAGT GATCAACGAA TATGCTGATG CCCAGCTTCA CAACCTGGTC | 2600 |
| 2601 | AAGCGCATGA GACAGAGGAC TGCCCTGTAC AAGAAGAAGC TTGTGGAAGG | 2650 |
| 2651 | GGACCTGTCC AGCCCTGAGG CCTCCCCGCA AACTGCCAAG CCCACGGCTG | 2700 |
| 2701 | TGCCCCCTGT GAAAGAGTCG GATGACAAGC CCACTGAGCA TTACTACCGC | 2750 |
| 2751 | CTGCTGTGGT TCAAAGTTAA GAAGATGCCC CTCACTGAAT ACCTGAAGCG | 2800 |
| 2801 | CATCAAGCTA CCTAACTCCA TTGACTCATA CACTGACCGG CTCTACTTGC | 2850 |
| 2851 | TGTGGCTGCT GCTTGTGACC CTTGCCTACA ACTGGAACTG CTGCTTCATC | 2900 |
| 2901 | CCTCTGAGGC TGGTGTTCCC GTACCAAACT GCAGACAACA TCCACTACTG | 2950 |
| 2951 | GCTGATTGCT GACATCATCT GTGATATCAT CTACCTCTAT GACATGCTGT | 3000 |
| 3001 | TCATCCAACC AAGGCTGCAG TTCGTGAGAG GGGGAGACAT CATTGTGGAC | 3050 |
| 3051 | TCCAATGAGC TCCGGAAGCA CTACCGCACC TCCACCAAGT TCCAGCTGGA | 3100 |
| 3101 | TGTGGCCTCC ATCATCCCCT TTGACATCTG CTACCTGTTC TTTGGATTCA | 3150 |
| 3151 | ACCCCATGTT CCGGGCCAAC AGAATGCTGA AGTACACCTC CTTCTTTGAA | 3200 |
| 3201 | TTCAACCATC ACCTGGAATC CATCATGGAC AAGGCCTACA TCTACCGGGT | 3250 |
| 3251 | CATCCGCACC ACTGGTTACC TGTTGTTCAT CCTGCACATA AATGCCTGTG | 3300 |
| 3301 | TCTACTATTG GGCCTCCAAC TATGAAGGCA TTGGTACCAC CAGATGGGTG | 3350 |
| 3351 | TATGATGGAG AGGGCAATGA GTACCTCCGG TGCTACTACT GGGCAGTGCG | 3400 |
| 3401 | CACCCTGATC ACAATTGGGG GCCTCCCTGA GCCCCAGACC CTGTTTGAAA | 3450 |
| 3451 | TTGTGTTCCA ACTGCTGAAC TTCTTCTCGG GAGTGTTTGT GTTCAGCAGC | 3500 |
| 3501 | CTCATTGGCC AGATGAGAGA TGTCATTGGA GCAGCCACTG CCAACCAGAA | 3550 |
| 3551 | CTACTTCAGG GCCTGCATGG ATGACACCAT TGCCTACATG AACAACTACT | 3600 |
| 3601 | CCATTCCCAA GCTTGTGCAG AAGAGAGTGC GAACTTGGTA TGAGTACACC | 3650 |
| 3651 | TGGGACTCCC AGAGGATGCT GGATGAGTCA GACTTACTCA AGACCCTGCC | 3700 |
| 3701 | CACCACTGTG CAGCTTGCCC TGGCCATTGA TGTGAACTTC TCCATCATCT | 3750 |
| 3751 | CCAAAGTGGA CCTGTTCAAG GGCTGTGACA CCCAGATGAT CTACGACATG | 3800 |
| 3801 | TTGCTGCGGC TGAAGTCGGT GCTCTACCTC CCTGGAGATT TTGTGTGCAA | 3850 |
| 3851 | GAAGGGAGAA ATTGGGAAGG AAATGTACAT CATCAAGCAT GGAGAGGTCC | 3900 |
| 3901 | AAGTGCTGGG TGGCCCGGAT GGCACCAAAG TGCTGGTCAC CCTGAAGGCT | 3950 |
| 3951 | GGCTCAGTGT TTGGAGAAAT CAGCCTCTTG GCGGCTGGGG GGGGCAACAG | 4000 |
| 4001 | GAGAACTGCC AATGTGGTAG CCCATGGCTT TGCCAACCTC CTGACCCTTG | 4050 |
| 4051 | ACAAGAAAAC CCTCCAGGAA ATCCTGGTGC ACTACCCGGA CTCAGAGAGA | 4100 |
| 4101 | ATCCTGATGA AGAAGGCCCG GGTGCTGCTG AAGCAGAAGG CCAAGACTGC | 4150 |
| 4151 | AGAGGCCACC CCCCCACGCA AAGACCTGGC CCTCCTGTTC CCGCCCAAGG | 4200 |
| 4201 | AAGAAACCCC AAAGCTGTTC AAGACCCTCC TGGGTGGCAC TGGGAAGGCC | 4250 |
| 4251 | TCCCTGGCCC GCCTGTTGAA GCTCAAAAGG GAACAGGCAG CCCAGAAGAA | 4300 |
| 4301 | GGAGAACTCA GAGGGGGGAG AGGAAGAGGG CAAAGAGAAC GAGGATAAGC | 4350 |
| 4351 | AAAAGGAGAA CGAAGATAAG CAGAAGGAAA ACGAGGACAA GGGAAAAGAA | 4400 |
| 4401 | AATGAGGACA AGGACAAGGG TCGGGAGCCT GAAGAGAAGC CCCTGGACCG | 4450 |
| 4451 | GCCTGAATGC ACTGCCAGCC CCATTGCTGT GGAAGAAGAA CCCCACAGTG | 4500 |
| 4501 | TCAGAAGGAC TGTGCTGCCG AGAGGCACCA GCCGGCAGTC CCTGATCATC | 4550 |
| 4551 | AGCATGGCCC CTTCTGCGGA GGGTGGAGAA GAAGTGCTGA CCATTGAAGT | 4600 |
| 4601 | CAAGGAAAAG GCCAAGCAGT AAGCGGCCGC AATAAAAGAT CTTTATTTTC | 4650 |
| 4651 | ATTAGATCTG TGTGTTGGTT TTTTGTGTGT ACGTAGTTAA TCATTAACTA | 4700 |
| 4701 | CAAGGAACCC CTAGTGATGG AGTTGGCCAC TCCCTCTCTG CGCGCTCGCT | 4750 |
| 4751 | CGCTCACTGA GGCCGGGCGA CCAAAGGTCG CCCGACGCCC GGGCTTTGCC | 4800 |
| 4801 | CGGGCGGCCT CAGTGAGCGA GCGAGCGCGC | 4830 |

| Name | Start | End |
|---|---|---|
| ITR, inverse terminal repeat from AAV2 | 1 | 145 |
| LCR, human opsin locus control region | 152 | 1716 |
| OPN1LW promoter, human OPN1LW core promoter | 1717 | 1947 |
| Chimeric intron | 1948 | 2084 |
| CTNNB1 5′UTR, human beta catenin 1 5′UTR | 2085 | 2183 |
| OK, optimized Kozak | 2184 | 2192 |
| hCNGB3 codon optimized V11(CNGB3coV11), | 2193 | 4622 |
| codon optimized cDNA encoding human cyclic nucleotide | | |
| gated channel subunit beta 3 | | |
| SPA, short synthetic polyadenylation sequence | 4631 | 4679 |
| ITR, inverse terminal repeat from AAV2 | 4686 | 4830 |

| -pADV854-PR1.7-CNGB3coV11-SV40pA | | |
|---|---|---|
| SEQ ID NO: 4 | | |
| 1 | GCGCGCTCGC TCGCTCACTG AGGCCGCCCG GGCAAAGCCC GGGCGTCGGG | 50 |
| 51 | CGACCTTTGG TCGCCCGGCC TCAGTGAGCG AGCGAGCGCG CAGAGAGGGA | 100 |
| 101 | GTGGCCAACT CCATCACTAG GGGTTCCTTG TAGTTAATGA TTAACACTAG | 150 |
| 151 | TGGAGGCTGA GGGGTGGGGA AAGGGCATGG GTGTTTCATG AGGACAGAGC | 200 |
| 201 | TTCCGTTTCA TGCAATGAAA AGAGTTTGGA GACGGATGGT GGTGACTGGA | 250 |
| 251 | CTATACACTT ACACACGGTA GCGATGGTAC ACTTTGTATT ATGTATATTT | 300 |
| 301 | TACCACGATC TTTTTAAAGT GTCAAAGGCA AATGGCCAAA TGGTTCCTTG | 350 |
| 351 | TCCTATAGCT GTAGCAGCCA TCGGCTGTTA GTGACAAAGC CCCTGAGTCA | 400 |
| 401 | AGATGACAGC AGCCCCCATA ACTCCTAATC GGCTCTCCCG CGTGGAGTCA | 450 |
| 451 | TTTAGGAGTA GTCGCATTAG AGACAAGTCC AACATCTAAT CTTCCACCCT | 500 |
| 501 | GGCCAGGGCC CCAGCTGGCA GCGAGGGTGG GAGACTCCGG GCAGAGCAGA | 550 |
| 551 | GGGCGCTGAC ATTGGGGCCC GGCCTGGCTT GGGTCCCTCT GGCCTTTCCC | 600 |
| 601 | CAGGGGCCCT CTTTCCTTGG GGCTTTCTTG GGCCGCCACT GCTCCCGCTC | 650 |
| 651 | CTCTCCCCCC ATCCCACCCC CTCACCCCCT CGTTCTTCAT ATCCTTCTCT | 700 |
| 701 | AGTGCTCCCT CCACTTTCAT CCACCCTTCT GCAAGAGTGT GGGACCACAA | 750 |
| 751 | ATGAGTTTTC ACCTGGCCTG GGGACACACG TGCCCCCACA GGTGCTGAGT | 800 |
| 801 | GACTTTCTAG GACAGTAATC TGCTTTAGGC TAAAATGGGA CTTGATCTTC | 850 |
| 851 | TGTTAGCCCT AATCATCAAT TAGCAGAGCC GGTGAAGGTG CAGAACCTAC | 900 |
| 901 | CGCCTTTCCA GGCCTCCTCC CACCTCTGCC ACCTCCACTC TCCTTCCTGG | 950 |
| 951 | GATGTGGGGG CTGGCACACG TGTGGCCCAG GGCATTGGTG GGATTGCACT | 1000 |
| 1001 | GAGCTGGGTC ATTAGCGTAA TCCTGGACAA GGGCAGACAG GGCGAGCGGA | 1050 |
| 1051 | GGGCCAGCTC CGGGGCTCAG GCAAGGCTGG GGGCTTCCCC CAGACACCCC | 1100 |
| 1101 | ACTCCTCCTC TGCTGGACCC CCACTTCATA GGGCACTTCG TGTTCTCAAA | 1150 |
| 1151 | GGGCTTCCAA ATAGCATGGT GGCCTTGGAT GCCCAGGGAA GCCTCAGAGT | 1200 |
| 1201 | TGCTTATCTC CCTCTAGACA GAAGGGGAAT CTCGGTCAAG AGGGAGAGGT | 1250 |
| 1251 | CGCCCTGTTC AAGGCCACCC AGCCAGCTCA TGGCGGTAAT GGGACAAGGC | 1300 |
| 1301 | TGGCCAGCCA TCCCACCCTC AGAAGGGACC CGGTGGGGCA GGTGATCTCA | 1350 |
| 1351 | GAGGAGGCTC ACTTCTGGGT CTCACATTCT TGGATCCGGT TCCAGGCCTC | 1400 |
| 1401 | GGCCCTAAAT AGTCTCCCTG GGCTTTCAAG AGAACCACAT GAGAAAGGAG | 1450 |
| 1451 | GATTCGGGCT CTGAGCAGTT TCACCACCCA CCCCCCAGTC TGCAAATCCT | 1500 |
| 1501 | GACCCGTGGG TCCACCTGCC CCAAAGGCGG ACGCAGGACA GTAGAAGGGA | 1550 |
| 1551 | ACAGAGAACA CATAAACACA GAGAGGGCCA CAGCGGCTCC CACAGTCACC | 1600 |
| 1601 | GCCACCTTCC TGGCGGGGAT GGGTGGGGCG TCTGAGTTTG GTTCCCAGCA | 1650 |
| 1651 | AATCCCTCTG AGCCGCCCTT GCGGGCTCGC CTCAGGAGCA GGGGAGCAAG | 1700 |
| 1701 | AGGTGGGAGG AGGAGGTCTA AGTCCCAGGC CCAATTAAGA GATCAGGTAG | 1750 |
| 1751 | TGTAGGGTTT GGGAGCTTTT AAGGTGAAGA GGCCCGGGCT GATCCCACAG | 1800 |
| 1801 | GCCAGTATAA AGCGCCGTGA CCCTCAGGTG ATGCGCCAGG GCCGGCTGCC | 1850 |
| 1851 | GTCGGGGACA GGGCTTTCCA TAGCCATGGC CACCATGTTC AAGAGCCTCA | 1900 |
| 1901 | CCAAAGTCAA CAAGGTCAAG CCAATTGGAG AGAACAATGA GAACGAACAG | 1950 |
| 1951 | TCCAGCAGAC GGAATGAGGA GGGATCCCAC CCATCCAACC AGAGCCAGCA | 2000 |
| 2001 | GACCACTGCC CAAGAGGAAA ACAAGGGAGA GGAAAAGTCC CTCAAGACCA | 2050 |
| 2051 | AGTCCACCCC TGTCACCTCT GAGGAACCCC ACACCAACAT CCAGGACAAG | 2100 |
| 2101 | CTGTCCAAGA AGAACTCCTC AGGAGACTTG ACCACCAACC CTGACCCCCA | 2150 |
| 2151 | AAATGCAGCG GAGCCCACAG GCACTGTGCC GGAACAGAAG GAAATGGACC | 2200 |
| 2201 | CGGGAAAGGA GGGGCCTAAC AGCCCTCAGA ACAAGCCTCC AGCTGCCCCA | 2250 |
| 2251 | GTGATCAACG AATATGCTGA TGCCCAGCTT CACAACCTGG TCAAGCGCAT | 2300 |
| 2301 | GAGACAGAGG ACTGCCCTGT ACAAGAAGAA GCTTGTGGAA GGGGACCTGT | 2350 |
| 2351 | CCAGCCCTGA GGCCTCCCCG CAAACTGCCA AGCCCACGGC TGTGCCCCCT | 2400 |
| 2401 | GTGAAAGAGT CGGATGACAA GCCCACTGAG CATTACTACC GCCTGCTGTG | 2450 |
| 2451 | GTTCAAAGTT AAGAAGATGC CCCTCACTGA ATACCTGAAG CGCATCAAGC | 2500 |
| 2501 | TACCTAACTC CATTGACTCA TACACTGACC GGCTCTACTT GCTGTGGCTG | 2550 |
| 2551 | CTGCTTGTGA CCCTTGCCTA CAACTGGAAC TGCTGCTTCA TCCCTCTGAG | 2600 |
| 2601 | GCTGGTGTTC CCGTACCAAA CTGCAGACAA CATCCACTAC TGGCTGATTG | 2650 |
| 2651 | CTGACATCAT CTGTGATATC ATCTACCTCT ATGACATGCT GTTCATCCAA | 2700 |
| 2701 | CCAAGGCTGC AGTTCGTGAG AGGGGGAGAC ATCATTGTGG ACTCCAATGA | 2750 |
| 2751 | GCTCCGGAAG CACTACCGCA CCTCCACCAA GTTCCAGCTG GATGTGGCCT | 2800 |
| 2801 | CCATCATCCC CTTTGACATC TGCTACCTGT TCTTTGGATT CAACCCCATG | 2850 |
| 2851 | TTCCGGGCCA ACAGAATGCT GAAGTACACC TCCTTCTTTG AATTCAACCA | 2900 |
| 2901 | TCACCTGGAA TCCATCATGG ACAAGGCCTA CATCTACCGG GTCATCCGCA | 2950 |
| 2951 | CCACTGGTTA CCTGTTGTTC ATCCTGCACA TAAATGCCTG TGTCTACTAT | 3000 |
| 3001 | TGGGCCTCCA ACTATGAAGG CATTGGTACC ACCAGATGGG TGTATGATGG | 3050 |
| 3051 | AGAGGGCAAT GAGTACCTCC GGTGCTACTA CTGGGCAGTG CGCACCCTGA | 3100 |
| 3101 | TCACAATTGG GGGCCTCCCT GAGCCCCAGA CCCTGTTTGA AATTGTGTTC | 3150 |
| 3151 | CAACTGCTGA ACTTCTTCTC GGGAGTGTTT GTGTTCAGCA GCCTCATTGG | 3200 |
| 3201 | CCAGATGAGA GATGTCATTG GAGCAGCCAC TGCCAACCAG AACTACTTCA | 3250 |
| 3251 | GGGCCTGCAT GGATGACACC ATTGCCTACA TGAACAACTA CTCCATTCCC | 3300 |
| 3301 | AAGCTTGTGC AGAAGAGAGT GCGAACTTGG TATGAGTACA CCTGGGACTC | 3350 |
| 3551 | CCAGAGGATG CTGGATGAGT CAGACTTACT CAAGACCCTG CCCACCACTG | 3400 |
| 3401 | TGCAGCTTGC CCTGGCCATT GATGTGAACT TCTCCATCAT CTCCAAAGTG | 4350 |
| 3451 | GACCTGTTCA AGGGCTGTGA CACCCAGATG ATCTACGACA TGTTGCTGCG | 3500 |
| 3501 | GCTGAAGTCG GTGCTCTACC TCCCTGGAGA TTTTGTGTGC AAGAAGGGAG | 3550 |
| 3551 | AAATTGGGAA GGAAATGTAC ATCATCAAGC ATGGAGAGGT CCAAGTGCTG | 3600 |
| 3601 | GGTGGCCCGG ATGGCACCAA AGTGCTGGTC ACCCTGAAGG CTGGCTCAGT | 3650 |
| 3651 | GTTTGGAGAA ATCAGCCTCT TGGCGGCTGG GGGGGGCAAC AGGAGAACTG | 3700 |
| 3701 | CCAATGTGGT AGCCCATGGC TTTGCCAACC TCCTGACCCT TGACAAGAAA | 3750 |
| 3751 | ACCCTCCAGG AAATCCTGGT GCACTACCCG GACTCAGAGA GAATCCTGAT | 3800 |
| 3801 | GAAGAAGGCC CGGGTGCTGC TGAAGCAGAA GGCCAAGACT GCAGAGGCCA | 3850 |
| 3851 | CCCCCCCACG CAAAGACCTG GCCCTCCTGT TCCCGCCCAA GGAAGAAACC | 3900 |
| 3901 | CCAAAGCTGT TCAAGACCCT CCTGGGTGGC ACTGGGAAGG CCTCCCTGGC | 3950 |
| 3951 | CCGCCTGTTG AAGCTCAAAA GGGAACAGGC AGCCCAGAAG AAGGAGAACT | 4000 |
| 4001 | CAGAGGGGGG AGAGGAAGAG GGCAAAGAGA ACGAGGATAA GCAAAAGGAG | 4050 |
| 4051 | AACGAAGATA AGCAGAAGGA AAACGAGGAC AAGGGAAAAG AAAATGAGGA | 4100 |
| 4101 | CAAGGACAAG GGTCGGGAGC CTGAAGAGAA GCCCCTGGAC CGGCCTGAAT | 4150 |
| 4151 | GCACTGCCAG CCCCATTGCT GTGGAAGAAG AACCCCACAG TGTCAGAAGG | 4200 |
| 4201 | ACTGTGCTGC CGAGAGGCAC CAGCCGGCAG TCCCTGATCA TCAGCATGGC | 4250 |
| 4251 | CCCTTCTGCG GAGGGTGGAG AAGAAGTGCT GACCATTGAA GTCAAGGAAA | 4300 |
| 4301 | AGGCCAAGCA GTAAGCGGCC GCGCGGCCGC TTCGAGCAGA CATGATAAGA | 4350 |
| 4351 | TACATTGATG AGTTTGGACA AACCACAACT AGAATGCAGT GAAAAAAATG | 4400 |
| 4401 | CTTTATTTGT GAAATTTGTG ATGCTATTGC TTTATTTGTA ACCATTATAA | 4450 |
| 4451 | GCTGCAATAA ACAAGTTAAC AACAACAATT GCATTCATTT TATGTTTCAG | 4500 |
| 4501 | GTTCAGGGGG AGATGTGGGA GGTTTTTTAA AGCAAGTAAA ACCTCTACAA | 4550 |
| 4551 | ATGTGGTAAA ATCGATAAGG ATCCTACGTA GTTAATCATT AACTACAAGG | 4600 |
| 4601 | AACCCCTAGT GATGGAGTTG GCCACTCCCT CTCTGCGCGC TCGCTCGCTC | 4650 |
| 4651 | ACTGAGGCCG GGCGACCAAA GGTCGCCCGA CGCCCGGGCT TTGCCCGGGC | 4700 |
| 4701 | GGCCTCAGTG AGCGAGCGAG CGCGC | 4725 |

| Name | Start | End |
|---|---|---|
| ITR, inverse terminal repeat from AAV2 | 1 | 145 |
| PR1.7 promoter | 152 | 1877 |
| OK, optimized Kozak | 1878 | 1884 |
| hCNGB3 codon optimized V11(CNGB3coV11), | 1885 | 4314 |
| codon optimized cDNA encoding human cyclic nucleotide | | |
| gated channel subunit beta 3 | | |
| SV40pA, simian virus 40 polyadenylation sequence | 4337 | 4573 |
| ITR, inverse terminal repeat from AAV2 | 4581 | 4725 |

| -pADV843-MNTC-eGFP-SV40pA | | |
|---|---|---|
| SEQ ID NO: 5 | | |
| 1 | GCGCGCTCGC TCGCTCACTG AGGCCGCCCG GGCAAAGCCC GGGCGTCGGG | 50 |
| 51 | CGACCTTTGG TCGCCCGGCC TCAGTGAGCG AGCGAGCGCG CAGAGAGGGA | 100 |
| 101 | GTGGCCAACT CCATCACTAG GGGTTCCTTG TAGTTAATGA TTAACACTAG | 150 |
| 151 | TCCTACAGCA GCCAGGGTGA GATTATGAGG CTGAGCTGAG AATATCAAGA | 200 |
| 201 | CTGTACCGAG TAGGGGGCCT TGGCAAGTGT GGAGAGCCCG GCAGCTGGGG | 250 |
| 251 | CAGAGGGCGG AGTACGGTGT GCGTTTACGG ACCTCTTCAA ACGAGGTAGG | 300 |
| 301 | AAGGTCAGAA GTCAAAAAGG GAACAAATGA TGTTTAACCA CACAAAAATG | 350 |
| 351 | AAAATCCAAT GGTTGGATAT CCATTCCAAA TACACAAAGG CAACGGATAA | 400 |
| 401 | GTGATCCGGG CCAGGCACAG AAGGCCATGC ACCCGTAGGA TTGCACTCAG | 450 |
| 451 | AGCTCCCAAA TGCATAGGAA TAGAAGGGTG GGTGCAGGAG GCTGAGGGGT | 500 |
| 501 | GGGGAAAGGG CATGGGTGTT TCATGAGGAC AGAGCTTCCG TTTCATGCAA | 550 |
| 551 | TGAAAAGAGT TTGGAGACGG ATGGTGGTGA CTGGACTATA CACTTACACA | 600 |
| 601 | CGGTAGCGAT GGTACACTTT GTATTATGTA TATTTTACCA CGATCTTTTT | 650 |
| 651 | AAAGTGTCAA AGGCAAATGG CCAAATGGTT CCTTGTCCTA TAGCTGTAGC | 700 |
| 701 | AGCCATCGGC TGTTAGTGAC AAAGCCCCTG AGTCAAGATG ACAGCAGCCC | 750 |
| 751 | CCATAACTCC TAATCGGCTC TCCCGCGTGG AGTCATTTAG GAGTAGTCGC | 800 |
| 801 | ATTAGAGACA AGTCCAACAT CTAATCTTCC ACCCTGGCCA GGGCCCCAGC | 850 |
| 851 | TGGCAGCGAG GGTGGGAGAC TCCGGGCAGA GCAGAGGGCG CTGACATTGG | 900 |
| 901 | GGCCCGGCCT GGCTTGGGTC CCTCTGGCCT TTCCCCAGGG GCCCTCTTTC | 950 |
| 951 | CTTGGGGCTT TCTTGGGCCG CCACTGCTCC CGCTCCTCTC CCCCCATCCC | 1000 |
| 1001 | ACCCCCTCAC CCCCTCGTTC TTCATATCCT TCTCTAGTGC TCCCTCCACT | 1050 |
| 1051 | TTCATCCACC CTTCTGCAAG AGTGTGGGAC CACAAATGAG TTTTCACCTG | 1100 |
| 1101 | GCCTGGGGAC ACACGTGCCC CCACAGGTGC TGAGTGACTT TCTAGGACAG | 1150 |
| 1151 | TAATCTGCTT TAGGCTAAAA TGGGACTTGA TCTTCTGTTA GCCCTAATCA | 1200 |
| 1201 | TCAATTAGCA GAGCCGGTGA AGGTGCAGAA CCTACCGCCT TTCCAGGCCT | 1250 |
| 1251 | CCTCCCACCT CTGCCACCTC CACTCTCCTT CCTGGGATGT GGGGGCTGGC | 1300 |
| 1301 | ACACGTGTGG CCCAGGGCAT TGGTGGGATT GCACTGAGCT GGGTCATTAG | 1350 |
| 1351 | CGTAATCCTG GACAAGGGCA GACAGGGCGA GCGGAGGGCC AGCTCCGGGG | 1400 |
| 1401 | CTCAGGCAAG GCTGGGGGCT TCCCCCAGAC ACCCCACTCC TCCTCTGCTG | 1450 |
| 1451 | GACCCCCACT TCATAGGGCA CTTCGTGTTC TCAAAGGGCT TCCAAATAGC | 1500 |
| 1501 | ATGGTGGCCT TGGATGCCCA GGGAAGCCTC AGAGTTGCTT ATCTCCCTCT | 1550 |
| 1551 | AGACAGAAGG GGAATCTCGG TCAAGAGGGA GAGGTCGCCC TGTTCAAGGC | 1600 |
| 1601 | CACCCAGCCA GCTCATGGCG GTAATGGGAC AAGGCTGGCC AGCCATCCCA | 1650 |
| 1651 | CCCTCAGAAG GGACCCGGTG GGGCAGGTGA TCTCAGAGGA GGCTCACTTC | 1700 |
| 1701 | TGGGTCTCAC ATTCTTCCAG CAAATCCCTC TGAGCCGCCC CCGGGGGCTC | 1750 |
| 1751 | GCCTCAGGAG CAAGGAAGCA AGGGGTGGGA GGAGGAGGTC TAAGTCCCAG | 1800 |
| 1801 | GCCCAATTAA GAGATCAGAT GGTGTAGGAT TTGGGAGCTT TTAAGGTGAA | 1850 |
| 1851 | GAGGCCCGGG CTGATCCCAC TGGCCGGTAT AAAGCACCGT GACCCTCAGG | 1900 |
| 1901 | TGACGCACCA GGGCCGGCTG CCGTCGGGGA CAGGGCTTTC CATAGCCCAG | 1950 |
| 1951 | GTAAGTATCA AGGTTACAAG ACAGGTTTAA GGAGACCAAT AGAAACTGGG | 2000 |
| 2001 | CTTGTCGAGA CAGAGAAGAC TCTTGCGTTT CTGATAGGCA CCTATTGGTC | 2050 |
| 2051 | TTACTGACAT CCACTTTGCC TTTCTCTCCA CAGGCCCAGA GAGGAGACAG | 2100 |
| 2101 | GCTAGCGCCG CCACCATGGT GAGCAAGGGC GAGGAGCTGT TCACCGGGGT | 2150 |
| 2151 | GGTGCCCATC CTGGTCGAGC TGGACGGCGA CGTAAACGGC CACAAGTTCA | 2200 |
| 2201 | GCGTGTCCGG CGAGGGCGAG GGCGATGCCA CCTACGGCAA GCTGACCCTG | 2250 |
| 2251 | AAGTTCATCT GCACCACCGG CAAGCTGCCC GTGCCCTGGC CCACCCTCGT | 2300 |
| 2301 | GACCACCCTG ACCTACGGCG TGCAGTGCTT CAGCCGCTAC CCCGACCACA | 2350 |
| 2351 | TGAAGCAGCA CGACTTCTTC AAGTCCGCCA TGCCCGAAGG CTACGTCCAG | 2400 |
| 2401 | GAGCGCACCA TCTTCTTCAA GGACGACGGC AACTACAAGA CCCGCGCCGA | 2450 |
| 2451 | GGTGAAGTTC GAGGGCGACA CCCTGGTGAA CCGCATCGAG CTGAAGGGCA | 2500 |
| 2501 | TCGACTTCAA GGAGGACGGC AACATCCTGG GGCACAAGCT GGAGTACAAC | 2550 |
| 2551 | TACAACAGCC ACAACGTCTA TATCATGGCC GACAAGCAGA AGAACGGCAT | 2600 |
| 2601 | CAAGGTGAAC TTCAAGATCC GCCACAACAT CGAGGACGGC AGCGTGCAGC | 2650 |
| 2651 | TCGCCGACCA CTACCAGCAG AACACCCCCA TCGGCGACGG CCCCGTGCTG | 2700 |
| 2701 | CTGCCCGACA ACCACTACCT GAGCACCCAG TCCGCCCTGA GCAAAGACCC | 2750 |
| 2751 | CAACGAGAAG CGCGATCACA TGGTCCTGCT GGAGTTCGTG ACCGCCGCCG | 2800 |
| 2801 | GGATCACTCT CGGCATGGAC GAGCTGTACA AGTAAGCGGC CGCTTCGAGC | 2850 |
| 2851 | AGACATGATA AGATACATTG ATGAGTTTGG ACAAACCACA ACTAGAATGC | 2900 |
| 2901 | AGTGAAAAAA ATGCTTTATT TGTGAAATTT GTGATGCTAT TGCTTTATTT | 2950 |
| 2951 | GTAACCATTA TAAGCTGCAA TAAACAAGTT AACAACAACA ATTGCATTCA | 3000 |
| 3001 | TTTTATGTTT CAGGTTCAGG GGGAGATGTG GGAGGTTTTT TAAAGCAAGT | 3050 |
| 3051 | AAAACCTCTA CAAATGTGGT AAAATCGATA AGGATCTACG TAGTTAATCA | 3100 |
| 3101 | TTAACTACAA GGAACCCCTA GTGATGGAGT TGGCCACTCC CTCTCTGCGC | 3150 |
| 3151 | GCTCGCTCGC TCACTGAGGC CGGGCGACCA AAGGTCGCCC GACGCCCGGG | 3200 |
| 3201 | CTTTGCCCGG GCGGCCTCAG TGAGCGAGCG AGCGCGC | 3237 |

| Name | Start | End |
|---|---|---|
| ITR, inverse terminal repeat from AAV2 | 1 | 145 |
| LCR, human opsin locus control region | 152 | 1716 |
| OPN1LW promoter, human OPN1LW core promoter | 1717 | 1947 |
| Chimeric intron | 1948 | 2084 |
| 5′UTR | 2085 | 2106 |
| OK, optimized Kozak | 2107 | 2115 |
| eGFP, cDNA enhanced Green Fluorescent Protein | 2116 | 2835 |
| SV40pA, simian virus 40 polyadenylation sequence | 2850 | 3086 |
| ITR, inverse terminal repeat from AAV2 | 3093 | 3237 |

| SEQ ID NO: 6 | | |
|---|---|---|
| 1 | LALGETTRPA | 10 |
| SEQ ID NO: 7 | | |
| MAADGYLPDWLEDTLSEGIRQWWKLKPGPPPPKPAERHKDDSRGLVLPGYKYLGPFNGLD | | |
| KGEPVNEADAAALEHDKAYDRQLDSGDNPYLKYNHADAEFQERLKEDTSFGGNLGRAVFQ | | |
| AKKRVLEPLGLVEEPVKTAPGKKRPVEHSPVEPDSSSGTGKAGQQPARKRLNFGQTGDAD | | |
| SVPDPQPLGQPPAAPSGLGTNTMATGSGAPMADNNEGADGVGNSSGNWHCDSTWMGDRVI | | |
| TTSTRTWALPTYNNHLYKQISSQSGASNDNHYFGYSTPWGYFDFNRFHCHFSPRDWQRLI | | |
| NNNWGFRPKRLNFKLFNIQVKEVTQNDGTTTIANNLTSTVQVFTDSEYQLPYVLGSAHQG | | |
| CLPPFPADVFMVPQYGYLTLNNGSQAVGRSSFYCLEYFPSQMLRTGNNFTFSYTFEDVPF | | |
| HSSYAHSQSLDRLMNPLIDQYLYYLSRTNTPSGTTTQSRLQFSQAGASDIRDQSRNWLPG | | |
| PCYRQQRVSKTSADNNNSEYSWTGATKYHLNGRDSLVNPGPAMASHKDDEEKFFPQSGVL | | |
| IFGKQGSEKTNVDIEKVMITDEEEIRTTNPVATEQYGSVSTNLORGNRQAATADVNTQGV | | |
| LPGMVWQDRDVYLQGPIWAKIPHTDGHFHPSPLMGGFGLKHPPPQILIKNTPVPANPSTT | | |
| FSAAKFASFITQYSTGQVSVEIEWELQKENSKRWNPEIQYTSNYNKSVNVDFTVDTNGVY | | |
| SEPRPIGTRYLTRNL | | |
| SEQ ID NO: 8 | | |
| 1 | LGETTRP | 7 |
| SEQ ID NO: 9 | | |
| 1 | FSYTFEDVPF HSSYAHSQSL DRLMNPLIDQ YLYYLSRTNT PSGTTTQSRL | 50 |
| 51 | QFSQAGASDI RDQSRNWLPG PCYRQQRVSK TSADNNNSEY SWTGATKYHL | 100 |
| 101 | NGRDSLVNPG PAMASHKDDE EKFFPQSGVL IFGKQGSEKT NVDIEKVMIT | 150 |
| 151 | DEEEIRTTNP VATEQYGSVS TNLQRGNLAL GETTRPARQA ATADVNTQGV | 200 |
| 201 | LPGMVWQDRD VYLQGPIWAK IPHTDGHFHP SPLMGGFGLK HPPPQILIKN | 250 |
| -human ″wild-type″ CNGB3 cDNA | | |
| SEQ ID NO: 10 | | |
| 1 | ATGTTTAAAT CGCTGACAAA AGTCAACAAG GTGAAGCCTA TAGGAGAGAA | |
| 51 | CAATGAGAAT GAACAAAGTT CTCGTCGGAA TGAAGAAGGC TCTCACCCAA | |
| 100 | GTAATCAGTC TCAGCAAACC ACAGCACAGG AAGAAAACAA AGGTGAAGAG | |
| 151 | AAATCTCTCA AAACCAAGTC AACTCCAGTC ACGTCTGAAG AGCCACACAC | |
| 201 | CAACATACAA GACAAACTCT CCAAGAAAAA TTCCTCTGGA GATCTGACCA | |
| 251 | CAAACCCTGA CCCTCAAAAT GCAGCAGAAC CAACTGGAAC AGTGCCAGAG | |
| 301 | CAGAAGGAAA TGGACCCCGG GAAAGAAGGT CCAAACAGCC CACAAAACAA | |
| 351 | ACCGCCTGCA GCTCCTGTTA TAAATGAGTA TGCCGATGCC CAGCTACACA | |
| 401 | ACCTGGTGAA AAGAATGCGT CAAAGAACAG CCCTCTACAA GAAAAAGTTG | |
| 451 | GTAGAGGGAG ATCTCTCCTC ACCCGAAGCC AGCCCACAAA CTGCAAAGCC | |
| 501 | CACGGCTGTA CCACCAGTAA AAGAAAGCGA TGATAAGCCA ACAGAACATT | |
| 551 | ACTACAGGCT GTTGTGGTTC AAAGTCAAAA AGATGCCTTT AACAGAGTAC | |
| 601 | TTAAAGCGAA TTAAACTTCC AAACAGCATA GATTCATACA CAGATCGACT | |
| 651 | CTATCTCCTG TGGCTCTTGC TTGTCACTCT TGCCTATAAC TGGAACTGCT | |
| 701 | GTTTTATACC ACTGCGCCTC GTCTTCCCAT ATCAAACCGC AGACAACATA | |
| 751 | CACTACTGGC TTATTGCGGA CATCATATGT GATATCATCT ACCTTTATGA | |
| 801 | TATGCTATTT ATCCAGCCCA GACTCCAGTT TGTAAGAGGA GGAGACATAA | |
| 851 | TAGTGGATTC AAATGAGCTA AGGAAACACT ACAGGACTTC TACAAAATTT | |
| 901 | CAGTTGGATG TCGCATCAAT AATACCATTT GATATTTGCT ACCTCTTCTT | |
| 951 | TGGGTTTAAT CCAATGTTTA GAGCAAATAG GATGTTAAAG TACACTTCAT | |
| 1001 | TTTTTGAATT TAATCATCAC CTAGAGTCTA TAATGGACAA AGCATATATC | |
| 1051 | TACAGAGTTA TTCGAACAAC TGGATACTTG CTGTTTATTC TGCACATTAA | |
| 1101 | TGCCTGTGTT TATTACTGGG CTTCAAACTA TGAAGGAATT GGCACTACTA | |
| 1151 | GATGGGTGTA TGATGGGGAA GGAAACGAGT ATCTGAGATG TTATTATTGG | |
| 1201 | GCAGTTCGAA CTTTAATTAC CATTGGTGGC CTTCCAGAAC CACAAACTTT | |
| 1251 | ATTTGAAATT GTTTTTCAAC TCTTGAATTT TTTTTCTGGA GTTTTTGTGT | |
| 1301 | TCTCCAGTTT AATTGGTCAG ATGAGAGATG TGATTGGAGC AGCTACAGCC | |
| 1351 | AATCAGAACT ACTTCCGCGC CTGCATGGAT GACACCATTG CCTACATGAA | |
| 1401 | CAATTACTCC ATTCCTAAAC TTGTGCAAAA GCGAGTTCGG ACTTGGTATG | |
| 1451 | AATATACATG GGACTCTCAA AGAATGCTAG ATGAGTCTGA TTTGCTTAAG | |
| 1501 | ACCCTACCAA CTACGGTCCA GTTAGCCCTC GCCATTGATG TGAACTTCAG | |
| 1551 | CATCATCAGC AAAGTCGACT TGTTCAAGGG TTGTGATACA CAGATGATTT | |
| 1601 | ATGACATGTT GCTAAGATTG AAATCCGTTC TCTATTTGCC TGGTGACTTT | |
| 1651 | GTCTGCAAAA AGGGAGAAAT TGGCAAGGAA ATGTATATCA TCAAGCATGG | |
| 1701 | AGAAGTCCAA GTTCTTGGAG GCCCTGATGG TACTAAAGTT CTGGTTACTC | |
| 1751 | TGAAAGCTGG GTCGGTGTTT GGAGAAATCA GCCTTCTAGC AGCAGGAGGA | |
| 1801 | GGAAACCGTC GAACTGCCAA TGTGGTGGCC CACGGGTTTG CCAATCTTTT | |
| 1851 | AACTCTAGAC AAAAAGACCC TCCAAGAAAT TCTAGTGCAT TATCCAGATT | |
| 1901 | CTGAAAGGAT CCTCATGAAG AAAGCCAGAG TGCTTTTAAA GCAGAAGGCT | |
| 1951 | AAGACCGCAG AAGCAACCCC TCCAAGAAAA GATCTTGCCC TCCTCTTCCC | |
| 2001 | ACCGAAAGAA GAGACACCCA AACTGTTTAA AACTCTCCTA GGAGGCACAG | |
| 2051 | GAAAAGCAAG TCTTGCAAGA CTACTCAAAT TGAAGCGAGA GCAAGCAGCT | |
| 2101 | CAGAAGAAAG AAAATTCTGA AGGAGGAGAG GAAGAAGGAA AAGAAAATGA | |
| 2151 | AGATAAACAA AAAGAAAATG AAGATAAACA AAAAGAAAAT GAAGATAAAG | |
| 2201 | GAAAAGAAAA TGAAGATAAA GATAAAGGAA GAGAGCCAGA AGAGAAGCCA | |
| 2251 | CTGGACAGAC CTGAATGTAC AGCAAGTCCT ATTGCAGTGG AGGAAGAACC | |
| 2301 | CCACTCAGTT AGAAGGACAG TTTTACCCAG AGGGACTTCT CGTCAATCAC | |
| 2351 | TCATTATCAG CATGGCTCCT TCTGCTGAGG GCGGAGAAGA GGTTCTTACT | |
| 2401 | ATTGAAGTCA AAGAAAAGGC TAAGCAATGA | 2430 |
| -human ″wild-type″ CNGB3 protein | | |
| SEQ ID NO: 11 | | |
| 1 | MFKSLTKVNK VKPIGENNEN EQSSRRNEEG SHPSNQSQQT TAQEENKGEE | |
| 51 | KSLKTKSTPV TSEEPHTNIQ DKLSKKNSSG DLTTNPDPQN AAEPTGTVPE | |
| 100 | QKEMDPGKEG PNSPQNKPPA APVINEYADA QLHNLVKRMR QRTALYKKKL | |
| 151 | VEGDLSSPEA SPQTAKPTAV PPVKESDDKP TEHYYRLLWF KVKKMPLTEY | |
| 201 | LKRIKLPNSI DSYTDRLYLL WLLLVTLAYN WNCCFIPLRL VFPYQTADNI | |
| 251 | HYWLIADIIC DIIYLYDMLF IQPRLQFVRG GDIIVDSNEL RKHYRTSTKF | |
| 301 | QLDVASIIPF DICYLFFGFN PMFRANRMLK YTSFFEFNHH LESIMDKAYI | |
| 351 | YRVIRTTGYL LFILHINACV YYWASNYEGI GTTRWVYDGE GNEYLRCYYW | |
| 401 | AVRTLITIGG LPEPQTLFEI VFQLLNFFSG VFVFSSLIGQ MRDVIGAATA | |
| 451 | NQNYFRACMD DTIAYMNNYS IPKLVQKRVR TWYEYTWDSQ RMLDESDLLK | |
| 501 | TLPTTVQLAL AIDVNFSIIS KVDLFKGCDT QMIYDMLLRL KSVLYLPGDF | |
| 551 | VCKKGEIGKE MYIIKHGEVQ VLGGPDGTKV LVTLKAGSVF GEISLLAAGG | |
| 601 | GNRRTANVVA HGFANLLTLD KKTLQEILVH YPDSERILMK KARVLLKQKA | |
| 651 | KTAEATPPRK DLALLFPPKE ETPKLFKTLL GGTGKASLAR LLKLKREQAA | |
| 701 | QKKENSEGGE EEGKENEDKQ KENEDKQKEN EDKGKENEDK DKGREPEEKP | |
| 751 | LDRPECTASP IAVEEEPHSV RRTVLPRGTS RQSLIISMAP SAEGGEEVLT | |
| 801 | IEVKEKAKQ | 809 |

-ITR AAV2
SEQ ID NO: 12
GCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGG
TTCCTTGTAGTTAATGATTAA
-LCR
SEQ ID NO: 13
CCTACAGCAGCCAGGGTGAGATTATGAGGCTGAGCTGAGAATATCAAGACTGTACCGAGTAGG
GGGCCTTGGCAAGTGTGGAGAGCCCGGCAGCTGGGGCAGAGGGCGGAGTACGGTGTGCGTT
TACGGACCTCTTCAAACGAGGTAGGAAGGTCAGAAGTCAAAAAGGGAACAAATGATGTTTAAC
CACACAAAAATGAAAATCCAATGGTTGGATATCCATTCCAAATACACAAAGGCAACGGATAAGT
GATCCGGGCCAGGCACAGAAGGCCATGCACCCGTAGGATTGCACTCAGAGCTCCCAAATGCA
TAGGAATAGAAGGGTGGGTGCAGGAGGCTGAGGGGTGGGGAAAGGGCATGGGTGTTTCATGA
GGACAGAGCTTCCGTTTCATGCAATGAAAAGAGTTTGGAGACGGATGGTGGTGACTGGACTATA
CACTTACACACGGTAGCGATGGTACACTTTGTATTATGTATATTTTACCACGATCTTTTTAAAGTG
TCAAAGGCAAATGGCCAAATGGTTCCTTGTCCTATAGCTGTAGCAGCCATCGGCTGTTAGTGAC
AAAGCCCCTGAGTCAAGATGACAGCAGCCCCCATAACTCCTAATCGGCTCTCCCGCGTGGAGT
CATTTAGGAGTAGTCGCATTAGAGACAAGTCCAACATCTAATCTTCCACCCTGGCCAGGGCCCC
AGCTGGCAGCGAGGGTGGGAGACTCCGGGCAGAGCAGAGGGCGCTGACATTGGGGCCCGGC
CTGGCTTGGGTCCCTCTGGCCTTTCCCCAGGGGCCCTCTTTCCTTGGGGCTTTCTTGGGCCGCC
ACTGCTCCCGCTCCTCTCCCCCCATCCCACCCCCTCACCCCCTCGTTCTTCATATCCTTCTCTAG
TGCTCCCTCCACTTTCATCCACCCTTCTGCAAGAGTGTGGGACCACAAATGAGTTTTCACCTGGC
CTGGGGACACACGTGCCCCCACAGGTGCTGAGTGACTTTCTAGGACAGTAATCTGCTTTAGGCT
AAAATGGGACTTGATCTTCTGTTAGCCCTAATCATCAATTAGCAGAGCCGGTGAAGGTGCAGAA
CCTACCGCCTTTCCAGGCCTCCTCCCACCTCTGCCACCTCCACTCTCCTTCCTGGGATGTGGGG
GCTGGCACACGTGTGGCCCAGGGCATTGGTGGGATTGCACTGAGCTGGGTCATTAGCGTAATC
CTGGACAAGGGCAGACAGGGCGAGCGGAGGGCCAGCTCCGGGGCTCAGGCAAGGCTGGGG
GCTTCCCCCAGACACCCCACTCCTCCTCTGCTGGACCCCCACTTCATAGGGCACTTCGTGTTCT
CAAAGGGCTTCCAAATAGCATGGTGGCCTTGGATGCCCAGGGAAGCCTCAGAGTTGCTTATCT
CCCTCTAGACAGAAGGGGAATCTCGGTCAAGAGGGAGAGGTCGCCCTGTTCAAGGCCACCCA
GCCAGCTCATGGCGGTAATGGGACAAGGCTGGCCAGCCATCCCACCCTCAGAAGGGACCCGG
TGGGGCAGGTGATCTCAGAGGAGGCTCACTTCTGGGTCTCACATTCTT
-PR1.7
SEQ ID NO: 14
GGAGGCTGAGGGGTGGGGAAAGGGCATGGGTGTTTCATGAGGACAGAGCTTCCGTTTCATGC
AATGAAAAGAGTTTGGAGACGGATGGTGGTGACTGGACTATACACTTACACACGGTAGCGATG
GTACACTTTGTATTATGTATATTTTACCACGATCTTTTTAAAGTGTCAAAGGCAAATGGCCAAATG
GTTCCTTGTCCTATAGCTGTAGCAGCCATCGGCTGTTAGTGACAAAGCCCCTGAGTCAAGATGA
CAGCAGCCCCCATAACTCCTAATCGGCTCTCCCGCGTGGAGTCATTTAGGAGTAGTCGCATTAG
AGACAAGTCCAACATCTAATCTTCCACCCTGGCCAGGGCCCCAGCTGGCAGCGAGGGTGGGA
GACTCCGGGCAGAGCAGAGGGCGCTGACATTGGGGCCCGGCCTGGCTTGGGTCCCTCTGGCC
TTTCCCCAGGGGCCCTCTTTCCTTGGGGCTTTCTTGGGCCGCCACTGCTCCCGCTCCTCTCCCC
CCATCCCACCCCCTCACCCCCTCGTTCTTCATATCCTTCTCTAGTGCTCCCTCCACTTTCATCCAC
CCTTCTGCAAGAGTGTGGGACCACAAATGAGTTTTCACCTGGCCTGGGGACACACGTGCCCCC
ACAGGTGCTGAGTGACTTTCTAGGACAGTAATCTGCTTTAGGCTAAAATGGGACTTGATCTTCTG
TTAGCCCTAATCATCAATTAGCAGAGCCGGTGAAGGTGCAGAACCTACCGCCTTTCCAGGCCTC
CTCCCACCTCTGCCACCTCCACTCTCCTTCCTGGGATGTGGGGGCTGGCACACGTGTGGCCCA
GGGCATTGGTGGGATTGCACTGAGCTGGGTCATTAGCGTAATCCTGGACAAGGGCAGACAGG
GCGAGCGGAGGGCCAGCTCCGGGGCTCAGGCAAGGCTGGGGGCTTCCCCCAGACACCCCAC
TCCTCCTCTGCTGGACCCCCACTTCATAGGGCACTTCGTGTTCTCAAAGGGCTTCCAAATAGCAT
GGTGGCCTTGGATGCCCAGGGAAGCCTCAGAGTTGCTTATCTCCCTCTAGACAGAAGGGGAAT
CTCGGTCAAGAGGGAGAGGTCGCCCTGTTCAAGGCCACCCAGCCAGCTCATGGCGGTAATGG
GACAAGGCTGGCCAGCCATCCCACCCTCAGAAGGGACCCGGTGGGGCAGGTGATCTCAGAG
GAGGCTCACTTCTGGGTCTCACATTCTTGGATCCGGTTCCAGGCCTCGGCCCTAAATAGTCTCC
CTGGGCTTTCAAGAGAACCACATGAGAAAGGAGGATTCGGGCTCTGAGCAGTTTCACCACCCA
CCCCCCAGTCTGCAAATCCTGACCCGTGGGTCCACCTGCCCCAAAGGCGGACGCAGGACAGT
AGAAGGGAACAGAGAACACATAAACACAGAGAGGGCCACAGCGGCTCCCACAGTCACCGCC
ACCTTCCTGGCGGGGATGGGTGGGGCGTCTGAGTTTGGTTCCCAGCAAATCCCTCTGAGCCGC
CCTTGCGGGCTCGCCTCAGGAGCAGGGGAGCAAGAGGTGGGAGGAGGAGGTCTAAGTCCCA
GGCCCAATTAAGAGATCAGGTAGTGTAGGGTTTGGGAGCTTTTAAGGTGAAGAGGCCCGGGCT
GATCCCACAGGCCAGTATAAAGCGCCGTGACCCTCAGGTGATGCGCCAGGGCCGGCTGCCGT
CGGGGACAGGGCTTTCCATAGCCAT
-M/L-opsin promoter
SEQ ID NO: 15
CCAGCAAATCCCTCTGAGCCGCCCCCGGGGGCTCGCCTCAGGAGCAAGGAAGCAAGGGGTG
GGAGGAGGAGGTCTAAGTCCCAGGCCCAATTAAGAGATCAGATGGTGTAGGATTTGGGAGCTT
TTAAGGTGAAGAGGCCCGGGCTGATCCCACTGGCCGGTATAAAGCACCGTGACCCTCAGGTG
ACGCACCAGGGCCGGCTGCCGTCGGGGACAGGGCTTTCCATAGCC
-Chimeric intron
SEQ ID NO: 16
CAGGTAAGTATCAAGGTTACAAGACAGGTTTAAGGAGACCAATAGAAACTGGGCTTGTCGAGA
CAGAGAAGACTCTTGCGTTTCTGATAGGCACCTATTGGTCTTACTGACATCCACTTTGCCTTTCTC
TCCACAGG
-5′UTR
SEQ ID NO: 17
CCCAGAGAGGAGACAGGCTAGC
-human beta catenin 1 5′UTR
SEQ ID NO: 18
CCGGTGGCGGCAGGATACAGCGGCTTCTGCGCGACTTATAAGAGCTCCTTGTGCGGCGCCATT
TTAAGCCTCTCGGTCTGTGGCAGCAGCGTTGGCCCG
-Optimized Kozak
SEQ ID NO: 19
GCCGCCACC
-Human Cngb3 cDNA codon optimized V11
SEQ ID NO: 20
ATGTTCAAGAGCCTCACCAAAGTCAACAAGGTCAAGCCAATTGGAGAGAACAATGAGAACGAA
CAGTCCAGCAGACGGAATGAGGAGGGATCCCACCCATCCAACCAGAGCCAGCAGACCACTGC
CCAAGAGGAAAACAAGGGAGAGGAAAAGTCCCTCAAGACCAAGTCCACCCCTGTCACCTCTG
AGGAACCCCACACCAACATCCAGGACAAGCTGTCCAAGAAGAACTCCTCAGGAGACTTGACCA
CCAACCCTGACCCCCAAAATGCAGCGGAGCCCACAGGCACTGTGCCGGAACAGAAGGAAATG
GACCCGGGAAAGGAGGGGCCTAACAGCCCTCAGAACAAGCCTCCAGCTGCCCCAGTGATCAA
CGAATATGCTGATGCCCAGCTTCACAACCTGGTCAAGCGCATGAGACAGAGGACTGCCCTGTA
CAAGAAGAAGCTTGTGGAAGGGGACCTGTCCAGCCCTGAGGCCTCCCCGCAAACTGCCAAGC
CCACGGCTGTGCCCCCTGTGAAAGAGTCGGATGACAAGCCCACTGAGCATTACTACCGCCTGC
TGTGGTTCAAAGTTAAGAAGATGCCCCTCACTGAATACCTGAAGCGCATCAAGCTACCTAACTC
CATTGACTCATACACTGACCGGCTCTACTTGCTGTGGCTGCTGCTTGTGACCCTTGCCTACAACT
GGAACTGCTGCTTCATCCCTCTGAGGCTGGTGTTCCCGTACCAAACTGCAGACAACATCCACTA
CTGGCTGATTGCTGACATCATCTGTGATATCATCTACCTCTATGACATGCTGTTCATCCAACCAA
GGCTGCAGTTCGTGAGAGGGGGAGACATCATTGTGGACTCCAATGAGCTCCGGAAGCACTACC
GCACCTCCACCAAGTTCCAGCTGGATGTGGCCTCCATCATCCCCTTTGACATCTGCTACCTGTTC
TTTGGATTCAACCCCATGTTCCGGGCCAACAGAATGCTGAAGTACACCTCCTTCTTTGAATTCAA
CCATCACCTGGAATCCATCATGGACAAGGCCTACATCTACCGGGTCATCCGCACCACTGGTTAC
CTGTTGTTCATCCTGCACATAAATGCCTGTGTCTACTATTGGGCCTCCAACTATGAAGGCATTGG
TACCACCAGATGGGTGTATGATGGAGAGGGCAATGAGTACCTCCGGTGCTACTACTGGGCAGT
GCGCACCCTGATCACAATTGGGGGCCTCCCTGAGCCCCAGACCCTGTTTGAAATTGTGTTCCAA
CTGCTGAACTTCTTCTCGGGAGTGTTTGTGTTCAGCAGCCTCATTGGCCAGATGAGAGATGTCAT
TGGAGCAGCCACTGCCAACCAGAACTACTTCAGGGCCTGCATGGATGACACCATTGCCTACAT
GAACAACTACTCCATTCCCAAGCTTGTGCAGAAGAGAGTGCGAACTTGGTATGAGTACACCTGG
GACTCCCAGAGGATGCTGGATGAGTCAGACTTACTCAAGACCCTGCCCACCACTGTGCAGCTT
GCCCTGGCCATTGATGTGAACTTCTCCATCATCTCCAAAGTGGACCTGTTCAAGGGCTGTGACA
CCCAGATGATCTACGACATGTTGCTGCGGCTGAAGTCGGTGCTCTACCTCCCTGGAGATTTTGT
GTGCAAGAAGGGAGAAATTGGGAAGGAAATGTACATCATCAAGCATGGAGAGGTCCAAGTGCT
GGGTGGCCCGGATGGCACCAAAGTGCTGGTCACCCTGAAGGCTGGCTCAGTGTTTGGAGAAAT
CAGCCTCTTGGCGGCTGGGGGGGGCAACAGGAGAACTGCCAATGTGGTAGCCCATGGCTTTG
CCAACCTCCTGACCCTTGACAAGAAAACCCTCCAGGAAATCCTGGTGCACTACCCGGACTCAG
AGAGAATCCTGATGAAGAAGGCCCGGGTGCTGCTGAAGCAGAAGGCCAAGACTGCAGAGGCC
ACCCCCCCACGCAAAGACCTGGCCCTCCTGTTCCCGCCCAAGGAAGAAACCCCAAAGCTGTTC
AAGACCCTCCTGGGTGGCACTGGGAAGGCCTCCCTGGCCCGCCTGTTGAAGCTCAAAAGGGA
ACAGGCAGCCCAGAAGAAGGAGAACTCAGAGGGGGGAGAGGAAGAGGGCAAAGAGAACGA
GGATAAGCAAAAGGAGAACGAAGATAAGCAGAAGGAAAACGAGGACAAGGGAAAAGAAAAT
GAGGACAAGGACAAGGGTCGGGAGCCTGAAGAGAAGCCCCTGGACCGGCCTGAATGCACTG
CCAGCCCCATTGCTGTGGAAGAAGAACCCCACAGTGTCAGAAGGACTGTGCTGCCGAGAGGC
ACCAGCCGGCAGTCCCTGATCATCAGCATGGCCCCTTCTGCGGAGGGTGGAGAAGAAGTGCT
GACCATTGAAGTCAAGGAAAAGGCCAAGCAGTAA
-eGFP cDNA
SEQ ID NO: 21
ATGGTGAGCAAGGGCGAGGAGCTGTTCACCGGGGTGGTGCCCATCCTGGTCGAGCTGGACGG
CGACGTAAACGGCCACAAGTTCAGCGTGTCCGGCGAGGGCGAGGGCGATGCCACCTACGGC
AAGCTGACCCTGAAGTTCATCTGCACCACCGGCAAGCTGCCCGTGCCCTGGCCCACCCTCGTG
ACCACCCTGACCTACGGCGTGCAGTGCTTCAGCCGCTACCCCGACCACATGAAGCAGCACGA
CTTCTTCAAGTCCGCCATGCCCGAAGGCTACGTCCAGGAGCGCACCATCTTCTTCAAGGACGA
CGGCAACTACAAGACCCGCGCCGAGGTGAAGTTCGAGGGCGACACCCTGGTGAACCGCATCG
AGCTGAAGGGCATCGACTTCAAGGAGGACGGCAACATCCTGGGGCACAAGCTGGAGTACAAC
TACAACAGCCACAACGTCTATATCATGGCCGACAAGCAGAAGAACGGCATCAAGGTGAACTTC
AAGATCCGCCACAACATCGAGGACGGCAGCGTGCAGCTCGCCGACCACTACCAGCAGAACAC
CCCCATCGGCGACGGCCCCGTGCTGCTGCCCGACAACCACTACCTGAGCACCCAGTCCGCCC
TGAGCAAAGACCCCAACGAGAAGCGCGATCACATGGTCCTGCTGGAGTTCGTGACCGCCGCC
GGGATCACTCTCGGCATGGACGAGCTGTACAAGTAA
-SV40 polyA
SEQ ID NO: 22
CAGACATGATAAGATACATTGATGAGTTTGGACAAACCACAACTAGAATGCAGTGAAAAAAATG
CTTTATTTGTGAAATTTGTGATGCTATTGCTTTATTTGTAACCATTATAAGCTGCAATAAACAAGTT
AACAACAACAATTGCATTCATTTTATGTTTCAGGTTCAGGGGGAGATGTGGGAGGTTTTTTAAAG
CAAGTAAAACCTCTACAAATGTGGTAAAATCGATAAGGATC
-SPA
SEQ ID NO: 23
AATAAAAGATCTTTATTTTCATTAGATCTGTGTGTTGGTTTTTTGTGTG
-Optimized lead sequence
SEQ ID NO: 24
TAGGATATCA
-Human beta tubulin gene (TUBB) 5′UTR sequence
SEQ ID NO: 25
TCCTGCCGTTGCGTTTGCACCTCGCTGCTCCAGCCTCTGGGGCGCATTCCAACCTTCCAGCCTG
CGACCTGCGGAGAAAAAAAATTACTTATTTTCTTGCCCCATACATACCTTGAGGCGAGCAAAAA
AATTAAATTTTAACC
-pADV960-MNTC-LS-CNGB3CoV11-SPA (FIG. 1F)
SEQ ID NO: 26
GCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGG
TTCCTTGTAGTTAATGATTAACACTAGTCCTACAGCAGCCAGGGTGAGATTATGAGGCTGAGCT
GAGAATATCAAGACTGTACCGAGTAGGGGGCCTTGGCAAGTGTGGAGAGCCCGGCAGCTGGG
GCAGAGGGCGGAGTACGGTGTGCGTTTACGGACCTCTTCAAACGAGGTAGGAAGGTCAGAAG
TCAAAAAGGGAACAAATGATGTTTAACCACACAAAAATGAAAATCCAATGGTTGGATATCCATTC
CAAATACACAAAGGCAACGGATAAGTGATCCGGGCCAGGCACAGAAGGCCATGCACCCGTAG
GATTGCACTCAGAGCTCCCAAATGCATAGGAATAGAAGGGTGGGTGCAGGAGGCTGAGGGGT
GGGGAAAGGGCATGGGTGTTTCATGAGGACAGAGCTTCCGTTTCATGCAATGAAAAGAGTTTG
GAGACGGATGGTGGTGACTGGACTATACACTTACACACGGTAGCGATGGTACACTTTGTATTAT
GTATATTTTACCACGATCTTTTTAAAGTGTCAAAGGCAAATGGCCAAATGGTTCCTTGTCCTATAG
CTGTAGCAGCCATCGGCTGTTAGTGACAAAGCCCCTGAGTCAAGATGACAGCAGCCCCCATAA
CTCCTAATCGGCTCTCCCGCGTGGAGTCATTTAGGAGTAGTCGCATTAGAGACAAGTCCAACAT
CTAATCTTCCACCCTGGCCAGGGCCCCAGCTGGCAGCGAGGGTGGGAGACTCCGGGCAGAGC
AGAGGGCGCTGACATTGGGGCCCGGCCTGGCTTGGGTCCCTCTGGCCTTTCCCCAGGGGCCC
TCTTTCCTTGGGGCTTTCTTGGGCCGCCACTGCTCCCGCTCCTCTCCCCCCATCCCACCCCCTC
ACCCCCTCGTTCTTCATATCCTTCTCTAGTGCTCCCTCCACTTTCATCCACCCTTCTGCAAGAGTG
TGGGACCACAAATGAGTTTTCACCTGGCCTGGGGACACACGTGCCCCCACAGGTGCTGAGTGA
CTTTCTAGGACAGTAATCTGCTTTAGGCTAAAATGGGACTTGATCTTCTGTTAGCCCTAATCATCA
ATTAGCAGAGCCGGTGAAGGTGCAGAACCTACCGCCTTTCCAGGCCTCCTCCCACCTCTGCCA
CCTCCACTCTCCTTCCTGGGATGTGGGGGCTGGCACACGTGTGGCCCAGGGCATTGGTGGGAT
TGCACTGAGCTGGGTCATTAGCGTAATCCTGGACAAGGGCAGACAGGGCGAGCGGAGGGCCA
GCTCCGGGGCTCAGGCAAGGCTGGGGGCTTCCCCCAGACACCCCACTCCTCCTCTGCTGGAC
CCCCACTTCATAGGGCACTTCGTGTTCTCAAAGGGCTTCCAAATAGCATGGTGGCCTTGGATGC
CCAGGGAAGCCTCAGAGTTGCTTATCTCCCTCTAGACAGAAGGGGAATCTCGGTCAAGAGGGA
GAGGTCGCCCTGTTCAAGGCCACCCAGCCAGCTCATGGCGGTAATGGGACAAGGCTGGCCAG
CCATCCCACCCTCAGAAGGGACCCGGTGGGGCAGGTGATCTCAGAGGAGGCTCACTTCTGGG
TCTCACATTCTTCCAGCAAATCCCTCTGAGCCGCCCCCGGGGGCTCGCCTCAGGAGCAAGGAA
GCAAGGGGTGGGAGGAGGAGGTCTAAGTCCCAGGCCCAATTAAGAGATCAGATGGTGTAGGA
TTTGGGAGCTTTTAAGGTGAAGAGGCCCGGGCTGATCCCACTGGCCGGTATAAAGCACCGTGA
CCCTCAGGTGACGCACCAGGGCCGGCTGCCGTCGGGGACAGGGCTTTCCATAGCCCAGGTAA
GTATCAAGGTTACAAGACAGGTTTAAGGAGACCAATAGAAACTGGGCTTGTCGAGACAGAGAA
GACTCTTGCGTTTCTGATAGGCACCTATTGGTCTTACTGACATCCACTTTGCCTTTCTCTCCACAG
TAGGATATCAGGAGCCACCATGTTCAAGAGCCTCACCAAAGTCAACAAGGTCAAGCCAATTGG
AGAGAACAATGAGAACGAACAGTCCAGCAGACGGAATGAGGAGGGATCCCACCCATCCAACC
AGAGCCAGCAGACCACTGCCCAAGAGGAAAACAAGGGAGAGGAAAAGTCCCTCAAGACCAA
GTCCACCCCTGTCACCTCTGAGGAACCCCACACCAACATCCAGGACAAGCTGTCCAAGAAGAA
CTCCTCAGGAGACTTGACCACCAACCCTGACCCCCAAAATGCAGCGGAGCCCACAGGCACTG
TGCCGGAACAGAAGGAAATGGACCCGGGAAAGGAGGGGCCTAACAGCCCTCAGAACAAGCC
TCCAGCTGCCCCAGTGATCAACGAATATGCTGATGCCCAGCTTCACAACCTGGTCAAGCGCAT
GAGACAGAGGACTGCCCTGTACAAGAAGAAGCTTGTGGAAGGGGACCTGTCCAGCCCTGAGG
CCTCCCCGCAAACTGCCAAGCCCACGGCTGTGCCCCCTGTGAAAGAGTCGGATGACAAGCCC
ACTGAGCATTACTACCGCCTGCTGTGGTTCAAAGTTAAGAAGATGCCCCTCACTGAATACCTGA
AGCGCATCAAGCTACCTAACTCCATTGACTCATACACTGACCGGCTCTACTTGCTGTGGCTGCT
GCTTGTGACCCTTGCCTACAACTGGAACTGCTGCTTCATCCCTCTGAGGCTGGTGTTCCCGTAC
CAAACTGCAGACAACATCCACTACTGGCTGATTGCTGACATCATCTGTGATATCATCTACCTCTA
TGACATGCTGTTCATCCAACCAAGGCTGCAGTTCGTGAGAGGGGGAGACATCATTGTGGACTC
CAATGAGCTCCGGAAGCACTACCGCACCTCCACCAAGTTCCAGCTGGATGTGGCCTCCATCAT
CCCCTTTGACATCTGCTACCTGTTCTTTGGATTCAACCCCATGTTCCGGGCCAACAGAATGCTGA
AGTACACCTCCTTCTTTGAATTCAACCATCACCTGGAATCCATCATGGACAAGGCCTACATCTAC
CGGGTCATCCGCACCACTGGTTACCTGTTGTTCATCCTGCACATAAATGCCTGTGTCTACTATTG
GGCCTCCAACTATGAAGGCATTGGTACCACCAGATGGGTGTATGATGGAGAGGGCAATGAGTA
CCTCCGGTGCTACTACTGGGCAGTGCGCACCCTGATCACAATTGGGGGCCTCCCTGAGCCCCA
GACCCTGTTTGAAATTGTGTTCCAACTGCTGAACTTCTTCTCGGGAGTGTTTGTGTTCAGCAGCC
TCATTGGCCAGATGAGAGATGTCATTGGAGCAGCCACTGCCAACCAGAACTACTTCAGGGCCT
GCATGGATGACACCATTGCCTACATGAACAACTACTCCATTCCCAAGCTTGTGCAGAAGAGAGT
GCGAACTTGGTATGAGTACACCTGGGACTCCCAGAGGATGCTGGATGAGTCAGACTTACTCAA
GACCCTGCCCACCACTGTGCAGCTTGCCCTGGCCATTGATGTGAACTTCTCCATCATCTCCAAA
GTGGACCTGTTCAAGGGCTGTGACACCCAGATGATCTACGACATGTTGCTGCGGCTGAAGTCG
GTGCTCTACCTCCCTGGAGATTTTGTGTGCAAGAAGGGAGAAATTGGGAAGGAAATGTACATCA
TCAAGCATGGAGAGGTCCAAGTGCTGGGTGGCCCGGATGGCACCAAAGTGCTGGTCACCCTG
AAGGCTGGCTCAGTGTTTGGAGAAATCAGCCTCTTGGCGGCTGGGGGGGGCAACAGGAGAAC
TGCCAATGTGGTAGCCCATGGCTTTGCCAACCTCCTGACCCTTGACAAGAAAACCCTCCAGGAA
ATCCTGGTGCACTACCCGGACTCAGAGAGAATCCTGATGAAGAAGGCCCGGGTGCTGCTGAAG
CAGAAGGCCAAGACTGCAGAGGCCACCCCCCCACGCAAAGACCTGGCCCTCCTGTTCCCGCC
CAAGGAAGAAACCCCAAAGCTGTTCAAGACCCTCCTGGGTGGCACTGGGAAGGCCTCCCTGG
CCCGCCTGTTGAAGCTCAAAAGGGAACAGGCAGCCCAGAAGAAGGAGAACTCAGAGGGGGG
AGAGGAAGAGGGCAAAGAGAACGAGGATAAGCAAAAGGAGAACGAAGATAAGCAGAAGGAA
AACGAGGACAAGGGAAAAGAAAATGAGGACAAGGACAAGGGTCGGGAGCCTGAAGAGAAGC
CCCTGGACCGGCCTGAATGCACTGCCAGCCCCATTGCTGTGGAAGAAGAACCCCACAGTGTCA
GAAGGACTGTGCTGCCGAGAGGCACCAGCCGGCAGTCCCTGATCATCAGCATGGCCCCTTCT
GCGGAGGGTGGAGAAGAAGTGCTGACCATTGAAGTCAAGGAAAAGGCCAAGCAGTAAGCGG
CCGCAATAAAAGATCTTTATTTTCATTAGATCTGTGTGTTGGTTTTTTGTGTGTACGTAGTTAATCA
TTAACTACAAGGAACCCCTAGTGATGGAGTTGGCCACTCCCTCTCTGCGCGCTCGCTCGCTCAC
TGAGGCCGGGCGACCAAAGGTCGCCCGACGCCCGGGCTTTGCCCGGGCGGCCTCAGTGAGC
GAGCGAGCGCGC

| Name | Start | End |
|---|---|---|
| ITR, inverse terminal repeat from AAV2 | 1 | 145 |
| LCR, human opsin locus control region | 152 | 1716 |
| OPN1LW promoter, human OPN1LW core promoter | 1717 | 1947 |
| Chimeric intron | 1951 | 2083 |
| LS, Leader Sequence | 2084 | 2093 |
| OK, optimized Kozak | 2094 | 2102 |
| hCNGB3 codon optimized V11(CNGB3coV11), | 2103 | 4532 |
| codon optimized cDNA encoding human cyclic nucleotide | | |
| gated channel subunit beta 3 | | |
| SPA, short synthetic polyadenylation sequence | 4541 | 4589 |
| ITR, inverse terminal repeat from AAV2 | 4596 | 4740 |

-pADV961-MNTC-CNGB3CoV11-SPA (FIG. 1G)
SEQ ID NO: 27
GCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGG
TTCCTTGTAGTTAATGATTAACACTAGTCCTACAGCAGCCAGGGTGAGATTATGAGGCTGAGCT
GAGAATATCAAGACTGTACCGAGTAGGGGGCCTTGGCAAGTGTGGAGAGCCCGGCAGCTGGG
GCAGAGGGCGGAGTACGGTGTGCGTTTACGGACCTCTTCAAACGAGGTAGGAAGGTCAGAAG
TCAAAAAGGGAACAAATGATGTTTAACCACACAAAAATGAAAATCCAATGGTTGGATATCCATTC
CAAATACACAAAGGCAACGGATAAGTGATCCGGGCCAGGCACAGAAGGCCATGCACCCGTAG
GATTGCACTCAGAGCTCCCAAATGCATAGGAATAGAAGGGTGGGTGCAGGAGGCTGAGGGGT
GGGGAAAGGGCATGGGTGTTTCATGAGGACAGAGCTTCCGTTTCATGCAATGAAAAGAGTTTG
GAGACGGATGGTGGTGACTGGACTATACACTTACACACGGTAGCGATGGTACACTTTGTATTAT
GTATATTTTACCACGATCTTTTTAAAGTGTCAAAGGCAAATGGCCAAATGGTTCCTTGTCCTATAG
CTGTAGCAGCCATCGGCTGTTAGTGACAAAGCCCCTGAGTCAAGATGACAGCAGCCCCCATAA
CTCCTAATCGGCTCTCCCGCGTGGAGTCATTTAGGAGTAGTCGCATTAGAGACAAGTCCAACAT
CTAATCTTCCACCCTGGCCAGGGCCCCAGCTGGCAGCGAGGGTGGGAGACTCCGGGCAGAGC
AGAGGGCGCTGACATTGGGGCCCGGCCTGGCTTGGGTCCCTCTGGCCTTTCCCCAGGGGCCC
TCTTTCCTTGGGGCTTTCTTGGGCCGCCACTGCTCCCGCTCCTCTCCCCCCATCCCACCCCCTC
ACCCCCTCGTTCTTCATATCCTTCTCTAGTGCTCCCTCCACTTTCATCCACCCTTCTGCAAGAGTG
TGGGACCACAAATGAGTTTTCACCTGGCCTGGGGACACACGTGCCCCCACAGGTGCTGAGTGA
CTTTCTAGGACAGTAATCTGCTTTAGGCTAAAATGGGACTTGATCTTCTGTTAGCCCTAATCATCA
ATTAGCAGAGCCGGTGAAGGTGCAGAACCTACCGCCTTTCCAGGCCTCCTCCCACCTCTGCCA
CCTCCACTCTCCTTCCTGGGATGTGGGGGCTGGCACACGTGTGGCCCAGGGCATTGGTGGGAT
TGCACTGAGCTGGGTCATTAGCGTAATCCTGGACAAGGGCAGACAGGGCGAGCGGAGGGCCA
GCTCCGGGGCTCAGGCAAGGCTGGGGGCTTCCCCCAGACACCCCACTCCTCCTCTGCTGGAC
CCCCACTTCATAGGGCACTTCGTGTTCTCAAAGGGCTTCCAAATAGCATGGTGGCCTTGGATGC
CCAGGGAAGCCTCAGAGTTGCTTATCTCCCTCTAGACAGAAGGGGAATCTCGGTCAAGAGGGA
GAGGTCGCCCTGTTCAAGGCCACCCAGCCAGCTCATGGCGGTAATGGGACAAGGCTGGCCAG
CCATCCCACCCTCAGAAGGGACCCGGTGGGGCAGGTGATCTCAGAGGAGGCTCACTTCTGGG
TCTCACATTCTTCCAGCAAATCCCTCTGAGCCGCCCCCGGGGGCTCGCCTCAGGAGCAAGGAA
GCAAGGGGTGGGAGGAGGAGGTCTAAGTCCCAGGCCCAATTAAGAGATCAGATGGTGTAGGA
TTTGGGAGCTTTTAAGGTGAAGAGGCCCGGGCTGATCCCACTGGCCGGTATAAAGCACCGTGA
CCCTCAGGTGACGCACCAGGGCCGGCTGCCGTCGGGGACAGGGCTTTCCATAGCCCAGGTAA
GTATCAAGGTTACAAGACAGGTTTAAGGAGACCAATAGAAACTGGGCTTGTCGAGACAGAGAA
GACTCTTGCGTTTCTGATAGGCACCTATTGGTCTTACTGACATCCACTTTGCCTTTCTCTCCACAG
GCCGCCACCATGTTCAAGAGCCTCACCAAAGTCAACAAGGTCAAGCCAATTGGAGAGAACAAT
GAGAACGAACAGTCCAGCAGACGGAATGAGGAGGGATCCCACCCATCCAACCAGAGCCAGC
AGACCACTGCCCAAGAGGAAAACAAGGGAGAGGAAAAGTCCCTCAAGACCAAGTCCACCCCT
GTCACCTCTGAGGAACCCCACACCAACATCCAGGACAAGCTGTCCAAGAAGAACTCCTCAGGA
GACTTGACCACCAACCCTGACCCCCAAAATGCAGCGGAGCCCACAGGCACTGTGCCGGAACA
GAAGGAAATGGACCCGGGAAAGGAGGGGCCTAACAGCCCTCAGAACAAGCCTCCAGCTGCC
CCAGTGATCAACGAATATGCTGATGCCCAGCTTCACAACCTGGTCAAGCGCATGAGACAGAGG
ACTGCCCTGTACAAGAAGAAGCTTGTGGAAGGGGACCTGTCCAGCCCTGAGGCCTCCCCGCA
AACTGCCAAGCCCACGGCTGTGCCCCCTGTGAAAGAGTCGGATGACAAGCCCACTGAGCATTA
CTACCGCCTGCTGTGGTTCAAAGTTAAGAAGATGCCCCTCACTGAATACCTGAAGCGCATCAAG
CTACCTAACTCCATTGACTCATACACTGACCGGCTCTACTTGCTGTGGCTGCTGCTTGTGACCCT
TGCCTACAACTGGAACTGCTGCTTCATCCCTCTGAGGCTGGTGTTCCCGTACCAAACTGCAGAC
AACATCCACTACTGGCTGATTGCTGACATCATCTGTGATATCATCTACCTCTATGACATGCTGTTC
ATCCAACCAAGGCTGCAGTTCGTGAGAGGGGGAGACATCATTGTGGACTCCAATGAGCTCCGG
AAGCACTACCGCACCTCCACCAAGTTCCAGCTGGATGTGGCCTCCATCATCCCCTTTGACATCT
GCTACCTGTTCTTTGGATTCAACCCCATGTTCCGGGCCAACAGAATGCTGAAGTACACCTCCTTC
TTTGAATTCAACCATCACCTGGAATCCATCATGGACAAGGCCTACATCTACCGGGTCATCCGCA
CCACTGGTTACCTGTTGTTCATCCTGCACATAAATGCCTGTGTCTACTATTGGGCCTCCAACTAT
GAAGGCATTGGTACCACCAGATGGGTGTATGATGGAGAGGGCAATGAGTACCTCCGGTGCTAC
TACTGGGCAGTGCGCACCCTGATCACAATTGGGGGCCTCCCTGAGCCCCAGACCCTGTTTGAA
ATTGTGTTCCAACTGCTGAACTTCTTCTCGGGAGTGTTTGTGTTCAGCAGCCTCATTGGCCAGAT
GAGAGATGTCATTGGAGCAGCCACTGCCAACCAGAACTACTTCAGGGCCTGCATGGATGACAC
CATTGCCTACATGAACAACTACTCCATTCCCAAGCTTGTGCAGAAGAGAGTGCGAACTTGGTAT
GAGTACACCTGGGACTCCCAGAGGATGCTGGATGAGTCAGACTTACTCAAGACCCTGCCCACC
ACTGTGCAGCTTGCCCTGGCCATTGATGTGAACTTCTCCATCATCTCCAAAGTGGACCTGTTCAA
GGGCTGTGACACCCAGATGATCTACGACATGTTGCTGCGGCTGAAGTCGGTGCTCTACCTCCCT
GGAGATTTTGTGTGCAAGAAGGGAGAAATTGGGAAGGAAATGTACATCATCAAGCATGGAGAG
GTCCAAGTGCTGGGTGGCCCGGATGGCACCAAAGTGCTGGTCACCCTGAAGGCTGGCTCAGT
GTTTGGAGAAATCAGCCTCTTGGCGGCTGGGGGGGGCAACAGGAGAACTGCCAATGTGGTAG
CCCATGGCTTTGCCAACCTCCTGACCCTTGACAAGAAAACCCTCCAGGAAATCCTGGTGCACTA
CCCGGACTCAGAGAGAATCCTGATGAAGAAGGCCCGGGTGCTGCTGAAGCAGAAGGCCAAGA
CTGCAGAGGCCACCCCCCCACGCAAAGACCTGGCCCTCCTGTTCCCGCCCAAGGAAGAAACC
CCAAAGCTGTTCAAGACCCTCCTGGGTGGCACTGGGAAGGCCTCCCTGGCCCGCCTGTTGAAG
CTCAAAAGGGAACAGGCAGCCCAGAAGAAGGAGAACTCAGAGGGGGGAGAGGAAGAGGGC
AAAGAGAACGAGGATAAGCAAAAGGAGAACGAAGATAAGCAGAAGGAAAACGAGGACAAGG
GAAAAGAAAATGAGGACAAGGACAAGGGTCGGGAGCCTGAAGAGAAGCCCCTGGACCGGCC
TGAATGCACTGCCAGCCCCATTGCTGTGGAAGAAGAACCCCACAGTGTCAGAAGGACTGTGCT
GCCGAGAGGCACCAGCCGGCAGTCCCTGATCATCAGCATGGCCCCTTCTGCGGAGGGTGGAG
AAGAAGTGCTGACCATTGAAGTCAAGGAAAAGGCCAAGCAGTAAGCGGCCGCAATAAAAGAT
CTTTATTTTCATTAGATCTGTGTGTTGGTTTTTTGTGTGTACGTAGTTAATCATTAACTACAAGGAA
CCCCTAGTGATGGAGTTGGCCACTCCCTCTCTGCGCGCTCGCTCGCTCACTGAGGCCGGGCGA
CCAAAGGTCGCCCGACGCCCGGGCTTTGCCCGGGCGGCCTCAGTGAGCGAGCGAGCGCGC

| Name | Start | End |
|---|---|---|
| ITR, inverse terminal repeat from AAV2 | 1 | 145 |
| LCR, human opsin locus control region | 152 | 1716 |
| OPN1LW promoter, human OPN1LW core promoter | 1717 | 1947 |
| Chimeric intron | 1951 | 2083 |
| OK, optimized Kozak | 2084 | 2092 |
| hCNGB3 codon optimized V11(CNGB3coV11), | 2093 | 4522 |
| codon optimized cDNA encoding human cyclic nucleotide | | |
| gated channel subunit beta 3 | | |
| SPA, short synthetic polyadenylation sequence | 4531 | 4579 |
| ITR, inverse terminal repeat from AAV2 | 4586 | 4730 |

-pADV962-MNTC-hTUBB 5′UTR- CNGB3CoV11-SPA (FIG. 1H)
SEQ ID NO: 28
GCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGG
TTCCTTGTAGTTAATGATTAACACTAGTCCTACAGCAGCCAGGGTGAGATTATGAGGCTGAGCT
GAGAATATCAAGACTGTACCGAGTAGGGGGCCTTGGCAAGTGTGGAGAGCCCGGCAGCTGGG
GCAGAGGGCGGAGTACGGTGTGCGTTTACGGACCTCTTCAAACGAGGTAGGAAGGTCAGAAG
TCAAAAAGGGAACAAATGATGTTTAACCACACAAAAATGAAAATCCAATGGTTGGATATCCATTC
CAAATACACAAAGGCAACGGATAAGTGATCCGGGCCAGGCACAGAAGGCCATGCACCCGTAG
GATTGCACTCAGAGCTCCCAAATGCATAGGAATAGAAGGGTGGGTGCAGGAGGCTGAGGGGT
GGGGAAAGGGCATGGGTGTTTCATGAGGACAGAGCTTCCGTTTCATGCAATGAAAAGAGTTTG
GAGACGGATGGTGGTGACTGGACTATACACTTACACACGGTAGCGATGGTACACTTTGTATTAT
GTATATTTTACCACGATCTTTTTAAAGTGTCAAAGGCAAATGGCCAAATGGTTCCTTGTCCTATAG
CTGTAGCAGCCATCGGCTGTTAGTGACAAAGCCCCTGAGTCAAGATGACAGCAGCCCCCATAA
CTCCTAATCGGCTCTCCCGCGTGGAGTCATTTAGGAGTAGTCGCATTAGAGACAAGTCCAACAT
CTAATCTTCCACCCTGGCCAGGGCCCCAGCTGGCAGCGAGGGTGGGAGACTCCGGGCAGAGC
AGAGGGCGCTGACATTGGGGCCCGGCCTGGCTTGGGTCCCTCTGGCCTTTCCCCAGGGGCCC
TCTTTCCTTGGGGCTTTCTTGGGCCGCCACTGCTCCCGCTCCTCTCCCCCCATCCCACCCCCTC
ACCCCCTCGTTCTTCATATCCTTCTCTAGTGCTCCCTCCACTTTCATCCACCCTTCTGCAAGAGTG
TGGGACCACAAATGAGTTTTCACCTGGCCTGGGGACACACGTGCCCCCACAGGTGCTGAGTGA
CTTTCTAGGACAGTAATCTGCTTTAGGCTAAAATGGGACTTGATCTTCTGTTAGCCCTAATCATCA
ATTAGCAGAGCCGGTGAAGGTGCAGAACCTACCGCCTTTCCAGGCCTCCTCCCACCTCTGCCA
CCTCCACTCTCCTTCCTGGGATGTGGGGGCTGGCACACGTGTGGCCCAGGGCATTGGTGGGAT
TGCACTGAGCTGGGTCATTAGCGTAATCCTGGACAAGGGCAGACAGGGCGAGCGGAGGGCCA
GCTCCGGGGCTCAGGCAAGGCTGGGGGCTTCCCCCAGACACCCCACTCCTCCTCTGCTGGAC
CCCCACTTCATAGGGCACTTCGTGTTCTCAAAGGGCTTCCAAATAGCATGGTGGCCTTGGATGC
CCAGGGAAGCCTCAGAGTTGCTTATCTCCCTCTAGACAGAAGGGGAATCTCGGTCAAGAGGGA
GAGGTCGCCCTGTTCAAGGCCACCCAGCCAGCTCATGGCGGTAATGGGACAAGGCTGGCCAG
CCATCCCACCCTCAGAAGGGACCCGGTGGGGCAGGTGATCTCAGAGGAGGCTCACTTCTGGG
TCTCACATTCTTCCAGCAAATCCCTCTGAGCCGCCCCCGGGGGCTCGCCTCAGGAGCAAGGAA
GCAAGGGGTGGGAGGAGGAGGTCTAAGTCCCAGGCCCAATTAAGAGATCAGATGGTGTAGGA
TTTGGGAGCTTTTAAGGTGAAGAGGCCCGGGCTGATCCCACTGGCCGGTATAAAGCACCGTGA
CCCTCAGGTGACGCACCAGGGCCGGCTGCCGTCGGGGACAGGGCTTTCCATAGCCCAGGTAA
GTATCAAGGTTACAAGACAGGTTTAAGGAGACCAATAGAAACTGGGCTTGTCGAGACAGAGAA
GACTCTTGCGTTTCTGATAGGCACCTATTGGTCTTACTGACATCCACTTTGCCTTTCTCTCCACAG
TTCCTGCCGTTGCGTTTGCACCTCGCTGCTCCAGCCTCTGGGGCGCATTCCAACCTTCCAGCCT
GCGACCTGCGGAGAAAAAAAATTACTTATTTTCTTGCCCCATACATACCTTGAGGCGAGCAAAA
AAATTAAATTTTAACCGCCGCCACCATGTTCAAGAGCCTCACCAAAGTCAACAAGGTCAAGCCA
ATTGGAGAGAACAATGAGAACGAACAGTCCAGCAGACGGAATGAGGAGGGATCCCACCCATC
CAACCAGAGCCAGCAGACCACTGCCCAAGAGGAAAACAAGGGAGAGGAAAAGTCCCTCAAG
ACCAAGTCCACCCCTGTCACCTCTGAGGAACCCCACACCAACATCCAGGACAAGCTGTCCAAG
AAGAACTCCTCAGGAGACTTGACCACCAACCCTGACCCCCAAAATGCAGCGGAGCCCACAGG
CACTGTGCCGGAACAGAAGGAAATGGACCCGGGAAAGGAGGGGCCTAACAGCCCTCAGAAC
AAGCCTCCAGCTGCCCCAGTGATCAACGAATATGCTGATGCCCAGCTTCACAACCTGGTCAAG
CGCATGAGACAGAGGACTGCCCTGTACAAGAAGAAGCTTGTGGAAGGGGACCTGTCCAGCCC
TGAGGCCTCCCCGCAAACTGCCAAGCCCACGGCTGTGCCCCCTGTGAAAGAGTCGGATGACA
AGCCCACTGAGCATTACTACCGCCTGCTGTGGTTCAAAGTTAAGAAGATGCCCCTCACTGAATA
CCTGAAGCGCATCAAGCTACCTAACTCCATTGACTCATACACTGACCGGCTCTACTTGCTGTGG
CTGCTGCTTGTGACCCTTGCCTACAACTGGAACTGCTGCTTCATCCCTCTGAGGCTGGTGTTCCC
GTACCAAACTGCAGACAACATCCACTACTGGCTGATTGCTGACATCATCTGTGATATCATCTACC
TCTATGACATGCTGTTCATCCAACCAAGGCTGCAGTTCGTGAGAGGGGGAGACATCATTGTGGA
CTCCAATGAGCTCCGGAAGCACTACCGCACCTCCACCAAGTTCCAGCTGGATGTGGCCTCCAT
CATCCCCTTTGACATCTGCTACCTGTTCTTTGGATTCAACCCCATGTTCCGGGCCAACAGAATGC
TGAAGTACACCTCCTTCTTTGAATTCAACCATCACCTGGAATCCATCATGGACAAGGCCTACATC
TACCGGGTCATCCGCACCACTGGTTACCTGTTGTTCATCCTGCACATAAATGCCTGTGTCTACTA
TTGGGCCTCCAACTATGAAGGCATTGGTACCACCAGATGGGTGTATGATGGAGAGGGCAATGA
GTACCTCCGGTGCTACTACTGGGCAGTGCGCACCCTGATCACAATTGGGGGCCTCCCTGAGCC
CCAGACCCTGTTTGAAATTGTGTTCCAACTGCTGAACTTCTTCTCGGGAGTGTTTGTGTTCAGCA
GCCTCATTGGCCAGATGAGAGATGTCATTGGAGCAGCCACTGCCAACCAGAACTACTTCAGGG
CCTGCATGGATGACACCATTGCCTACATGAACAACTACTCCATTCCCAAGCTTGTGCAGAAGAG
AGTGCGAACTTGGTATGAGTACACCTGGGACTCCCAGAGGATGCTGGATGAGTCAGACTTACT
CAAGACCCTGCCCACCACTGTGCAGCTTGCCCTGGCCATTGATGTGAACTTCTCCATCATCTCC
AAAGTGGACCTGTTCAAGGGCTGTGACACCCAGATGATCTACGACATGTTGCTGCGGCTGAAG
TCGGTGCTCTACCTCCCTGGAGATTTTGTGTGCAAGAAGGGAGAAATTGGGAAGGAAATGTACA
TCATCAAGCATGGAGAGGTCCAAGTGCTGGGTGGCCCGGATGGCACCAAAGTGCTGGTCACC
CTGAAGGCTGGCTCAGTGTTTGGAGAAATCAGCCTCTTGGCGGCTGGGGGGGGCAACAGGAG
AACTGCCAATGTGGTAGCCCATGGCTTTGCCAACCTCCTGACCCTTGACAAGAAAACCCTCCAG
GAAATCCTGGTGCACTACCCGGACTCAGAGAGAATCCTGATGAAGAAGGCCCGGGTGCTGCTG
AAGCAGAAGGCCAAGACTGCAGAGGCCACCCCCCCACGCAAAGACCTGGCCCTCCTGTTCCC
GCCCAAGGAAGAAACCCCAAAGCTGTTCAAGACCCTCCTGGGTGGCACTGGGAAGGCCTCCC
TGGCCCGCCTGTTGAAGCTCAAAAGGGAACAGGCAGCCCAGAAGAAGGAGAACTCAGAGGG
GGGAGAGGAAGAGGGCAAAGAGAACGAGGATAAGCAAAAGGAGAACGAAGATAAGCAGAAG
GAAAACGAGGACAAGGGAAAAGAAAATGAGGACAAGGACAAGGGTCGGGAGCCTGAAGAGA
AGCCCCTGGACCGGCCTGAATGCACTGCCAGCCCCATTGCTGTGGAAGAAGAACCCCACAGT
GTCAGAAGGACTGTGCTGCCGAGAGGCACCAGCCGGCAGTCCCTGATCATCAGCATGGCCCC
TTCTGCGGAGGGTGGAGAAGAAGTGCTGACCATTGAAGTCAAGGAAAAGGCCAAGCAGTAAG
CGGCCGCAATAAAAGATCTTTATTTTCATTAGATCTGTGTGTTGGTTTTTTGTGTGTACGTAGTTAA
TCATTAACTACAAGGAACCCCTAGTGATGGAGTTGGCCACTCCCTCTCTGCGCGCTCGCTCGCT
CACTGAGGCCGGGCGACCAAAGGTCGCCCGACGCCCGGGCTTTGCCCGGGCGGCCTCAGTG
AGCGAGCGAGCGCGC

| Name | Start | End |
|---|---|---|
| ITR, inverse terminal repeat from AAV2 | 1 | 145 |
| LCR, human opsin locus control region | 152 | 1716 |
| OPN1LW promoter, human OPN1LW core promoter | 1717 | 1947 |
| Chimeric intron | 1951 | 2083 |
| Human beta tubulin gene (TUBB) 5′UTR | 2084 | 2227 |
| OK, optimized Kozak | 2228 | 2236 |
| hCNGB3 codon optimized V11(CNGB3coV11), | 2237 | 4666 |
| codon optimized cDNA encoding human cyclic nucleotide | | |
| gated channel subunit beta 3 | | |
| SPA, short synthetic polyadenylation sequence | 4675 | 4723 |
| ITR, inverse terminal repeat from AAV2 | 4730 | 4874 |

1. A polynucleotide cassette for enhanced expression of a transgene in cone cells of a mammalian retina, comprising:
(a) a promoter region, wherein the promoter region is specific for retinal cone cells;
(b) an optimized 5′ untranslated terminal repeat (UTR) sequence to increase translation of CNGB3 protein;
and
(c) a polyadenylation site.

2. The polynucleotide cassette of claim 1, further comprising at least one recombinant adeno-associated virus serotype 2 (AAV2) inverted terminal repeat (ITR).

3. The polynucleotide cassette of claim 2, further comprising two AAV2 ITR wherein one ITR is 5′ to the promoter and one ITR is 3′ to the polyadenylation site.

4. The polynucleotide cassette of claim 1, wherein the promoter region comprises SEQ ID NO: 14 or SEQ ID NO: 15.

5. The polynucleotide cassette of claim 4, wherein the promoter further comprises a human opsin locus control region (LCR).

6. The polynucleotide cassette of claim 5, wherein the LCR comprises SEQ ID NO: 13.

7. (canceled)

8. The polynucleotide cassette of claim 1, further comprising a unique coding sequence optimized for high level expression and low CpG operatively linked to the promoter region, wherein the coding sequence encodes a CNGB3 gene.

9. The polynucleotide cassette of claim 8, wherein the unique coding sequence is SEQ ID NO:10 or SEQ ID NO: 20, or a sequence having at least 75%, at least 80%, at least 85%, at least 90%, at least 95%, or at least 97% identity thereto.

10. (canceled)

11. The polynucleotide cassette of claim 1, wherein the polyadenylation site is SEQ ID NO: 22 or SEQ ID NO: 23.

12. (canceled)

13. A polynucleotide cassette comprising in 5′ to 3′ orientation:
(a) a first ITR comprising SEQ ID NO: 12;
(b) a human ops in locus control region comprising SEQ ID NO: 13;
(c) a promoter selected from SEQ ID NOs: 14 or 15;
(d) a chimeric intron comprising SEQ ID NO: 16;
(e) a 5′ UTR selected from comprising SEQ ID NOs: 17, 18 or 25;
(f) a 10 nt optimized lead sequence comprising SEQ ID NO:24;
(g) an optimized Kozak sequence comprising SEQ ID NO: 19;
(h) a nucleotide sequence encoding a therapeutic protein;
(i) a polyA encoding nucleotide sequence selected from a sequence comprising SEQ ID NOs: 22 or 23; and
(j) optionally, a second ITR comprising SEQ ID NO: 12;
wherein there is at least one ITR.

14-15. (canceled)

16. The polynucleotide cassette of claim 1 comprising SEQ ID NO: 3.

17-21. (canceled)

22. A recombinant virus comprising:
(a) a variant capsid protein; and
(b) the polynucleotide cassette of claim 1.

23. The recombinant virus of claim 22, wherein the recombinant virus is a recombinant adeno associated virus (AAV).

24. The recombinant virus of claim 23, wherein the capsid protein is an AAV variant 7m8 capsid protein or is derived from the AAV variant 7m8 capsid protein.

25. A pharmaceutical composition comprising the recombinant virus of claim 22 and a pharmaceutically acceptable excipient.

26. A method of treating achromotopsia in a human subject in need thereof, the method comprising administering to the subject the recombinant adeno-associated virus (rAAV) at a dosage ranging from about 1×10⁹ to about 1×10¹⁴ vector genomes (vg)/eye, wherein the rAAV comprises a CNGB3 gene, and wherein the rAAV comprises an AAV2 capsid variant that transduces foveal cone photoreceptors.

27. The method of claim 26, wherein the administration is selected from intravitreal (IVT) injection, subretinal (SR) injection, intraocular injection, or suprachoroidal injection.

28-38. (canceled)

39. An isolated host cell transfected or transduced with the polynucleotide cassette of claim 1.


SEQUENCE LISTING (36 sequences)

SEQ ID NO: 1 - DNA, 5174 bp, synthetic construct
  source 1..5174: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggatcttcaatattggccattagccatattattcattggttatatagcataaatcaatattggctattggccattgcatacgttgtatctatatcataatatgtacatttatattggctcatgtccaatatgaccgccatgttggcattgattattgactagtcctacagcagccagggtgagattatgaggctgagctgagaatatcaagactgtaccgagtagggggccttggcaagtgtggagagcccggcagctggggcagagggcggagtacggtgtgcgtttacggacctcttcaaacgaggtaggaaggtcagaagtcaaaaagggaacaaatgatgtttaaccacacaaaaatgaaaatccaatggttggatatccattccaaatacacaaaggcaacggataagtgatccgggccaggcacagaaggccatgcacccgtaggattgcactcagagctcccaaatgcataggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttccagcaaatccctctgagccgcccccgggggctcgcctcaggagcaaggaagcaaggggtgggaggaggaggtctaagtcccaggcccaattaagagatcagatggtgtaggatttgggagcttttaaggtgaagaggcccgggctgatcccactggccggtataaagcaccgtgaccctcaggtgacgcaccagggccggctgccgtcggggacagggctttccatagcccaggtaagtatcaaggttacaagacaggtttaaggagaccaatagaaactgggcttgtcgagacagagaagactcttgcgtttctgataggcacctattggtcttactgacatccactttgcctttctctccacaggcccagagaggagacaggctagcgccgccaccatgtttaaatcgctgacaaaagtcaacaaggtgaagcctataggagagaacaatgagaatgaacaaagttctcgtcggaatgaagaaggctctcacccaagtaatcagtctcagcaaaccacagcacaggaagaaaacaaaggtgaagagaaatctctcaaaaccaagtcaactccagtcacgtctgaagagccacacaccaacatacaagacaaactctccaagaaaaattcctctggagatctgaccacaaaccctgaccctcaaaatgcagcagaaccaactggaacagtgccagagcagaaggaaatggaccccgggaaagaaggtccaaacagcccacaaaacaaaccgcctgcagctcctgttataaatgagtatgccgatgcccagctacacaacctggtgaaaagaatgcgtcaaagaacagccctctacaagaaaaagttggtagagggagatctctcctcacccgaagccagcccacaaactgcaaagcccacggctgtaccaccagtaaaagaaagcgatgataagccaacagaacattactacaggctgttgtggttcaaagtcaaaaagatgcctttaacagagtacttaaagcgaattaaacttccaaacagcatagattcatacacagatcgactctatctcctgtggctcttgcttgtcactcttgcctataactggaactgctgttttataccactgcgcctcgtcttcccatatcaaaccgcagacaacatacactactggcttattgcggacatcatatgtgatatcatctacctttatgatatgctatttatccagcccagactccagtttgtaagaggaggagacataatagtggattcaaatgagctaaggaaacactacaggacttctacaaaatttcagttggatgtcgcatcaataataccatttgatatttgctacctcttctttgggtttaatccaatgtttagagcaaataggatgttaaagtacacttcattttttgaatttaatcatcacctagagtctataatggacaaagcatatatctacagagttattcgaacaactggatacttgctgtttattctgcacattaatgcctgtgtttattactgggcttcaaactatgaaggaattggcactactagatgggtgtatgatggggaaggaaacgagtatctgagatgttattattgggcagttcgaactttaattaccattggtggccttccagaaccacaaactttatttgaaattgtttttcaactcttgaattttttttctggagtttttgtgttctccagtttaattggtcagatgagagatgtgattggagcagctacagccaatcagaactacttccgcgcctgcatggatgacaccattgcctacatgaacaattactccattcctaaacttgtgcaaaagcgagttcggacttggtatgaatatacatgggactctcaaagaatgctagatgagtctgatttgcttaagaccctaccaactacggtccagttagccctcgccattgatgtgaacttcagcatcatcagcaaagtcgacttgttcaagggttgtgatacacagatgatttatgacatgttgctaagattgaaatccgttctctatttgcctggtgactttgtctgcaaaaagggagaaattggcaaggaaatgtatatcatcaagcatggagaagtccaagttcttggaggccctgatggtactaaagttctggttactctgaaagctgggtcggtgtttggagaaatcagccttctagcagcaggaggaggaaaccgtcgaactgccaatgtggtggcccacgggtttgccaatcttttaactctagacaaaaagaccctccaagaaattctagtgcattatccagattctgaaaggatcctcatgaagaaagccagagtgcttttaaagcagaaggctaagaccgcagaagcaacccctccaagaaaagatcttgccctcctcttcccaccgaaagaagagacacccaaactgtttaaaactctcctaggaggcacaggaaaagcaagtcttgcaagactactcaaattgaagcgagagcaagcagctcagaagaaagaaaattctgaaggaggagaggaagaaggaaaagaaaatgaagataaacaaaaagaaaatgaagataaacaaaaagaaaatgaagataaaggaaaagaaaatgaagataaagataaaggaagagagccagaagagaagccactggacagacctgaatgtacagcaagtcctattgcagtggaggaagaaccccactcagttagaaggacagttttacccagagggacttctcgtcaatcactcattatcagcatggctccttctgctgagggcggagaagaggttcttactattgaagtcaaagaaaaggctaagcaatgaatctagagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 2 - DNA, 4759 bp, synthetic construct
  source 1..4759: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacactagtcctacagcagccagggtgagattatgaggctgagctgagaatatcaagactgtaccgagtagggggccttggcaagtgtggagagcccggcagctggggcagagggcggagtacggtgtgcgtttacggacctcttcaaacgaggtaggaaggtcagaagtcaaaaagggaacaaatgatgtttaaccacacaaaaatgaaaatccaatggttggatatccattccaaatacacaaaggcaacggataagtgatccgggccaggcacagaaggccatgcacccgtaggattgcactcagagctcccaaatgcataggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttccagcaaatccctctgagccgcccccgggggctcgcctcaggagcaaggaagcaaggggtgggaggaggaggtctaagtcccaggcccaattaagagatcagatggtgtaggatttgggagcttttaaggtgaagaggcccgggctgatcccactggccggtataaagcaccgtgaccctcaggtgacgcaccagggccggctgccgtcggggacagggctttccatagcccaggtaagtatcaaggttacaagacaggtttaaggagaccaatagaaactgggcttgtcgagacagagaagactcttgcgtttctgataggcacctattggtcttactgacatccactttgcctttctctccacaggcccagagaggagacaggctagcgctagcgccgccaccatgttcaagagcctcaccaaagtcaacaaggtcaagccaattggagagaacaatgagaacgaacagtccagcagacggaatgaggagggatcccacccatccaaccagagccagcagaccactgcccaagaggaaaacaagggagaggaaaagtccctcaagaccaagtccacccctgtcacctctgaggaaccccacaccaacatccaggacaagctgtccaagaagaactcctcaggagacttgaccaccaaccctgacccccaaaatgcagcggagcccacaggcactgtgccggaacagaaggaaatggacccgggaaaggaggggcctaacagccctcagaacaagcctccagctgccccagtgatcaacgaatatgctgatgcccagcttcacaacctggtcaagcgcatgagacagaggactgccctgtacaagaagaagcttgtggaaggggacctgtccagccctgaggcctccccgcaaactgccaagcccacggctgtgccccctgtgaaagagtcggatgacaagcccactgagcattactaccgcctgctgtggttcaaagttaagaagatgcccctcactgaatacctgaagcgcatcaagctacctaactccattgactcatacactgaccggctctacttgctgtggctgctgcttgtgacccttgcctacaactggaactgctgcttcatccctctgaggctggtgttcccgtaccaaactgcagacaacatccactactggctgattgctgacatcatctgtgatatcatctacctctatgacatgctgttcatccaaccaaggctgcagttcgtgagagggggagacatcattgtggactccaatgagctccggaagcactaccgcacctccaccaagttccagctggatgtggcctccatcatcccctttgacatctgctacctgttctttggattcaaccccatgttccgggccaacagaatgctgaagtacacctccttctttgaattcaaccatcacctggaatccatcatggacaaggcctacatctaccgggtcatccgcaccactggttacctgttgttcatcctgcacataaatgcctgtgtctactattgggcctccaactatgaaggcattggtaccaccagatgggtgtatgatggagagggcaatgagtacctccggtgctactactgggcagtgcgcaccctgatcacaattgggggcctccctgagccccagaccctgtttgaaattgtgttccaactgctgaacttcttctcgggagtgtttgtgttcagcagcctcattggccagatgagagatgtcattggagcagccactgccaaccagaactacttcagggcctgcatggatgacaccattgcctacatgaacaactactccattcccaagcttgtgcagaagagagtgcgaacttggtatgagtacacctgggactcccagaggatgctggatgagtcagacttactcaagaccctgcccaccactgtgcagcttgccctggccattgatgtgaacttctccatcatctccaaagtggacctgttcaagggctgtgacacccagatgatctacgacatgttgctgcggctgaagtcggtgctctacctccctggagattttgtgtgcaagaagggagaaattgggaaggaaatgtacatcatcaagcatggagaggtccaagtgctgggtggcccggatggcaccaaagtgctggtcaccctgaaggctggctcagtgtttggagaaatcagcctcttggcggctggggggggcaacaggagaactgccaatgtggtagcccatggctttgccaacctcctgacccttgacaagaaaaccctccaggaaatcctggtgcactacccggactcagagagaatcctgatgaagaaggcccgggtgctgctgaagcagaaggccaagactgcagaggccacccccccacgcaaagacctggccctcctgttcccgcccaaggaagaaaccccaaagctgttcaagaccctcctgggtggcactgggaaggcctccctggcccgcctgttgaagctcaaaagggaacaggcagcccagaagaaggagaactcagaggggggagaggaagagggcaaagagaacgaggataagcaaaaggagaacgaagataagcagaaggaaaacgaggacaagggaaaagaaaatgaggacaaggacaagggtcgggagcctgaagagaagcccctggaccggcctgaatgcactgccagccccattgctgtggaagaagaaccccacagtgtcagaaggactgtgctgccgagaggcaccagccggcagtccctgatcatcagcatggccccttctgcggagggtggagaagaagtgctgaccattgaagtcaaggaaaaggccaagcagtaagcggccgcaataaaagatctttattttcattagatctgtgtgttggttttttgtgtgtacgtagttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 3 - DNA, 4830 bp, synthetic construct
  source 1..4830: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacactagtcctacagcagccagggtgagattatgaggctgagctgagaatatcaagactgtaccgagtagggggccttggcaagtgtggagagcccggcagctggggcagagggcggagtacggtgtgcgtttacggacctcttcaaacgaggtaggaaggtcagaagtcaaaaagggaacaaatgatgtttaaccacacaaaaatgaaaatccaatggttggatatccattccaaatacacaaaggcaacggataagtgatccgggccaggcacagaaggccatgcacccgtaggattgcactcagagctcccaaatgcataggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttccagcaaatccctctgagccgcccccgggggctcgcctcaggagcaaggaagcaaggggtgggaggaggaggtctaagtcccaggcccaattaagagatcagatggtgtaggatttgggagcttttaaggtgaagaggcccgggctgatcccactggccggtataaagcaccgtgaccctcaggtgacgcaccagggccggctgccgtcggggacagggctttccatagcccaggtaagtatcaaggttacaagacaggtttaaggagaccaatagaaactgggcttgtcgagacagagaagactcttgcgtttctgataggcacctattggtcttactgacatccactttgcctttctctccacaggccggtggcggcaggatacagcggcttctgcgcgacttataagagctccttgtgcggcgccattttaagcctctcggtctgtggcagcagcgttggcccggccgccaccatgttcaagagcctcaccaaagtcaacaaggtcaagccaattggagagaacaatgagaacgaacagtccagcagacggaatgaggagggatcccacccatccaaccagagccagcagaccactgcccaagaggaaaacaagggagaggaaaagtccctcaagaccaagtccacccctgtcacctctgaggaaccccacaccaacatccaggacaagctgtccaagaagaactcctcaggagacttgaccaccaaccctgacccccaaaatgcagcggagcccacaggcactgtgccggaacagaaggaaatggacccgggaaaggaggggcctaacagccctcagaacaagcctccagctgccccagtgatcaacgaatatgctgatgcccagcttcacaacctggtcaagcgcatgagacagaggactgccctgtacaagaagaagcttgtggaaggggacctgtccagccctgaggcctccccgcaaactgccaagcccacggctgtgccccctgtgaaagagtcggatgacaagcccactgagcattactaccgcctgctgtggttcaaagttaagaagatgcccctcactgaatacctgaagcgcatcaagctacctaactccattgactcatacactgaccggctctacttgctgtggctgctgcttgtgacccttgcctacaactggaactgctgcttcatccctctgaggctggtgttcccgtaccaaactgcagacaacatccactactggctgattgctgacatcatctgtgatatcatctacctctatgacatgctgttcatccaaccaaggctgcagttcgtgagagggggagacatcattgtggactccaatgagctccggaagcactaccgcacctccaccaagttccagctggatgtggcctccatcatcccctttgacatctgctacctgttctttggattcaaccccatgttccgggccaacagaatgctgaagtacacctccttctttgaattcaaccatcacctggaatccatcatggacaaggcctacatctaccgggtcatccgcaccactggttacctgttgttcatcctgcacataaatgcctgtgtctactattgggcctccaactatgaaggcattggtaccaccagatgggtgtatgatggagagggcaatgagtacctccggtgctactactgggcagtgcgcaccctgatcacaattgggggcctccctgagccccagaccctgtttgaaattgtgttccaactgctgaacttcttctcgggagtgtttgtgttcagcagcctcattggccagatgagagatgtcattggagcagccactgccaaccagaactacttcagggcctgcatggatgacaccattgcctacatgaacaactactccattcccaagcttgtgcagaagagagtgcgaacttggtatgagtacacctgggactcccagaggatgctggatgagtcagacttactcaagaccctgcccaccactgtgcagcttgccctggccattgatgtgaacttctccatcatctccaaagtggacctgttcaagggctgtgacacccagatgatctacgacatgttgctgcggctgaagtcggtgctctacctccctggagattttgtgtgcaagaagggagaaattgggaaggaaatgtacatcatcaagcatggagaggtccaagtgctgggtggcccggatggcaccaaagtgctggtcaccctgaaggctggctcagtgtttggagaaatcagcctcttggcggctggggggggcaacaggagaactgccaatgtggtagcccatggctttgccaacctcctgacccttgacaagaaaaccctccaggaaatcctggtgcactacccggactcagagagaatcctgatgaagaaggcccgggtgctgctgaagcagaaggccaagactgcagaggccacccccccacgcaaagacctggccctcctgttcccgcccaaggaagaaaccccaaagctgttcaagaccctcctgggtggcactgggaaggcctccctggcccgcctgttgaagctcaaaagggaacaggcagcccagaagaaggagaactcagaggggggagaggaagagggcaaagagaacgaggataagcaaaaggagaacgaagataagcagaaggaaaacgaggacaagggaaaagaaaatgaggacaaggacaagggtcgggagcctgaagagaagcccctggaccggcctgaatgcactgccagccccattgctgtggaagaagaaccccacagtgtcagaaggactgtgctgccgagaggcaccagccggcagtccctgatcatcagcatggccccttctgcggagggtggagaagaagtgctgaccattgaagtcaaggaaaaggccaagcagtaagcggccgcaataaaagatctttattttcattagatctgtgtgttggttttttgtgtgtacgtagttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 4 - DNA, 4725 bp, synthetic construct
  source 1..4725: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacactagtggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagccatggccaccatgttcaagagcctcaccaaagtcaacaaggtcaagccaattggagagaacaatgagaacgaacagtccagcagacggaatgaggagggatcccacccatccaaccagagccagcagaccactgcccaagaggaaaacaagggagaggaaaagtccctcaagaccaagtccacccctgtcacctctgaggaaccccacaccaacatccaggacaagctgtccaagaagaactcctcaggagacttgaccaccaaccctgacccccaaaatgcagcggagcccacaggcactgtgccggaacagaaggaaatggacccgggaaaggaggggcctaacagccctcagaacaagcctccagctgccccagtgatcaacgaatatgctgatgcccagcttcacaacctggtcaagcgcatgagacagaggactgccctgtacaagaagaagcttgtggaaggggacctgtccagccctgaggcctccccgcaaactgccaagcccacggctgtgccccctgtgaaagagtcggatgacaagcccactgagcattactaccgcctgctgtggttcaaagttaagaagatgcccctcactgaatacctgaagcgcatcaagctacctaactccattgactcatacactgaccggctctacttgctgtggctgctgcttgtgacccttgcctacaactggaactgctgcttcatccctctgaggctggtgttcccgtaccaaactgcagacaacatccactactggctgattgctgacatcatctgtgatatcatctacctctatgacatgctgttcatccaaccaaggctgcagttcgtgagagggggagacatcattgtggactccaatgagctccggaagcactaccgcacctccaccaagttccagctggatgtggcctccatcatcccctttgacatctgctacctgttctttggattcaaccccatgttccgggccaacagaatgctgaagtacacctccttctttgaattcaaccatcacctggaatccatcatggacaaggcctacatctaccgggtcatccgcaccactggttacctgttgttcatcctgcacataaatgcctgtgtctactattgggcctccaactatgaaggcattggtaccaccagatgggtgtatgatggagagggcaatgagtacctccggtgctactactgggcagtgcgcaccctgatcacaattgggggcctccctgagccccagaccctgtttgaaattgtgttccaactgctgaacttcttctcgggagtgtttgtgttcagcagcctcattggccagatgagagatgtcattggagcagccactgccaaccagaactacttcagggcctgcatggatgacaccattgcctacatgaacaactactccattcccaagcttgtgcagaagagagtgcgaacttggtatgagtacacctgggactcccagaggatgctggatgagtcagacttactcaagaccctgcccaccactgtgcagcttgccctggccattgatgtgaacttctccatcatctccaaagtggacctgttcaagggctgtgacacccagatgatctacgacatgttgctgcggctgaagtcggtgctctacctccctggagattttgtgtgcaagaagggagaaattgggaaggaaatgtacatcatcaagcatggagaggtccaagtgctgggtggcccggatggcaccaaagtgctggtcaccctgaaggctggctcagtgtttggagaaatcagcctcttggcggctggggggggcaacaggagaactgccaatgtggtagcccatggctttgccaacctcctgacccttgacaagaaaaccctccaggaaatcctggtgcactacccggactcagagagaatcctgatgaagaaggcccgggtgctgctgaagcagaaggccaagactgcagaggccacccccccacgcaaagacctggccctcctgttcccgcccaaggaagaaaccccaaagctgttcaagaccctcctgggtggcactgggaaggcctccctggcccgcctgttgaagctcaaaagggaacaggcagcccagaagaaggagaactcagaggggggagaggaagagggcaaagagaacgaggataagcaaaaggagaacgaagataagcagaaggaaaacgaggacaagggaaaagaaaatgaggacaaggacaagggtcgggagcctgaagagaagcccctggaccggcctgaatgcactgccagccccattgctgtggaagaagaaccccacagtgtcagaaggactgtgctgccgagaggcaccagccggcagtccctgatcatcagcatggccccttctgcggagggtggagaagaagtgctgaccattgaagtcaaggaaaaggccaagcagtaagcggccgcgcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcctacgtagttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 5 - DNA, 3237 bp, synthetic construct
  source 1..3237: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacactagtcctacagcagccagggtgagattatgaggctgagctgagaatatcaagactgtaccgagtagggggccttggcaagtgtggagagcccggcagctggggcagagggcggagtacggtgtgcgtttacggacctcttcaaacgaggtaggaaggtcagaagtcaaaaagggaacaaatgatgtttaaccacacaaaaatgaaaatccaatggttggatatccattccaaatacacaaaggcaacggataagtgatccgggccaggcacagaaggccatgcacccgtaggattgcactcagagctcccaaatgcataggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttccagcaaatccctctgagccgcccccgggggctcgcctcaggagcaaggaagcaaggggtgggaggaggaggtctaagtcccaggcccaattaagagatcagatggtgtaggatttgggagcttttaaggtgaagaggcccgggctgatcccactggccggtataaagcaccgtgaccctcaggtgacgcaccagggccggctgccgtcggggacagggctttccatagcccaggtaagtatcaaggttacaagacaggtttaaggagaccaatagaaactgggcttgtcgagacagagaagactcttgcgtttctgataggcacctattggtcttactgacatccactttgcctttctctccacaggcccagagaggagacaggctagcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatctacgtagttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 6 - protein, 10 aa, synthetic construct
LALGETTRPA

SEQ ID NO: 7 - protein, 735 aa, synthetic construct [sequence omitted]

SEQ ID NO: 8 - protein, 7 aa, synthetic construct
LGETTRP

SEQ ID NO: 9 - protein, 250 aa, synthetic construct
FSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTRPARQAATADVNTQGVLPGMVWQDRDVYLQGPIWAKIPHTDGHFHPSPLMGGFGLKHPPPQILIKN

SEQ ID NO: 10 - DNA, 2430 bp, synthetic construct
  source 1..2430: mol_type = other DNA
atgtttaaatcgctgacaaaagtcaacaaggtgaagcctataggagagaacaatgagaatgaacaaagttctcgtcggaatgaagaaggctctcacccaagtaatcagtctcagcaaaccacagcacaggaagaaaacaaaggtgaagagaaatctctcaaaaccaagtcaactccagtcacgtctgaagagccacacaccaacatacaagacaaactctccaagaaaaattcctctggagatctgaccacaaaccctgaccctcaaaatgcagcagaaccaactggaacagtgccagagcagaaggaaatggaccccgggaaagaaggtccaaacagcccacaaaacaaaccgcctgcagctcctgttataaatgagtatgccgatgcccagctacacaacctggtgaaaagaatgcgtcaaagaacagccctctacaagaaaaagttggtagagggagatctctcctcacccgaagccagcccacaaactgcaaagcccacggctgtaccaccagtaaaagaaagcgatgataagccaacagaacattactacaggctgttgtggttcaaagtcaaaaagatgcctttaacagagtacttaaagcgaattaaacttccaaacagcatagattcatacacagatcgactctatctcctgtggctcttgcttgtcactcttgcctataactggaactgctgttttataccactgcgcctcgtcttcccatatcaaaccgcagacaacatacactactggcttattgcggacatcatatgtgatatcatctacctttatgatatgctatttatccagcccagactccagtttgtaagaggaggagacataatagtggattcaaatgagctaaggaaacactacaggacttctacaaaatttcagttggatgtcgcatcaataataccatttgatatttgctacctcttctttgggtttaatccaatgtttagagcaaataggatgttaaagtacacttcattttttgaatttaatcatcacctagagtctataatggacaaagcatatatctacagagttattcgaacaactggatacttgctgtttattctgcacattaatgcctgtgtttattactgggcttcaaactatgaaggaattggcactactagatgggtgtatgatggggaaggaaacgagtatctgagatgttattattgggcagttcgaactttaattaccattggtggccttccagaaccacaaactttatttgaaattgtttttcaactcttgaattttttttctggagtttttgtgttctccagtttaattggtcagatgagagatgtgattggagcagctacagccaatcagaactacttccgcgcctgcatggatgacaccattgcctacatgaacaattactccattcctaaacttgtgcaaaagcgagttcggacttggtatgaatatacatgggactctcaaagaatgctagatgagtctgatttgcttaagaccctaccaactacggtccagttagccctcgccattgatgtgaacttcagcatcatcagcaaagtcgacttgttcaagggttgtgatacacagatgatttatgacatgttgctaagattgaaatccgttctctatttgcctggtgactttgtctgcaaaaagggagaaattggcaaggaaatgtatatcatcaagcatggagaagtccaagttcttggaggccctgatggtactaaagttctggttactctgaaagctgggtcggtgtttggagaaatcagccttctagcagcaggaggaggaaaccgtcgaactgccaatgtggtggcccacgggtttgccaatcttttaactctagacaaaaagaccctccaagaaattctagtgcattatccagattctgaaaggatcctcatgaagaaagccagagtgcttttaaagcagaaggctaagaccgcagaagcaacccctccaagaaaagatcttgccctcctcttcccaccgaaagaagagacacccaaactgtttaaaactctcctaggaggcacaggaaaagcaagtcttgcaagactactcaaattgaagcgagagcaagcagctcagaagaaagaaaattctgaaggaggagaggaagaaggaaaagaaaatgaagataaacaaaaagaaaatgaagataaacaaaaagaaaatgaagataaaggaaaagaaaatgaagataaagataaaggaagagagccagaagagaagccactggacagacctgaatgtacagcaagtcctattgcagtggaggaagaaccccactcagttagaaggacagttttacccagagggacttctcgtcaatcactcattatcagcatggctccttctgctgagggcggagaagaggttcttactattgaagtcaaagaaaaggctaagcaatga

SEQ ID NO: 11 - protein, 809 aa, synthetic construct
MFKSLTKVNKVKPIGENNENEQSSRRNEEGSHPSNQSQQTTAQEENKGEEKSLKTKSTPVTSEEPHTNIQDKLSKKNSSGDLTTNPDPQNAAEPTGTVPEQKEMDPGKEGPNSPQNKPPAAPVINEYADAQLHNLVKRMRQRTALYKKKLVEGDLSSPEASPQTAKPTAVPPVKESDDKPTEHYYRLLWFKVKKMPLTEYLKRIKLPNSIDSYTDRLYLLWLLLVTLAYNWNCCFIPLRLVFPYQTADNIHYWLIADIICDIIYLYDMLFIQPRLQFVRGGDIIVDSNELRKHYRTSTKFQLDVASIIPFDICYLFFGFNPMFRANRMLKYTSFFEFNHHLESIMDKAYIYRVIRTTGYLLFILHINACVYYWASNYEGIGTTRWVYDGEGNEYLRCYYWAVRTLITIGGLPEPQTLFEIVFQLLNFFSGVFVFSSLIGQMRDVIGAATANQNYFRACMDDTIAYMNNYSIPKLVQKRVRTWYEYTWDSQRMLDESDLLKTLPTTVQLALAIDVNFSIISKVDLFKGCDTQMIYDMLLRLKSVLYLPGDFVCKKGEIGKEMYIIKHGEVQVLGGPDGTKVLVTLKAGSVFGEISLLAAGGGNRRTANVVAHGFANLLTLDKKTLQEILVHYPDSERILMKKARVLLKQKAKTAEATPPRKDLALLFPPKEETPKLFKTLLGGTGKASLARLLKLKREQAAQKKENSEGGEEEGKENEDKQKENEDKQKENEDKGKENEDKDKGREPEEKPLDRPECTASPIAVEEEPHSVRRTVLPRGTSRQSLIISMAPSAEGGEEVLTIEVKEKAKQ

SEQ ID NO: 12 - DNA, 144 bp, synthetic construct
  source 1..144: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaa

SEQ ID NO: 13 - DNA, 1565 bp, synthetic construct
  source 1..1565: mol_type = other DNA
cctacagcagccagggtgagattatgaggctgagctgagaatatcaagactgtaccgagtagggggccttggcaagtgtggagagcccggcagctggggcagagggcggagtacggtgtgcgtttacggacctcttcaaacgaggtaggaaggtcagaagtcaaaaagggaacaaatgatgtttaaccacacaaaaatgaaaatccaatggttggatatccattccaaatacacaaaggcaacggataagtgatccgggccaggcacagaaggccatgcacccgtaggattgcactcagagctcccaaatgcataggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattctt

SEQ ID NO: 14 - DNA, 1726 bp, synthetic construct
  source 1..1726: mol_type = other DNA
ggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagccat

SEQ ID NO: 15 - DNA, 231 bp, synthetic construct
  source 1..231: mol_type = other DNA
ccagcaaatccctctgagccgcccccgggggctcgcctcaggagcaaggaagcaaggggtgggaggaggaggtctaagtcccaggcccaattaagagatcagatggtgtaggatttgggagcttttaaggtgaagaggcccgggctgatcccactggccggtataaagcaccgtgaccctcaggtgacgcaccagggccggctgccgtcggggacagggctttccatagcc

SEQ ID NO: 16 - DNA, 137 bp, synthetic construct
  source 1..137: mol_type = other DNA
caggtaagtatcaaggttacaagacaggtttaaggagaccaatagaaactgggcttgtcgagacagagaagactcttgcgtttctgataggcacctattggtcttactgacatccactttgcctttctctccacagg

SEQ ID NO: 17 - DNA, 22 bp, synthetic construct
  source 1..22: mol_type = other DNA
cccagagaggagacaggctagc

SEQ ID NO: 18 - DNA, 99 bp, synthetic construct
  source 1..99: mol_type = other DNA
ccggtggcggcaggatacagcggcttctgcgcgacttataagagctccttgtgcggcgccattttaagcctctcggtctgtggcagcagcgttggcccg

SEQ ID NO: 19 - skipped

SEQ ID NO: 20 - DNA, 2430 bp, synthetic construct
  source 1..2430: mol_type = other DNA
atgttcaagagcctcaccaaagtcaacaaggtcaagccaattggagagaacaatgagaacgaacagtccagcagacggaatgaggagggatcccacccatccaaccagagccagcagaccactgcccaagaggaaaacaagggagaggaaaagtccctcaagaccaagtccacccctgtcacctctgaggaaccccacaccaacatccaggacaagctgtccaagaagaactcctcaggagacttgaccaccaaccctgacccccaaaatgcagcggagcccacaggcactgtgccggaacagaaggaaatggacccgggaaaggaggggcctaacagccctcagaacaagcctccagctgccccagtgatcaacgaatatgctgatgcccagcttcacaacctggtcaagcgcatgagacagaggactgccctgtacaagaagaagcttgtggaaggggacctgtccagccctgaggcctccccgcaaactgccaagcccacggctgtgccccctgtgaaagagtcggatgacaagcccactgagcattactaccgcctgctgtggttcaaagttaagaagatgcccctcactgaatacctgaagcgcatcaagctacctaactccattgactcatacactgaccggctctacttgctgtggctgctgcttgtgacccttgcctacaactggaactgctgcttcatccctctgaggctggtgttcccgtaccaaactgcagacaacatccactactggctgattgctgacatcatctgtgatatcatctacctctatgacatgctgttcatccaaccaaggctgcagttcgtgagagggggagacatcattgtggactccaatgagctccggaagcactaccgcacctccaccaagttccagctggatgtggcctccatcatcccctttgacatctgctacctgttctttggattcaaccccatgttccgggccaacagaatgctgaagtacacctccttctttgaattcaaccatcacctggaatccatcatggacaaggcctacatctaccgggtcatccgcaccactggttacctgttgttcatcctgcacataaatgcctgtgtctactattgggcctccaactatgaaggcattggtaccaccagatgggtgtatgatggagagggcaatgagtacctccggtgctactactgggcagtgcgcaccctgatcacaattgggggcctccctgagccccagaccctgtttgaaattgtgttccaactgctgaacttcttctcgggagtgtttgtgttcagcagcctcattggccagatgagagatgtcattggagcagccactgccaaccagaactacttcagggcctgcatggatgacaccattgcctacatgaacaactactccattcccaagcttgtgcagaagagagtgcgaacttggtatgagtacacctgggactcccagaggatgctggatgagtcagacttactcaagaccctgcccaccactgtgcagcttgccctggccattgatgtgaacttctccatcatctccaaagtggacctgttcaagggctgtgacacccagatgatctacgacatgttgctgcggctgaagtcggtgctctacctccctggagattttgtgtgcaagaagggagaaattgggaaggaaatgtacatcatcaagcatggagaggtccaagtgctgggtggcccggatggcaccaaagtgctggtcaccctgaaggctggctcagtgtttggagaaatcagcctcttggcggctggggggggcaacaggagaactgccaatgtggtagcccatggctttgccaacctcctgacccttgacaagaaaaccctccaggaaatcctggtgcactacccggactcagagagaatcctgatgaagaaggcccgggtgctgctgaagcagaaggccaagactgcagaggccacccccccacgcaaagacctggccctcctgttcccgcccaaggaagaaaccccaaagctgttcaagaccctcctgggtggcactgggaaggcctccctggcccgcctgttgaagctcaaaagggaacaggcagcccagaagaaggagaactcagaggggggagaggaagagggcaaagagaacgaggataagcaaaaggagaacgaagataagcagaaggaaaacgaggacaagggaaaagaaaatgaggacaaggacaagggtcgggagcctgaagagaagcccctggaccggcctgaatgcactgccagccccattgctgtggaagaagaaccccacagtgtcagaaggactgtgctgccgagaggcaccagccggcagtccctgatcatcagcatggccccttctgcggagggtggagaagaagtgctgaccattgaagtcaaggaaaaggccaagcagtaa

SEQ ID NO: 21 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 22 - DNA, 237 bp, synthetic construct
  source 1..237: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatc

SEQ ID NO: 23 - DNA, 49 bp, synthetic construct
  source 1..49: mol_type = other DNA
aataaaagatctttattttcattagatctgtgtgttggttttttgtgtg

SEQ ID NO: 24 - DNA, 10 bp, synthetic construct
  source 1..10: mol_type = other DNA
taggatatca

SEQ ID NO: 25 - DNA, 143 bp, synthetic construct
  source 1..143: mol_type = other DNA
tcctgccgttgcgtttgcacctcgctgctccagcctctggggcgcattccaaccttccagcctgcgacctgcggagaaaaaaaattacttattttcttgccccatacataccttgaggcgagcaaaaaaattaaattttaacc

SEQ ID NO: 26 - DNA, 4740 bp, synthetic construct
  source 1..4740: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacactagtcctacagcagccagggtgagattatgaggctgagctgagaatatcaagactgtaccgagtagggggccttggcaagtgtggagagcccggcagctggggcagagggcggagtacggtgtgcgtttacggacctcttcaaacgaggtaggaaggtcagaagtcaaaaagggaacaaatgatgtttaaccacacaaaaatgaaaatccaatggttggatatccattccaaatacacaaaggcaacggataagtgatccgggccaggcacagaaggccatgcacccgtaggattgcactcagagctcccaaatgcataggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttccagcaaatccctctgagccgcccccgggggctcgcctcaggagcaaggaagcaaggggtgggaggaggaggtctaagtcccaggcccaattaagagatcagatggtgtaggatttgggagcttttaaggtgaagaggcccgggctgatcccactggccggtataaagcaccgtgaccctcaggtgacgcaccagggccggctgccgtcggggacagggctttccatagcccaggtaagtatcaaggttacaagacaggtttaaggagaccaatagaaactgggcttgtcgagacagagaagactcttgcgtttctgataggcacctattggtcttactgacatccactttgcctttctctccacagtaggatatcaggagccaccatgttcaagagcctcaccaaagtcaacaaggtcaagccaattggagagaacaatgagaacgaacagtccagcagacggaatgaggagggatcccacccatccaaccagagccagcagaccactgcccaagaggaaaacaagggagaggaaaagtccctcaagaccaagtccacccctgtcacctctgaggaaccccacaccaacatccaggacaagctgtccaagaagaactcctcaggagacttgaccaccaaccctgacccccaaaatgcagcggagcccacaggcactgtgccggaacagaaggaaatggacccgggaaaggaggggcctaacagccctcagaacaagcctccagctgccccagtgatcaacgaatatgctgatgcccagcttcacaacctggtcaagcgcatgagacagaggactgccctgtacaagaagaagcttgtggaaggggacctgtccagccctgaggcctccccgcaaactgccaagcccacggctgtgccccctgtgaaagagtcggatgacaagcccactgagcattactaccgcctgctgtggttcaaagttaagaagatgcccctcactgaatacctgaagcgcatcaagctacctaactccattgactcatacactgaccggctctacttgctgtggctgctgcttgtgacccttgcctacaactggaactgctgcttcatccctctgaggctggtgttcccgtaccaaactgcagacaacatccactactggctgattgctgacatcatctgtgatatcatctacctctatgacatgctgttcatccaaccaaggctgcagttcgtgagagggggagacatcattgtggactccaatgagctccggaagcactaccgcacctccaccaagttccagctggatgtggcctccatcatcccctttgacatctgctacctgttctttggattcaaccccatgttccgggccaacagaatgctgaagtacacctccttctttgaattcaaccatcacctggaatccatcatggacaaggcctacatctaccgggtcatccgcaccactggttacctgttgttcatcctgcacataaatgcctgtgtctactattgggcctccaactatgaaggcattggtaccaccagatgggtgtatgatggagagggcaatgagtacctccggtgctactactgggcagtgcgcaccctgatcacaattgggggcctccctgagccccagaccctgtttgaaattgtgttccaactgctgaacttcttctcgggagtgtttgtgttcagcagcctcattggccagatgagagatgtcattggagcagccactgccaaccagaactacttcagggcctgcatggatgacaccattgcctacatgaacaactactccattcccaagcttgtgcagaagagagtgcgaacttggtatgagtacacctgggactcccagaggatgctggatgagtcagacttactcaagaccctgcccaccactgtgcagcttgccctggccattgatgtgaacttctccatcatctccaaagtggacctgttcaagggctgtgacacccagatgatctacgacatgttgctgcggctgaagtcggtgctctacctccctggagattttgtgtgcaagaagggagaaattgggaaggaaatgtacatcatcaagcatggagaggtccaagtgctgggtggcccggatggcaccaaagtgctggtcaccctgaaggctggctcagtgtttggagaaatcagcctcttggcggctggggggggcaacaggagaactgccaatgtggtagcccatggctttgccaacctcctgacccttgacaagaaaaccctccaggaaatcctggtgcactacccggactcagagagaatcctgatgaagaaggcccgggtgctgctgaagcagaaggccaagactgcagaggccacccccccacgcaaagacctggccctcctgttcccgcccaaggaagaaaccccaaagctgttcaagaccctcctgggtggcactgggaaggcctccctggcccgcctgttgaagctcaaaagggaacaggcagcccagaagaaggagaactcagaggggggagaggaagagggcaaagagaacgaggataagcaaaaggagaacgaagataagcagaaggaaaacgaggacaagggaaaagaaaatgaggacaaggacaagggtcgggagcctgaagagaagcccctggaccggcctgaatgcactgccagccccattgctgtggaagaagaaccccacagtgtcagaaggactgtgctgccgagaggcaccagccggcagtccctgatcatcagcatggccccttctgcggagggtggagaagaagtgctgaccattgaagtcaaggaaaaggccaagcagtaagcggccgcaataaaagatctttattttcattagatctgtgtgttggttttttgtgtgtacgtagttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 27 - DNA, 4730 bp, synthetic construct
  source 1..4730: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacactagtcctacagcagccagggtgagattatgaggctgagctgagaatatcaagactgtaccgagtagggggccttggcaagtgtggagagcccggcagctggggcagagggcggagtacggtgtgcgtttacggacctcttcaaacgaggtaggaaggtcagaagtcaaaaagggaacaaatgatgtttaaccacacaaaaatgaaaatccaatggttggatatccattccaaatacacaaaggcaacggataagtgatccgggccaggcacagaaggccatgcacccgtaggattgcactcagagctcccaaatgcataggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttccagcaaatccctctgagccgcccccgggggctcgcctcaggagcaaggaagcaaggggtgggaggaggaggtctaagtcccaggcccaattaagagatcagatggtgtaggatttgggagcttttaaggtgaagaggcccgggctgatcccactggccggtataaagcaccgtgaccctcaggtgacgcaccagggccggctgccgtcggggacagggctttccatagcccaggtaagtatcaaggttacaagacaggtttaaggagaccaatagaaactgggcttgtcgagacagagaagactcttgcgtttctgataggcacctattggtcttactgacatccactttgcctttctctccacaggccgccaccatgttcaagagcctcaccaaagtcaacaaggtcaagccaattggagagaacaatgagaacgaacagtccagcagacggaatgaggagggatcccacccatccaaccagagccagcagaccactgcccaagaggaaaacaagggagaggaaaagtccctcaagaccaagtccacccctgtcacctctgaggaaccccacaccaacatccaggacaagctgtccaagaagaactcctcaggagacttgaccaccaaccctgacccccaaaatgcagcggagcccacaggcactgtgccggaacagaaggaaatggacccgggaaaggaggggcctaacagccctcagaacaagcctccagctgccccagtgatcaacgaatatgctgatgcccagcttcacaacctggtcaagcgcatgagacagaggactgccctgtacaagaagaagcttgtggaaggggacctgtccagccctgaggcctccccgcaaactgccaagcccacggctgtgccccctgtgaaagagtcggatgacaagcccactgagcattactaccgcctgctgtggttcaaagttaagaagatgcccctcactgaatacctgaagcgcatcaagctacctaactccattgactcatacactgaccggctctacttgctgtggctgctgcttgtgacccttgcctacaactggaactgctgcttcatccctctgaggctggtgttcccgtaccaaactgcagacaacatccactactggctgattgctgacatcatctgtgatatcatctacctctatgacatgctgttcatccaaccaaggctgcagttcgtgagagggggagacatcattgtggactccaatgagctccggaagcactaccgcacctccaccaagttccagctggatgtggcctccatcatcccctttgacatctgctacctgttctttggattcaaccccatgttccgggccaacagaatgctgaagtacacctccttctttgaattcaaccatcacctggaatccatcatggacaaggcctacatctaccgggtcatccgcaccactggttacctgttgttcatcctgcacataaatgcctgtgtctactattgggcctccaactatgaaggcattggtaccaccagatgggtgtatgatggagagggcaatgagtacctccggtgctactactgggcagtgcgcaccctgatcacaattgggggcctccctgagccccagaccctgtttgaaattgtgttccaactgctgaacttcttctcgggagtgtttgtgttcagcagcctcattggccagatgagagatgtcattggagcagccactgccaaccagaactacttcagggcctgcatggatgacaccattgcctacatgaacaactactccattcccaagcttgtgcagaagagagtgcgaacttggtatgagtacacctgggactcccagaggatgctggatgagtcagacttactcaagaccctgcccaccactgtgcagcttgccctggccattgatgtgaacttctccatcatctccaaagtggacctgttcaagggctgtgacacccagatgatctacgacatgttgctgcggctgaagtcggtgctctacctccctggagattttgtgtgcaagaagggagaaattgggaaggaaatgtacatcatcaagcatggagaggtccaagtgctgggtggcccggatggcaccaaagtgctggtcaccctgaaggctggctcagtgtttggagaaatcagcctcttggcggctggggggggcaacaggagaactgccaatgtggtagcccatggctttgccaacctcctgacccttgacaagaaaaccctccaggaaatcctggtgcactacccggactcagagagaatcctgatgaagaaggcccgggtgctgctgaagcagaaggccaagactgcagaggccacccccccacgcaaagacctggccctcctgttcccgcccaaggaagaaaccccaaagctgttcaagaccctcctgggtggcactgggaaggcctccctggcccgcctgttgaagctcaaaagggaacaggcagcccagaagaaggagaactcagaggggggagaggaagagggcaaagagaacgaggataagcaaaaggagaacgaagataagcagaaggaaaacgaggacaagggaaaagaaaatgaggacaaggacaagggtcgggagcctgaagagaagcccctggaccggcctgaatgcactgccagccccattgctgtggaagaagaaccccacagtgtcagaaggactgtgctgccgagaggcaccagccggcagtccctgatcatcagcatggccccttctgcggagggtggagaagaagtgctgaccattgaagtcaaggaaaaggccaagcagtaagcggccgcaataaaagatctttattttcattagatctgtgtgttggttttttgtgtgtacgtagttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 28 - DNA, 4874 bp, synthetic construct
  source 1..4874: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacactagtcctacagcagccagggtgagattatgaggctgagctgagaatatcaagactgtaccgagtagggggccttggcaagtgtggagagcccggcagctggggcagagggcggagtacggtgtgcgtttacggacctcttcaaacgaggtaggaaggtcagaagtcaaaaagggaacaaatgatgtttaaccacacaaaaatgaaaatccaatggttggatatccattccaaatacacaaaggcaacggataagtgatccgggccaggcacagaaggccatgcacccgtaggattgcactcagagctcccaaatgcataggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttccagcaaatccctctgagccgcccccgggggctcgcctcaggagcaaggaagcaaggggtgggaggaggaggtctaagtcccaggcccaattaagagatcagatggtgtaggatttgggagcttttaaggtgaagaggcccgggctgatcccactggccggtataaagcaccgtgaccctcaggtgacgcaccagggccggctgccgtcggggacagggctttccatagcccaggtaagtatcaaggttacaagacaggtttaaggagaccaatagaaactgggcttgtcgagacagagaagactcttgcgtttctgataggcacctattggtcttactgacatccactttgcctttctctccacagttcctgccgttgcgtttgcacctcgctgctccagcctctggggcgcattccaaccttccagcctgcgacctgcggagaaaaaaaattacttattttcttgccccatacataccttgaggcgagcaaaaaaattaaattttaaccgccgccaccatgttcaagagcctcaccaaagtcaacaaggtcaagccaattggagagaacaatgagaacgaacagtccagcagacggaatgaggagggatcccacccatccaaccagagccagcagaccactgcccaagaggaaaacaagggagaggaaaagtccctcaagaccaagtccacccctgtcacctctgaggaaccccacaccaacatccaggacaagctgtccaagaagaactcctcaggagacttgaccaccaaccctgacccccaaaatgcagcggagcccacaggcactgtgccggaacagaaggaaatggacccgggaaaggaggggcctaacagccctcagaacaagcctccagctgccccagtgatcaacgaatatgctgatgcccagcttcacaacctggtcaagcgcatgagacagaggactgccctgtacaagaagaagcttgtggaaggggacctgtccagccctgaggcctccccgcaaactgccaagcccacggctgtgccccctgtgaaagagtcggatgacaagcccactgagcattactaccgcctgctgtggttcaaagttaagaagatgcccctcactgaatacctgaagcgcatcaagctacctaactccattgactcatacactgaccggctctacttgctgtggctgctgcttgtgacccttgcctacaactggaactgctgcttcatccctctgaggctggtgttcccgtaccaaactgcagacaacatccactactggctgattgctgacatcatctgtgatatcatctacctctatgacatgctgttcatccaaccaaggctgcagttcgtgagagggggagacatcattgtggactccaatgagctccggaagcactaccgcacctccaccaagttccagctggatgtggcctccatcatcccctttgacatctgctacctgttctttggattcaaccccatgttccgggccaacagaatgctgaagtacacctccttctttgaattcaaccatcacctggaatccatcatggacaaggcctacatctaccgggtcatccgcaccactggttacctgttgttcatcctgcacataaatgcctgtgtctactattgggcctccaactatgaaggcattggtaccaccagatgggtgtatgatggagagggcaatgagtacctccggtgctactactgggcagtgcgcaccctgatcacaattgggggcctccctgagccccagaccctgtttgaaattgtgttccaactgctgaacttcttctcgggagtgtttgtgttcagcagcctcattggccagatgagagatgtcattggagcagccactgccaaccagaactacttcagggcctgcatggatgacaccattgcctacatgaacaactactccattcccaagcttgtgcagaagagagtgcgaacttggtatgagtacacctgggactcccagaggatgctggatgagtcagacttactcaagaccctgcccaccactgtgcagcttgccctggccattgatgtgaacttctccatcatctccaaagtggacctgttcaagggctgtgacacccagatgatctacgacatgttgctgcggctgaagtcggtgctctacctccctggagattttgtgtgcaagaagggagaaattgggaaggaaatgtacatcatcaagcatggagaggtccaagtgctgggtggcccggatggcaccaaagtgctggtcaccctgaaggctggctcagtgtttggagaaatcagcctcttggcggctggggggggcaacaggagaactgccaatgtggtagcccatggctttgccaacctcctgacccttgacaagaaaaccctccaggaaatcctggtgcactacccggactcagagagaatcctgatgaagaaggcccgggtgctgctgaagcagaaggccaagactgcagaggccacccccccacgcaaagacctggccctcctgttcccgcccaaggaagaaaccccaaagctgttcaagaccctcctgggtggcactgggaaggcctccctggcccgcctgttgaagctcaaaagggaacaggcagcccagaagaaggagaactcagaggggggagaggaagagggcaaagagaacgaggataagcaaaaggagaacgaagataagcagaaggaaaacgaggacaagggaaaagaaaatgaggacaaggacaagggtcgggagcctgaagagaagcccctggaccggcctgaatgcactgccagccccattgctgtggaagaagaaccccacagtgtcagaaggactgtgctgccgagaggcaccagccggcagtccctgatcatcagcatggccccttctgcggagggtggagaagaagtgctgaccattgaagtcaaggaaaaggccaagcagtaagcggccgcaataaaagatctttattttcattagatctgtgtgttggttttttgtgtgtacgtagttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 29 - protein, 7 aa, synthetic construct
ISDQTKA

SEQ ID NO: 30 - DNA, 20 bp, synthetic construct
  source 1..20: mol_type = other DNA
gaccctcaggtgacgcacca

SEQ ID NO: 31 - DNA, 20 bp, synthetic construct
  source 1..20: mol_type = other DNA
tcgacaagcccagtttctat

SEQ ID NO: 32 - DNA, 20 bp, synthetic construct
  source 1..20: mol_type = other DNA
cgtttctgataggcacctat

SEQ ID NO: 33 - DNA, 26 bp, synthetic construct
  source 1..26: mol_type = other DNA
gatcggaggaggaggtctaagtcccg

SEQ ID NO: 34 - DNA, 26 bp, synthetic construct
  source 1..26: mol_type = other DNA
gatcgggagcaggggagcaagaggtg

SEQ ID NO: 35 - DNA, 26 bp, synthetic construct
  source 1..26: mol_type = other DNA
gatcgcattaccgccatgagctggcg

SEQ ID NO: 36 - DNA, 26 bp, synthetic construct
  source 1..26: mol_type = other DNA
gatcgtataaagcaccgtgaccctcg